(19) United States
(12) Patent Application Publication
(10) Pub. No.: US 2023/0226206 A1
(43) Pub. Date: Jul. 20, 2023

(54) METHODS FOR THE USE OF A B7-H3 ANTIBODY-DRUG CONJUGATE ALONE OR IN COMBINATION

(71) Applicant: MACROGENICS, INC., Rockville, MD (US)

(72) Inventors: Gerry Chester BOHAC, Rockville, MD (US); Deryk LOO, Rockville, MD (US); Juniper A. SCRIBNER, Rockville, MD (US); Jon Marc WIGGINTON, Rockville, MD (US)

(21) Appl. No.: 17/998,455

(22) PCT Filed: May 10, 2021
(86) PCT No.: PCT/US2021/031598

§ 371 Date: Nov. 10, 2022

Related U.S. Application Data
(60) Provisional application No. 63/023,495, filed on May 12, 2020.
(60) Provisional application No. 63/180,795, filed on Apr. 28, 2021.

(51) Int. Cl. A61K 47/68; A61P 35/00; C07K 16/28; A61K 39/395; A61K 45/06; A61P 1/00

(52) U.S. Cl. CPC A61K 47/6803; A61P 35/00; A61K 47/6849; C07K 16/2827; A61K 47/6889; C07K 16/2818; A61K 39/3955; A61K 45/06; A61P 1/00; C07K 2317/24; C07K 2317/565; A61K 2039/507

(57) ABSTRACT

The present invention is directed to dosing regimens for administering a humanized anti-B7-H3 antibody conjugated to at least one duocarmycin moiety (a “B7-H3-ADC”) for the treatment of cancer, particularly a cancer associated with expression of B7-H3. The invention particularly concerns the use of such B7-H3-ADC optionally in combination with a PD-1 binding molecule for the treatment of cancer. The invention particularly concerns the use of such B7-H3-ADC and an anti-PD-1 antibody or a PD-1 X LAG-3 bispecific molecule. The invention is directed to the use of such molecules, and to the use of pharmaceutical compositions and pharmaceutical kits that contain such molecules and that facilitate the use of such dosing regimens in the treatment of cancer.

### CROSS-REFERENCE TO RELATED APPLCIATIONS

This application claims priority to U.S. Patent Application No. 63/023,495 (filed on May 12, 2020; pending), and U.S. Patent Application No. 63/180,795 (filed on Apr. 28, 2021; pending), each of which is incorporated herein by reference in its entirety for all purposes.

### REFERENCE TO SEQUENCE LISTING

This application includes a Sequence Listing pursuant to 37 C.F.R. 1.821 et seq., which has been submitted electronically in ASCII format and is hereby incorporated by reference in its entirety for all purposes. The ASCII copy of the Sequence Listing, created on May 5, 2021, is named MAC-0112-PC_SL.txt and is 49,512 bytes in size.

### FIELD OF THE INVENTION

The present invention is directed in part to dosing regimens for administering a humanized anti-B7-H3 antibody conjugated to a duocarmycin moiety (a “B7-H3-ADC”) for the treatment of cancer, particularly a cancer associated with expression of B7-H3. The invention in part concerns the use of such B7-H3-ADC optionally in combination with a PD-1 binding molecule for the treatment of cancer. The invention in part concerns the use of such B7-H3-ADC and an anti-PD-1 antibody, or a bispecific molecule capable of binding to PD-1 and LAG-3 (“PD-1 X LAG-3 bispecific molecule”). The invention is directed in part to the use of such molecules, and to the use of pharmaceutical compositions and pharmaceutical kits that contain such molecules and that facilitate the use of such dosing regimens in the treatment of cancer.

### BACKGROUND OF THE INVENTION

### I. B7 Superfamily and B7-H3

B7-H3 is a member of the B7-CD28 Superfamily and is expressed on antigen-presenting cells. B7-H3 is unique in that the major human form contains two extracellular tandem IgV-IgC domains (i.e., IgV-IgC-IgV-IgC) (Collins, M. et al. (2005) “The B7 Family Of Immune-Regulatory Ligands,” Genome Biol. 6:223.1-223.7). Although initially thought to comprise only 2 Ig domains (IgV-IgC, see, e.g., NCBI Sequence NP_079516) a four immunoglobulin extracellular domain variant (“4Ig-B7-H3”) has been identified and found to be the more common human form of the protein (Sharpe, A. H. et al. (2002) “The B7-CD28 Superfamily,” Nature Rev. Immunol. 2:116-126; also see, e.g., NCBI Sequence NP_001019907). B7-H3 mRNA expression has been found in heart, kidney, testes, lung, liver, pancreas, prostate, colon, and osteoblast cells (Collins, M. et al. (2005) “The B7 Family Of Immune-Regulatory Ligands,” Genome Biol. 6:223.1-223.7). At the protein level, B7-H3 is found in human liver, lung, bladder, testis, prostate, breast, placenta, and lymphoid organs (Hofmeyer, K. et al. (2008) “The Contrasting Role Of B7-H3,” Proc. Natl. Acad. Sci. (U.S.A.) 105(30):10277-10278).

Although B7-H3 is not expressed on resting B or T cells, monocytes, or dendritic cells, it is induced on dendritic cells by IFN-y and on monocytes by GM-CSF (Sharpe, A.H. et al. (2002) “The B7-CD28 Superfamily,” Nature Rev. Immunol. 2:116-126). The mode of action of B7-H3 is complex, and the protein is reported to mediate both T Cell co-stimulation and co-inhibition (Hofmeyer, K. et al. (2008) “The Contrasting Role Of B7-H3,” Proc. Natl. Acad. Sci. (U.S.A.) 105(30):10277-10278; Martin-Orozco, N. etal. (2007) “Inhibitory Costimulation And Anti-Tumor Immunity,” Semin. Cancer Biol. 17(4):288-298. B7-H3 binds to an unidentified receptor(s) to mediate co-inhibition of T cells. In addition, B7-H3, through interactions with unknown receptor(s) is an inhibitor for NK-cells and osteoblastic cells (Hofmeyer, K. etal. (2008) “The Contrasting Role Of B7-H3,” Proc. Natl. Acad. Sci. (U.S.A.) 105(30):10277-10278).

### II. B7-H3 Expressing Tumors

B7-H3 is expressed on a variety of cancer cells (e.g., neuroblastoma, gastric, ovarian, non-small cell lung cancers, etc., see, e.g., Modak, S., et al. (2001) “Monoclonal antibody 8H9 targets a novel cell surface antigen expressed by a wide spectrum of human solid tumors,” Cancer Res 61:4048-54) and cultured cancer stem-like cells. Several independent studies have shown that human malignant tumor cells exhibit a marked increase in expression of B7-H3 protein and that this increased expression was associated with increased disease severity (Tekle, C., et al. (2012) “B7-H3 Contributes To The Metastatic Capacity Of Melanoma Cells By Modulation Of Known Metastasis-Associated Genes,” Int. J. Cancer 130:2282-90; Wang, L., et al. (2013) “B7-H3 Mediated Tumor Immunology: Friend Or Foe?,” Int. J. Cancer 134(12):2764-2771), suggesting that B7-H3 is exploited by tumors as an immune evasion pathway (Hofmeyer, K. et al. (2008) “The Contrasting Role Of B7-H3,” Proc. Natl. Acad. Sci. (U.S.A.) 105(30):10277-10278).

The role of B7-H3 in inhibiting the immune system and the increased expression of B7-H3 on human tumors has suggested that this molecule might serve as a therapeutic target for the treatment of cancer. The use of anti-B7-H3 antibodies and other molecules that modulate B7-H3 expression to treat tumors and/or up-modulate an immune response has been proposed (see, Loo, D. et al. (2012) “Development of an Fc-Enhanced Anti-B7-H3 Monoclonal Antibody with Potent Antitumor Activity,” Clin Cancer Res; 18: 3834-3845; Ahmed, M. et al. (2015) “Humanized Affinity-Matured Monoclonal Antibody 8H9 Has Potent Anti-Tumor Activity and Binds to FG Loop of B7-H3,” J. Biol. Chem. 290: 30018-30029; Nagase-Zembutsu, A. et al. (2016) “Development of DS-5573a: A novel afucosylated monoclonal antibody directed at B7-H3 with potent antitumor activity,” Cancer Sci. 2016, doi: 10.1111/cas.12915;; see also, U.S. Pat. No. 7,279,567, 7,527,969,7,718,774, 8,779,098, 8,802,091, US Patent Publication Nos. 2002/0168762; 2008/0081346, 2008/0116219, 2013/0078234, 2015/0274838, PCT Publications Nos. WO 2009/073533; WO 2008/066691; WO 2006/016276; WO 2008/116219; WO 2001/094413, WO 2002/32375, WO 2004/093894, WO 2006/016276, WO 2008/116219, and WO 2011/109400.

### III. Cell-Mediated Immune Responses

The immune response is tightly controlled by co-stimulatory and co-inhibitory ligands and receptors often referred to as “immune checkpoints” (Chen et al., (2013) “Molecular Mechanisms of T Cell Co-Stimulation And Co-Inhibition,” Nature Rev. Immunol. 13:227-242; Pardoll, D. M., (2012) “The Blockade Of Immune Checkpoints In Cancer Immunotherapy,” Nat. Rev. Cancer 12(4):252-264). These molecules provide a balanced network of positive and negative signals that regulate immune responses to provide protection against infection and cancer. Some cancer cells are able to escape the immune system by engendering a state of T cell exhaustion in which T cells are exposed to persistent antigen and/or inflammatory signals (Wherry E. J. (2010) “T Cell Exhaustion,” Nat. Immunol. 12(6):492-499). Two immune checkpoint molecules involved in T cell exhaustion, Programmed Death-1 (“PD-1”) and Lymphocyte Activation Gene 3 (“LAG-3”) (Wherry, J. E. (2015) “Molecular And Cellular Insights Into T Cell Exhaustion,” Nat. Rev. Immunol. 15(8):486-499).

Programmed Death-1 (“PD-1,” also known as “CD279”) is an approximately 31 kD type I membrane protein member of the extended CD28/CTLA-4 family of T cell regulators that broadly negatively regulates immune responses (Ishida, Y. et al. (1992) “Induced Expression Of PD-1, A Novel Member Of The Immunoglobulin Gene Superfamily, Upon Programmed Cell Death,” EMBO J. 11:3887-3895. PD-1 mediates its inhibition of the immune system by binding to the transmembrane protein ligands: Programmed Death-Ligand 1 (“PD-L1,” also known as “B7-H1”) and Programmed Death-Ligand 2 (“PD-L2,” also known as “B7-DC”) (Flies, D.B. et al. (2007) “The New B7s: Playing a Pivotal Role in Tumor Immunity,” J. Immunother. 30(3):251-260).

The role of PD-1 ligand interactions in inhibiting T cell activation and proliferation has suggested that these biomolecules might serve as therapeutic targets for treatments of inflammation and cancer. The use of anti-PD-1 antibodies to treat tumors and up-modulate an adaptive immune response has been proposed and antibodies capable of specifically binding to PD-1 have been reported (see, e.g., Patnaik A. et al. (2015) “Phase I Study of Pembrolizumab (MK-3475; Anti—PD-1 Monoclonal Antibody) in Patients with Advanced Solid Tumors,” Clin Cancer Res; 21(19):4286-4293U.S. Pat. Nos. 7,488,802; ; 7,521,051; 7,595,048; 8,008,449; 8,354,509; 8,735,553; 8,779,105; 8,900,587; 9,084,776; 9,815,897 and 10,577,422; and PCT Patent Publication Nos. WO 2014/194302; and WO 2015/035606; WO 2004/056875; WO 2006/121168; WO 2008/156712; WO 2012/135408; WO 2012/145493; WO 2013/014668; WO 2014/179664; WO 2014/194302; WO 2015/112800; and W02019/246110).

Lymphocyte Activation Gene 3 (“LAG-3,” also known as “CD223”) is a cell-surface receptor protein that is expressed by activated CD4⁺ and CD8⁺ T cells and NK cells, and is constitutively expressed by plasmacytoid dendritic cells; LAG-3 is not expressed by B-cells, monocytes or any other cell types tested (Workman, C. J. et al. (2009) “LAG-3 Regulates Plasmacytoid Dendritic Cell Homeostasis,” J. Immunol. 182(4): 1885-1891).

Studies have shown that LAG-3 plays an important role in negatively regulating T cell proliferation, function and homeostasis and in T cell exhaustion (see, e.g., Workman, C. J. (2005) “Negative Regulation Of T-Cell Homeostasis By Lymphocyte Activation Gene-3 (CD223),” J. Immunol. 174:688-695) and have suggested that inhibiting LAG-3 function through antibody blockade can reverse LAG-3-mediated immune system inhibition and partially restore effector function (Grosso, J. F. et al. (2009) “Functionally Distinct LAG-3 and PD-1 Subsets on Activated and Chronically Stimulated CD8 T-Cells,” J. Immunol. 182(11):6659-6669;). Antibodies capable of specifically binding to LAG-3 have been reported (see, e.g., PCT Publication Nos. WO 2014/140180, WO 2015/138920, WO 2015/116539, WO 2016/028672, WO 2016/126858, WO 2016/200782 and WO 2017/015560).

Bispecific molecules binding to both PD-1 and LAG-3 (“PD-1 X LAG-3 bispecific molecules”) allows for great flexibility in the design and engineering in various applications, providing enhanced avidity to multimeric antigens, the cross-linking of differing antigens, and directed targeting to specific cell types relying on the presence of both target antigens. PD-1 X LAG-3 bispecific molecules for use in the treatment of cancer are described in PCT Publication Nos. WO 2015/200119, WO 2017/025498, WO 2018/083087, WO 2018/185043, WO 2018/134279, and WO 2018/217940. In particular, PD-1 X LAG-3 bispecific diabodies having novel PD-1 and LAG-3 binding domains and exemplary activity are described in WO 2017/019846.

### SUMMARY OF THE INVENTION

The present invention is in certain aspects directed to dosing regimens for administering a B7-H3-ADC for the treatment of cancer, particularly a cancer associated with expression of B7-H3. The invention in certain aspects concerns the use of such B7-H3-ADC optionally in combination with a PD-1 binding molecule for the treatment of cancer. Certain B7-H3-ADC and their uses in the treatment of cancer are described, for example, in PCT Publication No. WO 2017/180813. The invention in certain concerns the use of such B7-H3-ADC and an anti-PD-1 antibody, or a PD-1 X LAG-3 bispecific binding molecule. The dosing regimens for administering a B7-H3-ADC for the treatment of cancer, or a B7-H3-ADC in combination with a PD-1 binding molecule for the treatment of cancer, can include administration at regular dosing intervals or intermittent dosing intervals. In the dosing regimens where the B7-H3-ADC is administered in combination with a PD-1 binding molecule, their administration can be simultaneous or sequential in any order. The invention in certain aspects is directed to the use of such molecules, and to the use of pharmaceutical compositions and pharmaceutical kits that contain such molecules and that facilitate the use of such dosing regimens in the treatment of cancer.

In detail, the invention provides a method of treating a cancer comprising administering a B7-H3-ADC to a subject in need thereof, wherein said method comprises administering a B7-H3-ADC to a subject at a dose of about 0.5 mg/kg to about 5 mg/kg once about every 3 weeks.

The invention further provides a method of treating a cancer comprising administering a B7-H3-ADC to a subject in need thereof, wherein said method comprises administering a B7-H3-ADC to a subject at a dose of about 3 mg/kg to about 5 mg/kg once about every 3 weeks.

The invention further provides a method of treating a cancer comprising administering a B7-H3-ADC to a subject in need thereof, wherein said method comprises administering a B7-H3-ADC to a subject at a dose of 3 mg/kg to about 4 mg/kg once about every 3 weeks.

The invention further provides a method of treating a cancer comprising administering a B7-H3-ADC to a subject in need thereof, wherein said method comprises administering a B7-H3-ADC to a subject at a dose of about 4 mg/kg to about 5 mg/kg once about every 3 weeks.

The invention further provides the embodiment of such method (i.e., “the embodiment of such method” meaning an embodiment of an applicable method described herein) comprising administering a B7-H3-ADC to a subject in need thereof, wherein said method comprises administering a B7-H3-ADC to a subject at a dose of about 0.5 mg/kg to about 5 mg/kg once about every 4 weeks.

The invention further provides a method of treating a cancer comprising administering a B7-H3-ADC to a subject in need thereof, wherein said method comprises administering a B7-H3-ADC to a subject at a dose of about 3 mg/kg to about 5 mg/kg once about every 4 weeks.

The invention further provides a method of treating a cancer comprising administering a B7-H3-ADC to a subject in need thereof, wherein said method comprises administering a B7-H3-ADC to a subject at a dose of 3 mg/kg to about 4 mg/kg once about every 4 weeks.

The invention further provides a method of treating a cancer comprising administering a B7-H3-ADC to a subject in need thereof, wherein said method comprises administering a B7-H3-ADC to a subject at a dose of about 4 mg/kg to about 5 mg/kg once about every 4 weeks.

The invention further provides the embodiment of such method comprising administering a B7-H3-ADC to a subject in need thereof, wherein said method comprises administering a B7-H3-ADC to a subject at a dose of about 0.5 mg/kg, about 1 mg/kg, about 1.5 mg/kg, about 2 mg/kg, about 2.25 mg/kg, about 2.5 mg/kg, about 2.75 mg/kg, about 3 mg/kg, about 3.25 mg/kg, about 3.5 mg/kg, about 3.75 mg/kg, about 4 mg/kg, about 4.25 mg/kg, about 4.5 mg/kg, about 4.75 mg/kg or about 5 mg/kg.

The invention further provides the embodiment of such method comprising administering to a subject in need thereof:
(A) a B7-H3-ADC; and
(B) a PD-1 binding molecule,
   wherein the method comprises administering a B7-H3-ADC to the subject at a dose of about 0.5 mg/kg to about 5 mg/kg once every 3 weeks.

The invention further provides the embodiment of such method comprising administering to a subject in need thereof:
(A) a B7-H3-ADC; and
(B) a PD-1 binding molecule,
   wherein the method comprises administering a B7-H3-ADC to the subject at a dose of about 0.5 mg/kg to about 5 mg/kg once every 4 weeks.

The invention further provides the embodiment of such method wherein a B7-H3-ADC is represented by the formula:

Ab-(LM)ₘ-(D)ₙ,
wherein:
Ab is a humanized B7-H3 antibody or B7-H3 binding fragment thereof that binds to B7-H3 and comprises:
(i) the CDRL1 sequence RASESIYSYLA (SEQ ID NO: 39), the CDRL2 sequence NTKTLPE (SEQ ID NO: 40) and the CDRL3 sequence QHHYGTPPWT (SEQ ID NO: 41) in its Variable Light Chain (VL) domain, and
(ii) the CDRH1 sequence SYGMS (SEQ ID NO: 42), the CDRH2 sequence TINSGGSNTYY PDSLKG (SEQ ID NO: 43) and the CDRH3 sequence HDGGAMDY (SEQ ID NO: 44) in its Variable Heavy Chain (VH) domain;
D is a cytotoxic duocarmycin moiety;
LM comprises at least one bond or a Linker Molecule that covalently links Ab and D;
m is an integer between 0 and n and denotes the number of bonds or

Linker Molecules of the B7-H3-ADC, except when LM is a bond, m is not 0;
and
n is an integer between 1 and 10 and denotes the number of cytotoxic duocarmycin moieties covalently linked to the B7-H3-ADC.

The invention further provides such B7-H3-ADCs, wherein the Linker Molecule is absent and LM is at least one bond (i.e., m≥1), and B7-H3-ADCs that possess more than one Linker Molecule LM (i.e., m is an integer from 2 through n), each of which Linker Molecule LM covalently links a cytotoxic duocarmycin drug moiety D to the Ab of such B7-H3-ADCs. The invention further provides such B7-H3-ADCs whose Ab are covalently linked to more than one Linker Molecule LM, wherein all such Linker Molecules are identical. The cytotoxic duocarmycin drug moieties D that are covalently linked to the Ab of such B7-H3-ADCs may all be identical or may include 2, 3, 4, or more non-identical cytotoxic duocarmycin drug moieties D. The invention further provides such B7-H3-ADCs whose Ab are covalently linked to more than one Linker Molecule LM, wherein all such Linker Molecules are not identical. The cytotoxic duocarmycin drug moieties D that are covalently linked to the Ab of such B7-H3-ADCs may all be identical or may include 2, 3, 4, or more non-identical cytotoxic duocarmycin drug moieties D.

The invention further provides the embodiment of such method, wherein the B7-H3-ADC comprises:
(I) the humanized VL Domain comprising the amino acid sequence of SEQ ID NO:17, and
(II) the humanized VH Domain comprising the amino acid sequence of SEQ ID NO:18.

The invention further provides the embodiment of such method, wherein the Ab is an antibody.

The invention further provides the embodiment of such method, wherein the Ab further comprises an Fc Domain of a human IgG1.

The invention further provides the embodiment of such method, wherein the B7-H3-ADC comprises a Light Chain comprising the amino acid sequence of SEQ ID NO:19 and a Heavy Chain comprising the amino acid sequence of SEQ ID NO:20.

The invention further provides the embodiment of such method, wherein at least one of the LM is a Linker Molecule, and particularly wherein the LM Linker Molecule is a peptidic linker and/or a cleavable linker.

The invention further provides the embodiment of such method, wherein the peptidic linker is a valine-citrulline dipeptide linker.

The invention further provides the embodiment of such method, wherein the LM Linker Molecule further comprises a self-eliminating spacer between the cleavable linker and D.

The invention further provides the embodiment of such method, wherein the self-eliminating spacer comprises a para-aminobenzyloxycarbonyl moiety.

The invention further provides the embodiment of such method, wherein the LM further comprises a maleimide linker moiety between the cleavable linker and Ab.

The invention further provides the embodiment of such method, wherein LM is represented by the formula:

[V-(W)ₖ-(X)₁-A]

whereby the B7-H3-ADC is represented by the formula:

Ab-[V-(W)ₖ-(X)₁-A]-D
wherein:
V is a cleavable linker,
(W)ₖ-(X)₁-A is an elongated, self-eliminating spacer system, that self-eliminates via a l, (4+2n)-elimination,
W and X are each a l, (4+2n) electronic cascade spacer, being the same or different,
A is either a spacer group of formula (Y)ₘ, wherein Y is a l, (4+2n) electronic cascade spacer, or a group of formula U, being a cyclisation elimination spacer,
k, l and m are independently an integer of 0 (included) to 5 (included),
n is an integer of 0 (included) to 10 (included),
with the provisos that:
   when A is (Y)ₘ: then k+l+m≥1, and
   if k+l+m=1, then n>l;
   when A is U: then k+l≥1.
W, X, and Y are independently selected from compounds having the formula:

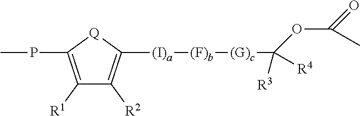

or the formula:

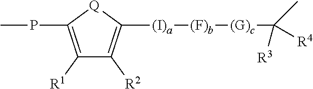

wherein: Q is —R⁵C═CR⁶—, S, O, NR^{S}, —R⁵C═N—, or —N═CR⁵—
   P is NR⁷, O or S
   a, b, and c are independently an integer of 0 (included) to 5 (included);
   I, F and G are independently selected from compounds having the formula:

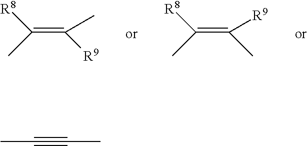

wherein R¹, R², R³, R⁴, R⁵, R⁶, R⁷, R⁸, and R⁹ independently represent H, C₁₋₆ alkyl, C₃₋₂₀ heterocyclyl, C₅₋₂₀ aryl, C₁₋₆ alkoxy, hydroxy (OH), amino (NH2), mono-substituted amino (NRₓH), di-substituted amino (NRₓ¹Rₓ²), nitro (NO₂), halogen, CF₃, CN, CONH₂, SO₂Me, CONHMe, cyclic C₁₋₅ alkylamino, imidazolyl, C₁₋₆ alkylpiperazinyl, morpholino, thiol (SH), thioether (SRₓ), tetrazole, carboxy (COOH), carboxylate (COORₓ), sulphoxy (S(═O)₂OH), sulphonate (S(═O)₂ORₓ), sulphonyl (S(═O)₂Rₓ), sulphixy (S(═O)OH), sulphinate (S(═O)ORₓ), sulphinyl (S(═O)Rₓ), phosphonooxy (OP(═O)(OH)₂), and phosphate (OP(═O)(ORₓ)₂), where Rₓ, Rₓ¹ and Rₓ² are independently selected from a C₁₋₆ alkyl group, a C₃₋₂₀ heterocyclyl group or a C₅₋₂₀ aryl group, two or more of the substituents R¹, R², R³, R⁴, R⁵, R⁶, R⁷, R⁸, or R⁹ optionally being connected to one another to form one or more aliphatic or aromatic cyclic structures;
U is selected from compounds having the formula:

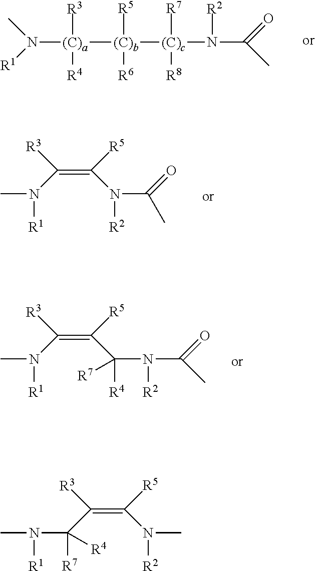

wherein:
a, b and c are independently selected to be an integer of 0 or 1;
   provided that a+b+c=2 or 3;
R¹ and/or R² independently represent H, C₁₋₆ alkyl, the alkyl being optionally substituted with one or more of the following groups: hydroxy (OH), ether (ORₓ), amino (NH₂), mono-substituted amino (NRₓH), disubstituted amino (NRₓ¹Rₓ²), nitro (NO₂), halogen, CF₃, CN, CONH₂, SO₂Me, CONHMe, cyclic C₁₋₅ alkylamino, imidazolyl, C₁₋₆ alkylpiperazinyl, morpholino, thiol (SH), thioether (SR_{X}), tetrazole, carboxy (COOH), carboxylate (COORₓ), sulphoxy (S(═O)₂OH), sulphonate (S(═O)₂OR_{X}), sulphonyl (S(═O)₂Rₓ), sulphixy (S(═O)OH), sulphinate (S(═O)ORx), sulphinyl (S(═O)Rx), phosphonooxy (OP(═O)(OH)₂), and phosphate (OP(═O)(ORₓ)₂), where Rₓ, Rₓ¹ and Rₓ² are selected from a C₁₋₆ alkyl group, a C₃₋₂₀ heterocyclyl group or a C₅₋₂₀ aryl group; and
R³, R⁴, R⁵, R⁶, R⁷ and R⁸ independently represent H, C₁₋₆ alkyl, C₃₋₂₀ heterocyclyl, C₅₋₂₀ aryl, C₁₋₆ alkoxy, hydroxy (OH), amino (NH₂), mono-substituted amino (NRₓH), disubstituted amino (NRₓ¹Rₓ²), nitro (NO₂), halogen, CF₃, CN, CONH₂, SO₂Me, CONHMe, cyclic C₁₋₅ alkylamino, imidazolyl, C₁₋₆ alkylpiperazinyl, morpholino, thiol (SH), thioether (SRₓ), tetrazole, carboxy (COOH), carboxylate (COORₓ), sulphoxy (S(═O)₂OH), sulphonate (S(═O)₂ORₓ), sulphonyl (S(═O)₂Rₓ), sulphixy (S(═O)OH), sulphinate (S(═O)ORₓ), sulphinyl (S(═O)Rₓ), phosphonooxy (OP(═O)(OH)2), and phosphate (OP(═O)(ORₓ)₂), where Rₓ, Rₓ¹ and Rₓ² are selected from a C₁₋₆ alkyl group, a C₃₋₂₀ heterocyclyl group or a C₅₋₂₀ aryl group, and two or more of the substituents R², R³, R⁴, R⁵, R⁶, R⁷, or R⁸ are optionally connected to one another to form one or more aliphatic or aromatic cyclic structures.

The invention further provides the embodiment of such method, wherein the LM Linker Molecule comprises:
(1) p-aminobenzyloxycarbonyl-p-aminobenzyloxycarbonyl;
(2) p-aminobenzyloxycarbonyl-p-aminobenzyloxycarbonyl-p-aminobenzyloxycarbonyl;
(3) p-ammocinnamyloxycarbonyl;
(4) p-aminocinnamyloxycarbonyl-p-aminobenzyloxycarbonyl;
(5) p-amino-benzyloxycarbonyl-p-aminocinnamyloxycarbonyl;
(6) p-aminocinnamyloxycarbonyl-p-aminocinnamyloxycarbonyl;
(7) p-aminophenylpentadienyloxycarbonyl;
(8) p-aminophenylpentadienyloxycarbonyl-p-aminocinnamyloxycarbonyl;
(9) p-aminophenylpentadienyloxycarbonyl-paminobenzyloxycarbonyl;
(10) p-aminophenylpentadienyloxycarbonyl-p-aminophenylpentadienyloxycarbonyl;
(11) p-aminobenzyloxycarbonyl(methylamino)ethyl(methylamino) carbonyl;
(12) p-aminocinnamyloxycarbonyl(methylamino)ethyl(methylamino) carbonyl;
(13) p-aminobenzyloxycarbonyl-p-aminobenzyloxycarbonyl(methylamino) ethyl(methylamino)carbonyl;
(14) p-aminocinnamyloxycarbonyl-p-aminobenzyloxycarbonyl (methylamino)ethyl(methylamino)carbonyl;
(15) p-aminobenzyloxycarbonyl-p-aminocinnamyloxycarbonyl (methylamino)ethyl(methylamino)-carbonyl;
(16) p-aminocinnamyloxycarbonyl-p-aminocinnamyloxycarbonyl (methylamino)ethyl(methylamino)carbonyl;
(17) p-aminobenzyloxycarbonyl-p-aminobenzyl;
(18) p-aminobenzyloxycarbonyl-p-aminobenzyloxycarbonyl -p-aminobenzyl;
(19) p-aminocinnamyl;
(20) p-aminocinnamyloxycarbonyl-p-aminobenzyl;
(21) p-aminobenzyloxycarbonyl-p-aminocinnamyl;
(22) p-amino-cinnamyloxycarbonyl-p-aminocinnamyl;
(23) p-aminophenylpentadienyl;
(24) p-aminophenylpentadienyloxycarbonyl-p-aminocinnamyl;
(25) p-aminophenylpentadienyloxycarbonyl-p-aminobenzyl; or
(26) p-aminophenylpentadienyloxycarbonyl-p-aminophenylpentadienyl.

The invention further provides the embodiment of such method wherein the LM Linker Molecule is conjugated to the side chain of an amino acid of a polypeptide chain of Ab and binds the Ab to a molecule of the cytotoxic duocarmycin moiety D.

The invention further provides the embodiment of such method wherein the cytotoxic duocarmycin moiety D comprises a duocarmycin cytotoxin selected from the group consisting of duocarmycin A, duocarmycin B1, doucarmycin B2, duocarmycin C1, duocarmycin C2, duocarmycin D, duocarmycin SA, CC-1065, adozelesin, bizelesin, carzelesin (U-80244) and spiro-duocarmycin (DUBA)).

The invention further provides the embodiment of such method, wherein the cytotoxic duocarmycin moiety D comprises seco-duocarmycin.

The invention further provides the embodiment of such method, wherein the LM Linker Molecule is covalently linked to the Ab via reduced inter-chain disulfides.

The invention further provides the embodiment of such method, wherein a B7-H3-ADC is administered at a dose of about 2 mg/kg.

The invention further provides the embodiment of such method, wherein a B7-H3-ADC is administered at a dose of about 2.25 mg/kg.

The invention further provides the embodiment of such method, wherein a B7-H3-ADC is administered at a dose of about 2.5 mg/kg.

The invention further provides the embodiment of such method, wherein a B7-H3-ADC is administered at a dose of about 2.75 mg/kg.

The invention further provides the embodiment of such method, wherein a B7-H3-ADC is administered at a dose of about 3 mg/kg.

The invention further provides the embodiment of such method, wherein a B7-H3-ADC is administered at a dose of about 3.25 mg/kg.

The invention further provides the embodiment of such method, wherein a B7-H3-ADC is administered at a dose of about 3.5 mg/kg.

The invention further provides the embodiment of such method, wherein a B7-H3-ADC is administered at a dose of about 3.75 mg/kg.

The invention further provides the embodiment of such method, wherein a B7-H3-ADC is administered at a dose of about 4 mg/kg.

The invention further provides the embodiment of such method, wherein a B7-H3-ADC is administered at a dose of about 4.25 mg/kg.

The invention further provides the embodiment of such method, wherein a B7-H3-ADC is administered at a dose of about 4.5 mg/kg.

The invention further provides the embodiment of such method, wherein a B7-H3-ADC is administered at a dose of about 4.75 mg/kg.

The invention further provides the embodiment of such method, wherein a B7-H3-ADC is administered at a dose of about 5 mg/kg.

The invention further provides the embodiment of such method, wherein a B7-H3-ADC is administered by intravenous (IV) infusion over a period of about 60 minutes.

The invention further provides the embodiment of such method, wherein a B7-H3-ADC is administered in combination with a therapeutically effective dose of a PD-1 binding molecule.

The invention further provides the embodiment of such method, wherein the PD-1 binding molecule is selected from the group consisting of an antibody, a single chain antibody, an Fab fragment, an F(ab′)2 fragment, an Fab′ fragment, an Fsc fragment, an Fv fragment, an scFv, an sc(Fv)2, and a diabody.

The invention further provides the embodiment of such method, wherein the PD-1 binding molecule is selected from the group consisting of hPD-1 mAb-A, pembrolizumab, nivolumab and PD-1 X LAG-3 BD.

The invention further provides the embodiment of such method, wherein the PD-1 binding molecule is hPD-1 mAb-A or PD-1 x LAG-3 BD.

The invention further provides the embodiment of such method, wherein the PD-1 binding molecule comprises a variable heavy (VH) domain comprising VH complementarity determining region (CDR)1, VH CDR2 and VH CDR3, wherein
the VH CDR1 comprises the amino acid sequence SYWMN (SEQ ID NO:23);
the VH CDR2 comprises the amino acid sequence VIHPSDSETWLDQKFKD (SEQ ID NO:24);
the VH CDR3 comprises the amino acid sequence EHYGTSPFAY (SEQ ID NO:25); and
wherein the antibody comprises a variable light (VL) domain comprising VL CDR1, VL CDR2, and VL CDR3, wherein:
the VL CDR1 comprises the amino acid sequence RASESVDNYGMSFMNW (SEQ ID NO:26);
the VL CDR2 comprises the amino acid sequence AASNQGS (SEQ ID NO:27); and
the VL CDR3 comprises the amino acid sequence QQSKEVPYT (SEQ ID NO:28).

The invention further provides the embodiment of such method, wherein the VH domain of the PD-1 binding molecule comprises the amino acid sequence set forth in SEQ ID NO:32 and said VL domain comprises the amino acid sequence set forth in SEQ ID NO:31.

The invention further provides the embodiment of such method, wherein the PD-1 binding molecule is hPD-1 mAb-A.

The invention further provides the embodiment of such method, wherein the method comprises administering hPD-1 mAb-A once about every 3 weeks at a flat dose selected from the group consisting of about 375 mg, about 500 mg, and about 750 mg.

The invention further provides the embodiment of such method, wherein the method comprises administering hPD-1 mAb-A once about every 4 weeks at a flat dose selected from the group consisting of about 375 mg, about 500 mg, and about 750 mg.

The invention further provides the embodiment of such method, wherein hPD-1 mAb-A is administered once about every 3 weeks at a flat dose of about 375 mg.

The invention further provides the embodiment of such method, wherein hPD-1 mAb-A is administered once about every 3 weeks at a flat dose of about 500 mg.

The invention further provides the embodiment of such method, wherein a B7-H3-ADC is administered at a dose of about 3 mg/kg and hPD-1 mAb-A is administered at a flat dose of about 375 mg once every 3 weeks.

The invention further provides the embodiment of such method, wherein a B7-H3-ADC is administered at a dose of about 3.25 mg/kg and hPD-1 mAb-A is administered at a flat dose of about 375 mg once every 3 weeks.

The invention further provides the embodiment of such method, wherein a B7-H3-ADC is administered at a dose of about 3.5 mg/kg and hPD-1 mAb A is administered at a flat dose of about 375 mg once every 3 weeks.

The invention further provides the embodiment of such method, wherein a B7-H3-ADC is administered at a dose of about 3.75 mg/kg and hPD-1 mAb A is administered at a flat dose of about 375 mg once every 3 weeks.

The invention further provides the embodiment of such method, wherein a B7-H3-ADC is administered at a dose of about 4 mg/kg and hPD-1 mAb-A is administered at a flat dose of about 375 mg once every 3 weeks.

The invention further provides the embodiment of such method, wherein a B7-H3-ADC is administered at a dose of about 4.25 mg/kg and hPD-1 mAb-A is administered at a flat dose of about 375 mg once every 3 weeks.

The invention further provides the embodiment of such method, wherein a B7-H3-ADC is administered at a dose of about 4.5 mg/kg and hPD-1 mAb-A is administered at a flat dose of about 375 mg once every 3 weeks.

The invention further provides the embodiment of such method, wherein a B7-H3-ADC is administered at a dose of about 4.75 mg/kg and hPD-1 mAb A is administered at a flat dose of about 375 mg once every 3 weeks.

The invention further provides the embodiment of such method, wherein a B7-H3-ADC is administered at a dose of about 5 mg/kg and hPD-1 mAb-A is administered at a flat dose of about 375 mg once every 3 weeks.

The invention further provides the embodiment of such method, wherein a B7-H3-ADC is administered at a dose of about 3 mg/kg and hPD-1 mAb-A is administered at a flat dose of about 375 mg once every 4 weeks.

The invention further provides the embodiment of such method, wherein a B7-H3-ADC is administered at a dose of about 3.25 mg/kg and hPD-1 mAb-A is administered at a flat dose of about 375 mg once every 4 weeks.

The invention further provides the embodiment of such method, wherein a B7-H3-ADC is administered at a dose of about 3.5 mg/kg and hPD-1 mAb-A is administered at a flat dose of about 375 mg once every 4 weeks.

The invention further provides the embodiment of such method, wherein a B7-H3-ADC is administered at a dose of about 3.75 mg/kg and hPD-1 mAb-A is administered at a flat dose of about 375 mg once every 4 weeks.

The invention further provides the embodiment of such method, wherein a B7-H3-ADC is administered at a dose of about 4 mg/kg and hPD-1 mAb-A is administered at a flat dose of about 375 mg once every 4 weeks.

The invention further provides the embodiment of such method, wherein a B7-H3-ADC is administered at a dose of about 4.25 mg/kg and hPD-1 mAb-A is administered at a flat dose of about 375 mg once every 4 weeks.

The invention further provides the embodiment of such method, wherein a B7-H3-ADC is administered at a dose of about 4.5 mg/kg and hPD-1 mAb-A is administered at a flat dose of about 375 mg once every 4 weeks.

The invention further provides the embodiment of such method, wherein a B7-H3-ADC is administered at a dose of about 4.75 mg/kg and hPD-1 mAb-A is administered at a flat dose of about 375 mg once every 4 weeks.

The invention further provides the embodiment of such method, wherein a B7-H3-ADC is administered at a dose of about 5 mg/kg and hPD-1 mAb-A is administered at a flat dose of about 375 mg once every 4 weeks.

The invention further provides the embodiment of such method, wherein hPD-1 mAb-A is administered by IV infusion over a period of about 60 minutes.

The invention further provides the embodiment of such method, wherein the antibody that binds to human PD-1 is pembrolizumab.

The invention further provides the embodiment of such method, wherein pembrolizumab is administered once about every 3 weeks at a flat dose of about 200 mg.

The invention further provides the embodiment of such method, wherein pembrolizumab is administered by IV infusion over a period of about 30 minutes.

The invention further provides the embodiment of such method, wherein the PD-1 binding molecule is nivolumab.

The invention further provides the embodiment of such method, wherein nivolumab is administered once about every 2 weeks at a flat dose of about 240 mg.

The invention further provides the embodiment of such method, wherein nivolumab is administered once about every 4 weeks at a flat dose of about 480 mg.

The invention further provides the embodiment of such method, wherein nivolumab is administered by IV infusion over a period of about 30 minutes.

The invention further provides the embodiment of such method, wherein the PD-1 binding molecule is PD-1 X LAG-3 BD.

The invention further provides the embodiment of such method, wherein PD-1 X LAG-3 BD comprises two polypeptide chains that comprise the amino acid sequence of SEQ ID NO:37 and two polypeptide chains that comprises the amino acid sequence of SEQ ID NO:38.

The invention further provides the embodiment of such method, wherein PD-1 X LAG-3 BD is administered at a flat dose of about 300 mg once every 2 weeks.

The invention further provides the embodiment of such method, wherein PD-1 X LAG-3 BD is administered at a flat dose of about 300 mg once every 3 weeks.

The invention further provides the embodiment of such method, wherein PD-1 X LAG-3 BD is administered at a flat dose of about 600 mg once every 2 weeks.

The invention further provides the embodiment of such method, wherein PD-1 X LAG-3 BD is administered at a flat dose of about 600 mg once every 3 weeks.

The invention further provides the embodiment of such method, wherein PD-1 X LAG-3 BD is administered by IV infusion over a period of 30-240 minutes.

The invention further provides the embodiment of such method, wherein PD-1 X LAG-3 BD is administered by IV infusion over a period of about 30-90 minutes.

The invention further provides the embodiment of such method, wherein a B7-H3-ADC and hPD-1 mAb-A are administered sequentially to a subject in separate pharmaceutical compositions.

The invention further provides the embodiment of such method, wherein the pharmaceutical composition comprising hPD-1 mAb-A is administered before the administration of the pharmaceutical composition comprising a B7-H3-ADC.

The invention further provides the embodiment of such method, wherein a B7-H3-ADC and pembrolizumab are administered sequentially to a subject in separate pharmaceutical compositions.

The invention further provides the embodiment of such method, wherein a B7-H3-ADC and nivolumab are administered sequentially to a subject in separate pharmaceutical compositions.

The invention further provides the embodiment of such method, wherein a B7-H3-ADC and PD-1 X LAG-3 BD are administered sequentially to a subject in separate pharmaceutical compositions.

The invention further provides the embodiment of such method, wherein a B7-H3-ADC is provided in a pharmaceutical kit that comprises:
(A) a pharmaceutical composition comprising from about 0.5 mg/ml to about 5 mg/ml of a B7-H3-ADC; and
(B) an instructional material,
   wherein the instructional material instructs that the pharmaceutical composition comprising a B7-H3-ADC is to be administered optionally in combination with a pharmaceutical composition comprising a PD-1 binding molecule.
   The invention further provides the embodiment of such method, wherein the PD-1 binding molecule is hPD-1 mAb-A, pembrolizumab, nivolumab or PD-1 X LAG-3 BD.

The invention further provides the embodiment of such method, wherein a B7-H3-ADC is provided in a pharmaceutical kit wherein the B7-H3-ADC comprises:
(I) the humanized VL Domain comprising the amino acid sequence of SEQ ID NO:17, and
(II) the humanized VH Domain comprising the amino acid sequence of SEQ ID NO:18.

The invention further provides the embodiment of such method, wherein the instructional manual of such pharmaceutical kit instructs that a B7-H3-ADC is administered at a dose of about 0.5 mg/kg, about 1 mg/kg, about 1.5 mg/kg, about 2 mg/kg, about 2.25 mg/kg, about 2.5 mg/kg, about 2.75 mg/kg, about 3 mg/kg, about 3.25 mg/kg, about 3.5 mg/kg, about 3.75 mg/kg, about 4 mg/kg, about 4.25 mg/kg, about 4.5 mg/kg, about 4.75 mg/kg or about 5 mg/kg.

The invention further provides the embodiment of such method, wherein the instructional manual of such pharmaceutical kit instructs that hPD-1 mAb-A is administered at a flat dose of about 375 mg or about 500 mg once every 3 weeks.

The invention further provides the embodiment of such method, wherein the instructional manual of such pharmaceutical kit instructs that pembrolizumab is administered with a flat dose of about 200 mg of once every 3 weeks.

The invention further provides the embodiment of such method, wherein the instructional manual of such pharmaceutical kit instructs that PD-1 X LAG-3 BD is administered at a flat dose of about 300 mg or about 600 mg once every 2 weeks or once every 3 weeks.

The invention further provides the embodiment of such method, wherein the instructional manual of such pharmaceutical kit instructs that a B7-H3-ADC and hPD-1 mAb-A are administered by IV infusion over a period of about 60 minutes.

The invention further provides the embodiment of such method, wherein the instructional manual of such pharmaceutical kit instructs that a B7-H3-ADC is administered by IV infusion over a period of about 60 minutes and PD-1 X LAG-3 BD is administered by IV infusion over a period of about 30-90 minutes.

The invention further provides the embodiment of such method, wherein the instructional manual of such pharmaceutical kit instructs that a B7-H3-ADC is administered by IV infusion over a period of about 60 minutes and PD-1 X LAG-3 BD is administered by IV infusion over a period of about 30-240 minutes.

The invention further provides the embodiment of such method, wherein a B7-H3-ADC is to be administered in combination with the PD-1 binding molecule for the treatment of a cancer in which B7-H3 is expressed.

The invention further provides the embodiment of such method, wherein the cancer is selected from the group consisting of: an adrenal gland cancer, an AIDS-associated cancer, an alveolar soft part sarcoma, an astrocytic tumor, an anal cancer (e.g., squamous cell carcinoma of the anal canal (SCAC)), a bladder cancer, a bone cancer, a brain and spinal cord cancer, a metastatic brain tumor, a B-cell cancer, a breast cancer (e.g., a HER2+ breast cancer or triple negative breast cancer (TNBC)), a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, a gastric cancer, a gestational trophoblastic disease, a germ cell tumor, a head and neck cancer, a glioblastoma, a hematological malignancy, a hepatocellular carcinoma, an islet cell tumor, a Kaposi's Sarcoma, a kidney cancer, a leukemia (e.g., an acute myeloid leukemia), a liposarcoma/malignant lipomatous tumor, a liver cancer, a lymphoma, a lung cancer (e.g., a non-small-cell lung cancer (NSCLC)), a medulloblastoma, a melanoma, a meningioma, a mesothelioma pharyngeal cancer, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a pheochromocytoma, a pituitary tumor, a prostate cancer (e.g., a metastatic castration resistant prostate cancer (mCRPC)), a posterior uveal melanoma, a renal metastatic cancer, a rhabdoid tumor, a rhabdomyosarcoma, a sarcoma, a skin cancer, a small round blue cell tumor of childhood (e.g, neuroblastoma or rhabdomyosarcoma), a soft-tissue sarcoma, a squamous cell cancer (e.g., a squamous cell cancer of the head and neck (SCCHN), a stomach cancer, a synovial sarcoma, a testicular cancer, a thymic carcinoma, a thymoma, a thyroid cancer (e.g., a thyroid metastatic cancer), and a uterine cancer.

The invention further provides the embodiment of such method, wherein the cancer is a prostate cancer, an anal cancer, a squamous cell cancer, a breast cancer, a melanoma, or a lung cancer.

The invention further provides the embodiment of such method, wherein the cancer is mCRPC, SCAC, SCCHN, TNBC, uveal melanoma, or NSCLC.

The invention further provides the embodiment of such method, further comprising administering a therapeutically or prophylactically effective amount of one or more additional therapeutic agents or chemotherapeutic agents.

The invention further provides the embodiment of such method, wherein the chemotherapeutic agent is a platinum-based chemotherapeutic agent.

The invention further provides the embodiment of such method, wherein the chemotherapeutic agent is a taxane.

The invention further provides the embodiment of such method, wherein the subject in need thereof is a human.

### BRIEF DESCRIPTION OF THE DRAWINGS

FIG. 1 provides a schematic showing representative covalently bonded tetravalent diabodies, such as a PD-1 X LAG-3 BD, having four epitope-binding sites composed of two pairs of polypeptide chains (i.e., four polypeptide chains in all). One polypeptide of each pair has an E-coil Heterodimer-Promoting Domain and the other polypeptide of each pair has a K-coil Heterodimer-Promoting Domain. As shown, a cysteine residue may be present in a linker and/or in the Heterodimer-Promoting Domain. One polypeptide of each pair possesses a linker comprising a cysteine (which linker may comprise all or a portion of a hinge region) and CH2 and/or CH3 Domain, such that the associated chains form all or part of an Fc Region. VL and VH Domains that recognize the same epitope are shown using the same shading or fill pattern. The VL and VH Domains recognize different epitopes and the resulting molecule possesses four epitope-binding sites and is bispecific and bivalent with respect to each bound epitope.

FIG. 2 shows the results of a study of the ability of a B7-H3-ADC of the present invention, in combination with anti-PD-1 antibody (RMP1-14), to mediate in vivo cytotoxicity against subcutaneously implanted MC38/hB7-H3 (murine colorectal cancer tumor cells overexpressing human B7-H3) in a C57BL/6 syngeneic mouse model. B7-H3-ADC was administered on day 15. Anti-PD-1 antibody was administered on days 15, 18, 21, 23, 25, 28, 30, 32, 35, and 37. Vehicle was administered on day 15. The tumor growth curves are presented for mice treated intraperitoneally with 5 mg/kg or 10 mg/kg B7-H3-ADC alone, 20 mg/kg of anti-PD-1 antibody alone, a combination of 5 mg/kg B7-H3-ADC+20 mg/kg anti-PD-1 antibody, a combination of 10 mg/kg B7-H3-ADC+20 mg/kg anti-PD-1 antibody, or vehicle alone.

FIG. 3 shows the results of a study of the ability of a B7-H3-ADC of the present invention, in combination with anti-PD-1 antibody (RMP1-14), to mediate in vivo cytotoxicity against subcutaneously implanted CT26/hB7-H3 (murine colorectal cancer tumor cells overexpressing human B7-H3) in a BALB/c syngeneic mouse model. B7-H3-ADC was administered on day 13. Anti-PD-1 antibody was administered on days 13, 16, 19, 22, 26, 29, 33, and 36. Vehicle was administered on day 13. The tumor growth curves are presented for mice treated intraperitoneally with 10 mg/kg B7-H3-ADC alone, 20 mg/kg of anti-PD-1 antibody alone, a combination of 10 mg/kg B7-H3-ADC+20 mg/kg anti-PD-1 antibody, or vehicle alone.

FIG. 4 shows a waterfall plot of the percent of change of target lesions (plotted as % change from baseline) among response-evaluable cohort escalation and cohort expansion patients by tumor type and by dose. Patients were treated with 0.5 mg/kg, 1 mg/kg, 2 mg/kg, 3 mg/kg, or 4 mg/kgB7-H3-ADC in cohort escalation or with 3.0 mg/kg B7-H3-ADC in cohort expansion. The dotted lines indicate a change from baseline of 20% or −30%. Abbreviations: CRC=colorectal carcinoma; NSCLC=non-small cell lung cancer; SCLC=small cell lung cancer; mCRPC=metastatic castration-resistant prostate cancer.

FIGS. 5A and 5B present images of a target lung lesion in one non-small cell lung cancer (NSCLC) patient at baseline (FIG. 5A) and at week 6 following 2 Q3W doses of 2 mg/kg B7-H3-ADC (FIG. 5B). The lesion is noted in each image by the white arrow.

FIG. 6 shows a waterfall plot of the percent of change of prostate specific antigen (PSA; plotted as % change from baseline) among response-evaluable cohort escalation and cohort expansion mCRPC patients by dose. Patients were treated with 2.0 mg/kg, 3.0 mg/kg or 4.0 mg/kg B7-H3-ADC in cohort escalation or with 3.0 mg/kg B7-H3-ADC in cohort expansion. The dotted lines indicate a change from baseline of 25% or −50%.

### DETAILED DESCRIPTION OF THE INVENTION

The present invention is directed to dosing regimens for administering a B7-H3-ADC for the treatment of cancer, particularly a cancer associated with expression of B7-H3. Certain B7-H3-ADC and their uses in the treatment of cancer are described, for example, in PCT Publication No. WO 2017/180813, expressly incorporated by reference herein. The invention particularly concerns the use of such B7-H3-ADC optionally in combination with a PD-1 binding molecule for the treatment of cancer. The invention particularly concerns the use of a B7-H3-ADC and an anti-PD-1 antibody, or a PD-1 X LAG-3 bispecific molecule. The dosing regimens for administering a B7-H3-ADC for the treatment of cancer, or a B7-H3-ADC in combination with a PD-1 binding molecule for the treatment of cancer, can include administration at regular dosing intervals or intermittent dosing intervals. In the dosing regimens where the B7-H3-ADC is administered in combination with a PD-1 binding molecule, their administration can be simultaneous or sequential in any order. The invention is directed to the use of such molecules, and to the use of pharmaceutical compositions and pharmaceutical kits that contain such molecules and that facilitate the use of such dosing regimens in the treatment of cancer.

I. Antibodies and Their Binding Domains

The antibodies of the present invention are immunoglobulin molecules capable of specific binding to a target, such as a carbohydrate, polynucleotide, lipid, polypeptide, etc., through at least one antigen recognition site, located in the Variable Domain of the immunoglobulin molecule. A B7-H3-ADC of the present invention thus comprises an antibody that binds to B7-H3. As used herein, the terms “antibody” and “antibodies” refer to monoclonal antibodies, multispecific antibodies, human antibodies, humanized antibodies, synthetic antibodies, chimeric antibodies, polyclonal antibodies, camelized antibodies, single-chain Fvs (scFv), single-chain antibodies, Fab fragments, F(ab′) fragments, disulfide-linked bispecific Fvs (sdFv), intrabodies, and epitope-binding fragments of any of the above. In particular, the term “antibody” includes immunoglobulin molecules and immunologically active fragments of immunoglobulin molecules, i.e., molecules that contain an epitope-binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass. Antibodies are capable of “immunospecifically binding” to a polypeptide or protein or a non-protein molecule (or of binding to such molecule in an “immunospecific manner”) due to the presence on such molecule of a particular domain or moiety or conformation (an “epitope”). An epitope-containing molecule may have immunogenic activity, such that it elicits an antibody production response in an animal; such molecules are termed “antigens”.

As used herein, an antibody, diabody or other epitope-binding molecule is said to “immunospecifically” bind a region of another molecule (i.e., an epitope) if it reacts or associates more frequently, more rapidly, with greater duration and/or with greater affinity with that epitope relative to alternative epitopes. For example, an antibody that immunospecifically binds to a viral epitope is an antibody that binds this viral epitope with greater affinity, avidity, more readily, and/or with greater duration than it immunospecifically binds to other viral epitopes or non-viral epitopes. It is also understood by reading this definition that, for example, an antibody (or moiety or epitope) that immunospecifically binds to a first target may or may not specifically or preferentially bind to a second target. As such, “immunospecific binding” does not necessarily require (although it can include) exclusive binding. Generally, but not necessarily, reference to binding means “immunospecific” binding. Two molecules are said to be capable of binding to one another in a “physiospecific” manner, if such binding exhibits the specificity with which receptors bind to their respective ligands.

The term “monoclonal antibody” refers to a homogeneous antibody population wherein the monoclonal antibody is comprised of amino acids (naturally occurring or non-naturally naturally occurring) that are involved in the selective binding of an antigen. Monoclonal antibodies are highly specific, being directed against a single epitope (or antigenic site). The term “monoclonal antibody” encompasses not only intact monoclonal antibodies and full-length monoclonal antibodies, but also fragments thereof (such as Fab, Fab′, F(ab′)₂, Fv, etc.), single-chain (scFv) binding molecules, mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.). The term includes whole immunoglobulins as well as the fragments etc. described above under the definition of “antibody.” Methods of making monoclonal antibodies are known in the art. One method which may be employed is the method of Kohler, G. et al. (1975) “Continuous Cultures Of Fused Cells Secreting Antibody Of Predefined Specificity,” Nature 256:495-497 or a modification thereof. Typically, monoclonal antibodies are developed in mice, rats or rabbits. The antibodies are produced by immunizing an animal with an immunogenic amount of cells, cell extracts, or protein preparations that contain the desired epitope. The immunogen can be, but is not limited to, primary cells, cultured cell lines, cancerous cells, proteins, peptides, nucleic acids, or tissue. Alternatively, existing monoclonal antibodies and any other equivalent antibodies that are immunospecific for a desired pathogenic epitope can be sequenced and produced recombinantly by any means known in the art. In one embodiment, such an antibody is sequenced and the polynucleotide sequence is then cloned into a vector for expression or propagation. The sequence encoding the antibody of interest may be maintained in a vector in a host cell and the host cell can then be expanded and frozen for future use. The polynucleotide sequence of such antibodies may be used for genetic manipulation to generate the monospecific or multispecific (e.g., bispecific, trispecific and tetraspecific) molecules of the invention as well as an affinity optimized, a chimeric antibody, a humanized antibody, and/or a caninized antibody, to improve the affinity, or other characteristics of the antibody. The general principle in humanizing an antibody involves retaining the basic sequence of the antigen-binding portion of the antibody, while swapping the non-human remainder of the antibody with human antibody sequences.

Natural antibodies (such as IgG antibodies) are composed of two “Light Chains” complexed with two “Heavy Chains.” Each Light Chain contains a Variable Domain (“VL”) and a Constant Domain (“CL”). Each Heavy Chain contains a Variable Domain (“VH”), three Constant Domains (“CH1,” “CH2” and “CH3”), and a “Hinge” Region (“H”) located between the CH1 and CH2 Domains. The basic structural unit of naturally occurring immunoglobulins (e.g., IgG) is thus a tetramer having two light chains and two heavy chains, usually expressed as a glycoprotein of about 150,000 Da. The amino-terminal (“N-terminal”) portion of each chain includes a Variable Domain of about 100 to 110 or more amino acids primarily responsible for antigen recognition. The carboxy-terminal (“C-terminal”) portion of each chain defines a constant region, with light chains having a single Constant Domain and heavy chains usually having three Constant Domains and a Hinge Domain. Thus, the structure of the light chains of an IgG molecule is n-VL-CL-c and the structure of the IgG heavy chains is n-VH-CH1-H-CH2-CH3-c (where n and c represent, respectively, the N-terminus and the C-terminus of the polypeptide).

A. Characteristics of Antibody Variable Domains

The Variable Domains of an IgG molecule consist of the complementarity determining regions (“CDR”), which contain the residues in contact with epitope, and non-CDR segments, referred to as framework segments (“FR”), which in general maintain the structure and determine the positioning of the CDR loops so as to permit such contacting (although certain framework residues may also contact antigen). Thus, the VL and VH Domains have the structure n-FR1-CDR1-FR2-CDR2-FR3-CDR3-FR4-c. The amino acid sequences of the CDRs determine whether an antibody will be able to bind to a particular epitope. Interaction of an antibody light chain with an antibody heavy chain and, in particular, interaction of their VL and VH Domains, forms an epitope-binding site of the antibody.

Amino acids from the Variable Domains of the mature heavy and light chains of immunoglobulins are designated by the position of an amino acid in the chain. Kabat (SEQUENCES OF PROTEINS OF IMMUNOLOGICAL INTEREST, 5th Ed. Public Health Service, NH1, MD (1991)) described numerous amino acid sequences for antibodies, identified an amino acid consensus sequence for each subgroup, and assigned a residue number to each amino acid, and the CDRs and FRs are identified as defined by Kabat (it will be understood that CDR_{H}1 as defined by Chothia, C. & Lesk, A. M. ((1987) “Canonical structures for the hypervariable regions of immunoglobulins,” J. Mol. Biol. 196:901-917) begins five residues earlier). Kabat's numbering scheme is extendible to antibodies not included in his compendium by aligning the antibody in question with one of the consensus sequences in Kabat by reference to conserved amino acids. This method for assigning residue numbers has become standard in the field and readily identifies amino acids at equivalent positions in different antibodies, including chimeric or humanized variants. For example, an amino acid at position 50 of a human antibody light chain occupies the equivalent position to an amino acid at position 50 of a mouse antibody light chain. The positions within the VL and VH Domains at which the their CDRs commence and end are thus well defined and can be ascertained by inspection of the sequences of the VL and VH Domains (see, e.g., Martin, C. R. (2010) “Protein Sequence and Structure Analysis of Antibody Variable Domains,” In: ANTIBODY ENGINEERING VOL. 2 (Kontermann, R. and Dübel, S. (eds.), Springer-Verlag Berlin Heidelberg, Chapter 3 (pages 33-51)).

Polypeptides that are (or may serve as) the first, second and third CDR of the Light Chain of an antibody are herein respectively designated as: CDR_{L}1 Domain, CDR_{L}2 Domain, and CDR_{L}3 Domain. Similarly, polypeptides that are (or may serve as) the first, second and third CDR of the Heavy Chain of an antibody are herein respectively designated as: CDR_{H}1 Domain, CDR_{H}2 Domain, and CDR_{H}3 Domain. Thus, the terms CDR_{L}1 Domain, CDR_{L}2 Domain, CDR_{L}3 Domain, CDR_{H}1 Domain, CDR_{H}2 Domain, and CDR_{H}3 Domain are directed to polypeptides that when incorporated into a protein cause that protein to be able to bind to a specific epitope regardless of whether such protein is an antibody having light and heavy chains or is a diabody or a single-chain binding molecule (e.g., an scFv, a BiTe, etc.), or is another type of protein. Accordingly, as used herein, the term “epitope-binding fragment” denotes a fragment of a molecule capable of immunospecifically binding to an epitope. An epitope-binding fragment may contain any 1, 2, 3, 4, or 5 the CDR Domains of an antibody, or may contain all 6 of the CDR Domains of an antibody and, although capable of immunospecifically binding to such epitope, may exhibit an immunospecificity, affinity or selectivity toward such epitope that differs from that of such antibody. Preferably, however, an epitope-binding fragment will contain all 6 of the CDR Domains of such antibody. An epitope-binding fragment of an antibody may be a single polypeptide chain (e.g., an scFv), or may comprise two or more polypeptide chains, each having an amino terminus and a carboxy terminus (e.g., a diabody, a Fab fragment, an Fab₂ fragment, etc.). Unless specifically noted, the order of domains of the protein molecules described herein is in the “N-terminal to C-Terminal” direction.

The invention particularly encompasses single-chain Variable Domain fragments (“scFv”) comprising a humanized anti-B7-H3-VL and/or VH Domain of this invention. Single-chain Variable Domain fragments comprise VL and VH Domains that are linked together using a short “Linker” peptide. Such Linkers can be modified to provide additional functions, such as to permit the attachment of a drug or to permit attachment to a solid support. The single-chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The invention particularly encompasses binding molecules (including antibodies and diabodies) that comprise a VL and/or VH Domain of a humanized antibody. The term “humanized” antibody refers to a chimeric molecule, generally prepared using recombinant techniques, having an epitope-binding site of an immunoglobulin from a non-human species and a remaining immunoglobulin structure of the molecule that is based upon the structure and/or sequence of a human immunoglobulin. The polynucleotide sequence of the variable domains of such antibodies may be used for genetic manipulation to generate such derivatives and to improve the affinity, or other characteristics of such antibodies. It is known that the variable domains of both heavy and light chains contain three complementarity determining regions (CDRs) which vary in response to the antigens in question and determine binding capability, flanked by four framework regions (FRs) which are relatively conserved in a given species and which putatively provide a scaffolding for the CDRs. When non-human antibodies are prepared with respect to a particular antigen, the variable domains can be “reshaped” or “humanized.” The general principle in humanizing an antibody involves retaining the basic sequence of the epitope-binding portion of the antibody, while swapping the non-human remainder of the antibody with human antibody sequences. There are four general steps to humanize a monoclonal antibody. These are: (1) determining the nucleotide and predicted amino acid sequence of the starting antibody light and heavy variable domains (2) designing the humanized antibody or caninized antibody, i.e., deciding which antibody framework region to use during the humanizing or canonizing process (3) the actual humanizing or caninizing methodologies/techniques and (4) the transfection and expression of the humanized antibody. See, for example, U.S. Pat. Nos. 4,816,567; 5,807,715; 5,866,692; and 6,331,415.

A number of humanized antibody molecules comprising an epitope-binding site derived from a non-human immunoglobulin have been described, including chimeric antibodies having rodent or modified rodent Variable Domain and their associated complementarity determining regions (CDRs) fused to human constant domains (see, for example, Lobuglio et al. (1989) “Mouse/Human Chimeric Monoclonal Antibody In Man: Kinetics And Immune Response,” Proc. Natl. Acad. Sci. (U.S.A.) 86:4220-4224 (1989)). Other references describe rodent CDRs grafted into a human supporting framework region (FR) prior to fusion with an appropriate human antibody Constant Domain (see, for example, Riechmann, L. et al. (1988) “Reshaping Human Antibodies for Therapy,” Nature 332:323-327; and Jones et al. (1986) “Replacing The Complementarity-Determining Regions In A Human Antibody With Those From A Mouse,” Nature 321:522-525). Another reference describes rodent CDRs supported by recombinantly veneered rodent framework regions. See, for example, European Patent Publication No. 519,596. These “humanized” molecules are designed to minimize unwanted immunological response towards rodent anti-human antibody molecules, which limits the duration and effectiveness of therapeutic applications of those moieties in human recipients. Other methods of humanizing antibodies that may also be utilized are disclosed by Daugherty et al. (1991) “Polymerase Chain Reaction Facilitates The Cloning, CDR-Grafting, And Rapid Expression Of A Murine Monoclonal Antibody Directed Against The CD18 Component Of Leukocyte Integrins,” Nucl. Acids Res. 19:2471-2476 and in U.S. Pat. Nos. 6,180,377; 6,054,297; 5,997,867; and 5,866,692. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, or six) which differ in sequence relative to the original antibody.

B. Characteristics of Antibody Constant Domains

### 1. Constant Domains of the Light Chain

As indicated above, each Light Chain of an antibody contains a Variable Domain (“VL”) and a Constant Domain (“CL”).

The term “exemplary” as used herein means “non-limiting example(s).” An exemplary CL Domain is a human IgG CL Kappa Domain. The amino acid sequence of an exemplary human CL Kappa Domain is (SEQ ID NO:1):

RTVAAPSVFI FPPSDEQLKS GTASVVCLLN NFYPREAKVQ
WKVDNALQSG NSQESVTEQD SKDSTYSLSS TLTLSKADYE
KHKVYACEVT HQGLSSPVTK SFNRGEC

Alternatively, an exemplary CL Domain is a human IgG CL Lambda Domain. The amino acid sequence of an exemplary human CL Lambda Domain is (SEQ ID NO:2):

QPKAAPSVTL FPPSSEELQA NKATLVCLIS DFYPGAVTVA
WKADSSPVKA GVETTPSKQS NNKYAASSYL SLTPEQWKSH
RSYSCQVTHE GSTVEKTVAP TECS

### 2. Constant Domains of the Heavy Chain

As indicated above, the heavy chains of an antibody may comprise CH1, Hinge Domain, CH2 and CH3 constant domains. The CH1 Domains of the two heavy chains of an antibody complex with the antibody's Light Chain's CL constant region and are attached to the heavy chains CH2 Domains via an intervening Hinge Domain.

An exemplary CH1 Domain is a human IgG1 CH1 Domain. The amino acid sequence of an exemplary human IgG1 CH1 Domain is (SEQ ID NO:3):

ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKRV

An exemplary CH1 Domain is a human IgG4 CH1 Domain. The amino acid sequence of an exemplary human IgG4 CH1 Domain is (SEQ ID NO:4):

ASTKGPSVFP LAPCSRSTSE STAALGCLVK DYFPEPVTVS
WNSGALTSGV HTFPAVLQSS GLYSLSSVVT VPSSSLGTKT
YTCNVDHKPS NTKVDKRV

An exemplary Hinge Domain is a human IgG1 Hinge Domain. The amino acid sequence of an exemplary human IgG1 Hinge Domain is (SEQ ID NO:5):

EPKSCDKTHTCPPCP

Another exemplary Hinge Domain is a human IgG4 Hinge Domain. The amino acid sequence of an exemplary human IgG4 Hinge Domain is (SEQ ID NO:6): ESKYGPPCPSCP. An IgG4 Hinge Domain may comprise a stabilizing mutation such as the S228P substitution. The amino acid sequence of an exemplary S228P-stabilized human IgG4 Hinge Domain is (SEQ ID NO:7): ESKYGPPCPPCP.

The CH2 and CH3 Domains of the two heavy chains of an antibody interact to form an “Fc Domain,” which is a domain that is recognized by cellular Fc Receptors, including but not limited to Fc gamma Receptors (FcγRs). As used herein, the term “Fc Domain” is used to define a C-terminal region of an IgG heavy chain. An Fc Domain is said to be of a particular IgG isotype, class or subclass if its amino acid sequence is most homologous to that isotype relative to other IgG isotypes. In addition to their known uses in diagnostics, antibodies have been shown to be useful as therapeutic agents.

The amino acid sequence of the CH2-CH3 Domain of an exemplary human IgG1 is (SEQ ID NO:8):

```
231 240 250 260 270
APELLGGPSV FLFPPKPKDT LMISRTPEVT CVVVDVSHED
                  280 290 300 310
PEVKFNWYVD GVEVHNAKTK PREEQYNSTY RVVSVLTVLH
                  320 330 340 350
QDWLNGKEYK CKVSNKALPA PIEKTISKAK GQPREPQVYT
                  360 370 380 390
LPPSREEMTK NQVSLTCLVK GEYPSDIAVE WESNGQPENN
                  400 410 420 430
YKTTPPVLDS DGSFFLYSKL TVDKSRWQQG NVFSCSVMHE
                  440 447
ALHNHYTQKS LSLSPGX
```

as numbered by the EU index as set forth in Kabat, wherein X is a lysine (K) or is absent.

The amino acid sequence of the CH2-CH3 Domain of an exemplary human IgG4 is (SEQ ID NO:9):

```
231 240 250 260 270
APEFLGGPSV FLFPPKPKDT LMISRTPEVT CVVVDVSQED
                  280 290 300 310
PEVQFNWYVD GVEVHNAKTK PREEQFNSTY RVVSVLTVLH
                  320 330 340 350
QDWLNGKEYK CKVSNKGLPS SIEKTISKAK GQPREPQVYT
                  360 370 380 390
LPPSQEEMTK NQVSLTCLVK GFYPSDIAVE WESNGQPENN
                  400 410 420 430
YKTTPPVLDS DGSFFLYSRL TVDKSRWQEG NVFSCSVMHE
                  440 447
ALHNHYTQKS LSLSLGX
```

as numbered by the EU index as set forth in Kabat, wherein X is a lysine (K) or is absent.

Throughout the present specification, the numbering of the residues in the constant region of an IgG heavy chain is that of the EU index as in Kabat et al., SEQUENCES OF PROTEINS O_{F} IMMUNOLOGICAL INTEREST, 5^{th} Ed. Public Health Service, NH1, MD (1991), expressly incorporated herein by reference. The term “EU index as in Kabat” refers to the numbering of the constant domains of human IgG1 EU antibody.

Polymorphisms have been observed at a number of different positions within antibody constant regions (e.g., Fc positions, including but not limited to positions 270, 272, 312, 315, 356, and 358 as numbered by the EU index as set forth in Kabat), and thus slight differences between the presented sequence and sequences in the prior art can exist. Polymorphic forms of human immunoglobulins have been well-characterized. At present, 18 Gm allotypes are known: Glm (1, 2, 3, 17) or Glm (a, x, f, z), G2m (23) or G2m (n), G3m (5, 6, 10, 11, 13, 14, 15, 16, 21, 24, 26, 27, 28) or G3m (b1, c3, b3, b0, b3, b4, s, t, g1, c5, u, v, g5) (Lefranc, et al., “The Human IgG Subclasses: Molecular Analysis Of Structure, Function And Regulation.” Pergamon, Oxford, pp. 43-78 (1990); Lefranc, G. et al., 1979, Hum. Genet.: 50, 199-211). It is specifically contemplated that the antibodies of the present invention may incorporate any allotype, isoallotype, or haplotype of any immunoglobulin gene, and are not limited to the allotype, isoallotype or haplotype of the sequences provided herein. Furthermore, in some expression systems the C-terminal amino acid residue (bolded above) of the CH3 Domain may be post-translationally removed. Accordingly, the C-terminal residue of the CH3 Domain is an optional amino acid residue. Specifically encompassed by the instant invention is a B7-H3-ADC lacking the C-terminal residue of the CH3 Domain. Also specifically encompassed by the instant invention are such constructs comprising the C-terminal lysine residue of the CH3 Domain.

The present invention particularly encompasses B7-H3-ADCs comprising anti-B7-H3 Variable Domains (i.e., VL and/or VH Domains) that immunospecifically bind to an epitope of a human B7-H3 polypeptide. Such B7-H3-ADCs are capable of immunospecifically binding to human B7-H3. As used herein such B7-H3 Variable Domains are referred to as “anti-B7-H3-VL” and “anti-B7-H3-VH,” respectively.

II. Anti-B7-113 Antibody mAb-A

An exemplary anti-B7-H3 antibody, designated “mAb-A,” was isolated from hybridoma cells that had been produced through immunization with cells expressing human B7-H3, with a B7-H3 polypeptide or a peptide epitope thereof. Antibody mAb-A was humanized.

Antibody mAb-A was found to be cross-reactive with B7-H3 of cynomolgus monkeys. The amino acid sequences of the VL and VH Domains of mAb-A are provided below. The preferred B7-H3-ADC of the present invention possess all 3 of the CDR_{H}s of the VH Domain, all 3 of the CDR_{L}s of the VL Domain, and optionally the entire VH and VL Domains of humanized monoclonal antibody mAb-A (“hmAb-A”).

### A. Murine Anti-B7-113 Antibody mAb-A

The amino acid sequence of the VL Domain of the murine anti-B7-H3 antibody mAb-A (SEQ ID NO:15) is shown below (CDRL residues are shown underlined):

DIQMTQSPAS LSVSVGETVT ITCRASESIY SYLAWYQQKQ
GKSPQLLVYN TKTLPEGVPS RFSGSGSGTQ FSLKINSLQP
EDFGRYYCQH HYGTPPWTFG GGTNLEIK

The amino acid sequence of the VH Domain of anti-B7-H3 mAb-A (SEQ ID NO:16) is shown below (CDRH residues are shown underlined).

EVQQVESGGD LVKPGGSLKL SCAASGFTFS SYGMSWVRQT
PDKRLEWVAT INSGGSNTYY PDSLKGRFTI SRDNAKNTLY
LQMRSLKSED TAMYYCARHD GGAMDYWGQG TSVTVSS

### B. Humanized Anti-B7-H3 Antibody hmAb-A

The Variable Domains of the anti-B7-H3 antibody mAb-A were humanized to generate a humanized mAb-A (“hmAb-A”). In in some instances alternative humanized Variable Domains were generated to optimize binding activity and/or to remove antigenic epitopes and/or to remove potentially labile amino acid residues.

The amino acid sequence of the VL Domain of hmAb-A (SEQ ID NO:17) is shown below (CDRL residues are shown underlined):

DIQMTQSPSS LSASVGDRVT ITCRASESIY SYLAWYQQKP
GKAPKLLVYN TKTLPEGVPS RFSGSGSGTD FTLTISSLQP
EDFATYYCQH HYGTPPWTFG QGTRLEIK

The amino acid sequence of a Light Chain of hmAb-A comprising the VL of Domain of hmAb-A and a CL Kappa Domain (SEQ ID NO: 19) is shown below:

DIQMTQSPSS LSASVGDRVT ITCRASESIY SYLAWYQQKP
GKAPKLLVYN TKTLPEGVPS RFSGSGSGTD FTLTISSLQP
EDFATYYCQH HYGTPPWTFG QGTRLEIKRT VAAPSVFIFP
PSDEQLKSGT ASVVCLLNNF YPREAKVQWK VDNALQSGNS
QESVTEQDSK DSTYSLSSTL TLSKADYEKH KVYACEVTHQ
GLSSPVTKSF NRGEC

In SEQ ID NO:19, amino acid residues 1-108 correspond to the VL Domain of hmAb-A (SEQ ID NO:17), and amino acid residues 109-215 correspond to the Light Chain kappa constant region (SEQ ID NO:1).

The amino acid sequence of the VH Domain of hmAb-A (SEQ ID NO:18) is shown below (CDR_{H} residues are shown underlined).

EVQLVESGGG LVKPGGSLRL SCAASGFTFS SYGMSWVRQA
PGKGLEWVAT INSGGSNTYY PDSLKGRFTI SRDNAKNSLY
LQMNSLRAED TAVYYCARHD GGAMDYWGQG TTVTVSS

The amino acid sequence of a Heavy Chain comprising the VH Domain of hmAb-A and IgG1 CH1-H-CH2-CH3 Domains (SEQ ID NO:20) is shown below:

EVQLVESGGG LVKPGGSLRL SCAASGFTFS SYGMSWVRQA
PGKGLEWVAT INSGGSNTYY PDSLKGRFTI SRDNAKNSLY
LQMNSLRAED TAVYYCARHD GGAMDYWGQG TTVTVSSAST
KGPSVFPLAP SSKSTSGGTA ALGCLVKDYF PEPVTVSWNS
GALTSGVHTF PAVLQSSGLY SLSSVVTVPS SSLGTQTYIC
NVNHKPSNTK VDKRVEPKSC DKTHTCPPCP APELLGGPSV
FLFPPKPKDT LMISRTPEVT CVVVDVSHED PEVKFNWYVD
GVEVHNAKTK PREEQYNSTY RVVSVLTVLH QDWLNGKEYK
CKVSNKALPA PIEKTISKAK GQPREPQVYT LPPSREEMTK
NQVSLTCLVK GFYPSDIAVE WESNGQPENN YKTTPPVLDS
DGSFFLYSKL TVDKSRWQQG NVFSCSVMHE ALHNHYTQKS
LSLSPGK

In SEQ ID NO:20, amino acids 1-117 correspond to the VH Domain of hmAb-A (SEQ ID NO:18), amino acid residues 118-215 correspond to the IgG1 CH1 Domain (SEQ ID NO:3), amino acid residues 216-230 correspond to the IgG1 Hinge Domain (SEQ ID NO:5), and amino acid residues 231-447 correspond to the IgG1 CH2-CH3 Domain (SEQ ID NO:8). An N-linked glycosylation site is present at Kabat position 296 (shown underlined).

III. Modification of the Fc Domain

The Fc Domain of the Fc Domain-containing molecules (e.g., antibodies and diabodies) of the present invention may be either a complete Fc Domain (e.g., a complete IgG Fc Domain) or only a fragment of an Fc Domain. Optionally, the Fc Domain of the Fc Domain-containing molecules of the present invention lacks the C-terminal lysine amino acid residue.

In traditional immune function, the interaction of antibody-antigen complexes with cells of the immune system results in a wide array of responses, ranging from effector functions such as antibody dependent cytotoxicity, mast cell degranulation, and phagocytosis to immunomodulatory signals such as regulating lymphocyte proliferation and antibody secretion. All of these interactions are initiated through the binding of the Fc Domain of antibodies or immune complexes to specialized cell surface receptors (singularly referred to as an “Fc gamma receptor,” “FcγR,” and collectively as “FcγRs”) found on the surfaces of multiple types of immune system cells (e.g., B lymphocytes, follicular dendritic cells, natural killer cells, macrophages, neutrophils, eosinophils, basophils and mast cells). The diversity of cellular responses triggered by antibodies and immune complexes results from the structural heterogeneity of the three Fc receptors: FcγRI (CD64), FcγRII (CD32), and FcγRIII (CD16). FcγRI (CD64), FcγRIIA (CD32A) and FcγRIII (CD16) are activating (i.e., immune system enhancing) receptors; FcγRIIB (CD32B) is an inhibiting (i.e., immune system dampening) receptor. In addition, interaction with the neonatal Fc Receptor (FcRn) mediates the recycling of IgG molecules from the endosome to the cell surface and release into the blood. The amino acid sequence of exemplary wild-type IgG1 (SEQ ID NO:8) and IgG4 (SEQ ID NO:9) are presented above.

Modification of the Fc Domain may lead to an altered phenotype, for example altered serum half-life, altered stability, altered susceptibility to cellular enzymes or altered effector function. Accordingly, in certain embodiments, the Fc Domain of the Fc Domain-containing molecules of the present invention may be an engineered variant Fc Domain. Although the Fc Domain of the Fc Domain-containing molecules of the present invention may possess the ability to bind to one or more Fc receptors (e.g., FcγR(s)), in particular such variant Fc Domain will have altered binding to FcγRIA (CD64), FcγRIIA (CD32A), FcγRIM (CD32B), FcγRIIIA (CD16a), or FcγRIIIB (CD16b) (relative to the binding exhibited by a wild-type Fc Domain), e.g., will have enhanced binding to an activating receptor and/or will have substantially reduced or no ability to bind to inhibitory receptor(s). Thus, the Fc Domain of the Fc Domain-containing molecules of the present invention may include some or all of the CH2 Domain and/or some or all of the CH3 Domain of a complete Fc Domain, or may comprise a variant CH2 and/or a variant CH3 sequence (that may include, for example, one or more insertions and/or one or more deletions with respect to the CH2 or CH3 domains of a complete Fc Domain). Such Fc Domains may comprise non-Fc polypeptide portions, or may comprise portions of non-naturally complete Fc Domains, or may comprise non-naturally occurring orientations of CH2 and/or CH3 Domains (such as, for example, two CH2 domains or two CH3 domains, or in the N-terminal to C-terminal direction, a CH3 Domain linked to a CH2 Domain, etc.).

In certain embodiments, the Fc Domains of the binding molecules of the present invention exhibit decreased (or substantially no) binding to FcγRIA (CD64), FcγRIIA (CD32A), FcγRIIB (CD32B), FcγRIIIA (CD16a) or FcγRIBB (CD16b) (relative to the binding exhibited by the wild-type IgG1 Fc Domain (SEQ ID NO:8). In certain embodiments, the binding molecules of the present invention comprise an IgG Fc Domain that exhibits reduced ADCC effector function. In a such embodiments, the CH2-CH3 Domains of binding molecules include any 1, 2, 3, or 4 of the substitutions: L234A, L235A, D265A, N297Q, and N297G. In another embodiment, the CH2-CH3 Domains contain an N297Q substitution, an N297G substitution, L234A and L235A substitutions or a D265A substitution, as these mutations abolish FcR binding. Alternatively, a CH2-CH3 Domain of a naturally occurring Fc Domain that inherently exhibits decreased (or substantially no) binding to FcγRIIIA (CD16a) and/or reduced effector function (relative to the binding and effector function exhibited by the wild-type IgG1 Fc Domain (SEQ ID NO:8)) is utilized. In a specific embodiment, the binding molecules of the present invention comprise an IgG4 Fc Domain (SEQ ID:NO:9). When an IgG4 Fc Domain is utilized, the instant invention also encompasses the introduction of a stabilizing mutation, such as the Hinge Domain S228P substitution described herein (see, e.g., SEQ ID NO:7).

The serum half-life of proteins comprising Fc Domains may be increased by increasing the binding affinity of the Fc Domain for FcRn. The term “half-life” as used herein means a pharmacokinetic property of a molecule that is a measure of the mean survival time of the molecules following their administration. Half-life can be expressed as the time required to eliminate fifty percent (50%) of a known quantity of the molecule from a subject's body (e.g., a human patient or other mammal) or a specific compartment thereof, for example, as measured in serum, i.e., circulating half-life, or in other tissues. In general, an increase in half-life results in an increase in mean residence time (MRT) in circulation for the molecule administered. Modifications capable of increasing the half-life of an Fc Domain-containing molecule are known in the art and include, for example M252Y, S254T, T256E, and combinations thereof. For example, see the modifications described in U.S. Pat. Nos. 6,277,375, 7,083,784; 7,217,797, and 8,088,376; U.S. Publication Nos. 2002/0147311; 2007/0148164; and 2011/0081347. .

In one embodiment, a PD-1 X LAG-3 binding molecule of the present invention comprises a variant Fc Region, wherein such variant Fc Region comprises a substitution at position 252 with tyrosine, 254 with threonine, and 256 with glutamate (252Y, 254T and 256E), wherein such numbering is that of the EU index as in Kabat. In a specific embodiment, a PD-1 X LAG-3 binding molecule of the present invention comprises a variant IgG4 Fc Region, wherein such variant IgG4 Fc Region comprises a substitution at position 252 with tyrosine, 254 with threonine, and 256 with glutamate (252Y, 254T and 256E), wherein such numbering is that of the EU index as in Kabat

An exemplary variant IgG4 sequence for the CH2 and CH3 Domains comprising the M252Y/S254T/T256E substitutions is (SEQ ID NO:14):

APEFLGGPSV FLFPPKPKDT LYITREPEVT CVVVDVSQED
PEVQFNWYVD GVEVHNAKTK PREEQFNSTY RVVSVLTVLH
QDWLNGKEYK CKVSNKGLPS SIEKTISKAK GQPREPQVYT
LPPSQEEMTK NQVSLTCLVK GFYPSDIAVE WESNGQPENN
YKTTPPVLDS DGSFFLYSRL TVDKSRWQEG NVFSCSVMHE
ALHNHYTQKS LSLSLGX

wherein, X is a lysine (K) or is absent.

### IV. B7-H3-ADC

The present invention relates to the above-described anti-B7-H3 antibody hmAb-A conjugated to a cytotoxic drug, a B7-H3-ADC. Such B7-H3-ADC enhances the cytotoxicity of anti-B7-H3 therapy, particularly in the treatment of cancer. As indicated above, a B7-H3-ADC of the present invention are represented by the formula:

Ab-(LM)ₘ-(D)ₙ,

wherein:
Ab is an antibody that binds to B7-H3 that comprises a humanized Variable Heavy Chain (VH) Domain and a humanized Variable Light Chain (VL) Domain, or is a B7-H3-binding fragment thereof, and;
D is a cytotoxic duocarmycin moiety;
LM is a bond or a Linker Molecule that covalently links Ab and D;
m is an integer between 0 and n and denotes the number of bonds or Linker Molecules of the B7-H3-ADC, except when LM is a bond, m is not 0;
and
n is an integer between 1 and 10 and denotes the number of cytotoxic duocarmycin moieties covalently linked to the B7-H3-ADC.

In certain embodiments, a B7-H3-ADC of the present invention comprises a naturally occurring Fc Domain of the IgG1 isotype. Such Fc Domain lacks the C-terminal lysine residue of a CH3 Domain. In specific embodiments, a B7-H3-ADC will bind to a tumor cell expressing B7-H3 and will then be internalized into such cell through receptor-mediated endocytosis. Once inside a lysosome, a B7-H3-ADC will preferably be degraded so as to thereby cause the release of the cytotoxic duocarmycin moiety inside the cell, resulting in cell death. As will be appreciated, the mechanism of action of cell death can vary based on the class of cytotoxic drug used (e.g., disruption of cytokinesis by tubulin polymerization inhibitors such as maytansines and auristatins, DNA damage by DNA interacting agents such as calicheamicins and duocarmycins), etc. Neighboring cancer cells may also be killed when free drug is released into the tumor environment by the dying cell in a process known as the bystander effect (Panowski, S. et al. (2014) “Site-Specific Antibody Drug Conjugates For Cancer Therapy,” mAbs 6(1):34-45; Kovtun, Y. V. et al. (2006) “Antibody-Drug Conjugates Designed To Eradicate Tumors With Homogeneous And Heterogeneous Expression Of The Target Antigen,” Cancer Res. 66:3214-3221).

A. Exemplary Linker Molecules of the Invention

The invention particularly contemplates such B7-H3-ADCs wherein LM is a Linker Molecule and is absent (i.e., m=0), and B7-H3-ADCs that possess more than one Linker Molecule LM (i.e., m is an integer from 2 through n, wherein n is an integer from 2 through 10), each of which Linker Molecule LM covalently links a cytotoxic duocarmycin moiety D to the Ab of such B7-H3-ADCs.

The invention further provides B7-H3-ADCs whose Ab are covalently linked to more than one Linker Molecule LM, wherein all such Linker Molecules are identical. The cytotoxic duocarmycin moieties D that are covalently linked to the Ab of such B7-H3-ADCs may all be identical or may include 2, 3, 4, or more independently different cytotoxic duocarmycin moieties D.

The invention further provides such B7-H3-ADCs whose Ab are covalently linked to more than one Linker Molecule LM, wherein all such Linker Molecules are not identical and may independently differ. The cytotoxic duocarmycin moieties D that are covalently linked to the Ab of such B7-H3-ADCs may all be identical or may include 2, 3, 4, or more independently different cytotoxic duocarmycin moieties D.

Exemplary humanized VH and VL Domains of antibodies that bind to human B7-H3, and exemplary human antibody Constant Domains that may be included in a B7-H3-ADC are provided above. As stated above, a B7-H3-ADC additionally comprise at least one cytotoxic duocarmycin moiety, which is covalently linked to an atom of a side chain of an amino acid residue of such VH Domain or VL Domain and/or Constant Domain, either directly, or via a Linker Molecule intercalated between the side chain atom and the duocarmycin moiety. The Linker Molecule may be a non-peptide molecule, or a molecule that comprises a non-peptide portion and a peptide portion, or it may be a molecule that is composed solely of amino acid residues. The amino acid residues of any such Linker Molecules may contain naturally occurring or non-naturally occurring amino acid residues, including D-versions of naturally occurring amino acid residues, p-acetylphenylalanine, selenocysteine, etc. Optionally, or additionally, particular residues having a desired side chain (e.g., a —CH₂—SH side chain, a —CH₂—OH side chain, a —CH(CH₂)—SH side chain, a —CH₂—CH₂—S—CH₃ side chain; a —CH₂—C(O)—NH₂ side chain, a —CH₂—CH₂—C(O)—NH₂ side chain, a —CH₂—C(O)OH— side chain, a CH₂—CH₂—C(O)OH— side chain, a —CH₂—CH₂—CH₂—CH₂—NH₂ side chain, a —CH₂—CH₂—CH₂—NH—C(NH₂)₂ side chain, an imidazole side chain, a benzyl side chain, a phenol side chain, an indole side chain, etc.) may be engineered into a B7-H3-ADC.

The Linker Molecule LM may be non-cleavable under physiologic conditions, for example composed of a hydrolytically stable moiety, for example, a thioether linker or a hindered disulfide linker. Hydrolytically stable linkers are substantially stable in water and do not react with water at useful pH values, including but not limited to, under physiological conditions for an extended period of time. In contrast, hydrolytically unstable or degradable linkers are degradable in water or in aqueous solutions, including for example, blood.

Alternatively, the Linker Molecule LM may be cleavable, or may contain a cleavable portion. Examples of such a cleavable portion includes an acid labile linker (e.g., a 4-(4′-acetylpheonxy)butanoic acid linker which forms a hydrazine bond), a cleavable disulfide linker (that is cleaved in the reducing intracellular environment), and a protease cleavable linker. Acid-labile linkers are designed to be stable at pH levels encountered in the blood, but become unstable and degrade when the low pH environment in lysosomes is encountered. Protease-cleavable linkers are also designed to be stable in blood/plasma, but rapidly release free drug inside lysosomes in cancer cells upon cleavage by lysosomal enzymes (Panowski, S. et al. (2014) “Site-Specific Antibody Drug Conjugates For Cancer Therapy,” mAbs 6(1):34-45). Alternatively, the Linker Molecule may be an enzyme-cleavable-substrate or contain an enzyme-cleavable-substrate, such as a cleavable peptide, (e.g., a cleavable dipeptide such as a valine-citrulline dipeptide para-aminobenzylalcohol linker (cAC10-mc-vc-PABA) which is selectively cleaved by lysosomal enzymes). Suitable cleavable linkers are known in the art, see, e.g., de Groot, Franciscus M. H., et al. (2002) “Design, Synthesis, and Biological Evaluation of a Dual Tumor-Specific Motive Containing Integrin-Targeted Plasmin-Cleavable Doxorubicin Prodrug,” Molecular Cancer Therapeutics, 1: 901-911; Dubowchik et al., (2002) “Doxorubicin Immunoconjugates Containing Bivalent, Lysosomally-Cleavable Dipeptide Linkages.” Bioorganic & Medicinal Chemistry Letters12:1529-1532; U.S. Pat. Nos. 5547667; 6,214,345; 7,585,491; 7,754,681; 8,080,250; 8,461,117; and WO 02/083180.

Enzymatically unstable or degradable linkers can be employed. Such linkers are degraded by one or more enzymes. By way of example only, PEG and related polymers can include a degradable Linker Molecule(s) in the polymer backbone or in the linker group between the polymer backbone and one or more of the terminal functional groups of the polymer molecule. Such degradable Linker Molecule(s) include, but are not limited to, ester linkages formed by the reaction of PEG carboxylic acids or activated PEG carboxylic acids with alcohol groups on a biologically active agent, wherein such ester groups generally hydrolyze under physiological conditions to release the biologically active agent. Other hydrolytically degradable Linker Molecules include but are not limited to carbonate linkages; imine linkages resulting from reaction of an amine and an aldehyde; phosphate ester linkages formed by reacting an alcohol with a phosphate group; hydrazone linkages that are a reaction product of a hydrazide and an aldehyde; acetal linkages that are the reaction product of an aldehyde and an alcohol; orthoester linkages that are the reaction product of a formate and an alcohol; peptide linkages formed by an amine group, including but not limited to, at an end of a polymer such as PEG, and a carboxyl group of a peptide; and oligonucleotide linkages formed by a phosphoramidite group, including but not limited to, at the end of a polymer, and a 5′ hydroxyl group of an oligonucleotide.

In one embodiment, the Linker Molecule of the present invention may be, or may comprise, a cleavable Linker Molecule, V-(W)ₖ-(X)₁-A, as disclosed in PCT Publication WO 02/083180, resulting in a B7-H3-ADC having the formula:

Ab-[V-(W)ₖ-(X)₁-A]-D

wherein:
V is an optional cleavable moiety,
(W)ₖ-(X)₁-A is an elongated, self-eliminating spacer system, that self-eliminates via a l, (4+2n)-elimination,
W and X are each a l, (4+2n) electronic cascade spacer, being the same or different,
A is either a spacer group of formula (Y)ₘ, wherein Y is a l, (4+2n) electronic cascade spacer, or a group of formula U, being a cyclisation elimination spacer,
k, l and m are independently an integer of 0 (included) to 5 (included),
n is an integer of 0 (included) to 10 (included),
   with the provisos that:
when A is (Y)ₘ: then k+l+m≥1, and
if k+l+m=1, then n>l;
when A is U: then k+l≥1.
   W, X, and Y are independently selected from compounds having the formula:

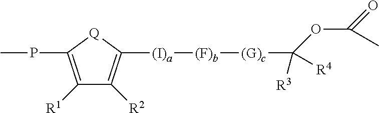

or the formula:

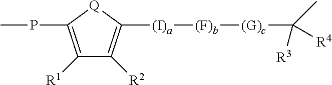

wherein: Q is —R⁵C═CR⁶—, S, O, NR^{S}, —R⁵C═N—, or —N═CR⁵—
   P is NR⁷, O or S
   a, b, and c are independently an integer of 0 (included) to 5(included);
   I, F and G are independently selected from compounds having the formula:

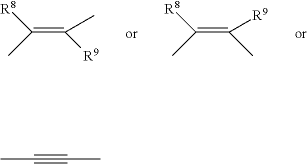

wherein R¹, R², R³, R⁴, R⁵, R⁶, R⁷, R⁸, and R⁹ independently represent H, C₁₋₆ alkyl, C₃₋₂₀ heterocyclyl, C₅₋₂₀ aryl, C₁₋₆ alkoxy, hydroxy (OH), amino (NH₂), mono-substituted amino (NRₓH), di-substituted amino (NRₓ¹Rₓ²), nitro (NO₂), halogen, CF₃, CN, CONH₂, SO₂Me, CONHMe, cyclic C₁₋₅ alkylamino, imidazolyl, C₁₋₆ alkylpiperazinyl, morpholino, thiol (SH), thioether (SRₓ), tetrazole, carboxy (COOH), carboxylate (COORₓ), sulphoxy (S(═O)₂OH), sulphonate (S(═O)₂ORₓ), sulphonyl (S(═O)₂Rₓ), sulphixy (S(═O)OH), sulphinate (S(═O)ORₓ), sulphinyl (S(═O)Rₓ), phosphonooxy (OP(═O)(OH)₂), and phosphate (OP(═O)(ORₓ)₂), where Rₓ, Rₓ¹ and Rₓ² are independently selected from a C₁₋₆ alkyl group, a C₃₋₂₀ heterocyclyl group or a C₅₋₂₀ aryl group, two or more of the substituents R¹, R², R³, R⁴, R⁵, R⁶, R⁷, R⁸, or R⁹ optionally being connected to one another to form one or more aliphatic or aromatic cyclic structures;
   U is selected from compounds having the formula:

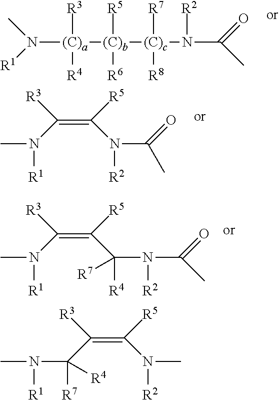

wherein:
   a, b and c are independently selected to be an integer of 0 or 1;
      provided that a+b+c=2 or 3;
   R¹ and/or R² independently represent H, C1-6 alkyl, said alkyl being optionally substituted with one or more of the following groups: hydroxy (OH), ether (ORₓ), amino (NH₂), mono-substituted amino (NRₓH), disubstituted amino (NRₓ¹Rₓ²), nitro (NO₂), halogen, CF₃, CN, CONH₂, SO₂Me, CONHMe, cyclic C₁₋₅ alkylamino, imidazolyl, C₁₋₆ alkylpiperazinyl, morpholino, thiol (SH), thioether (SR_{X}), tetrazole, carboxy (COOH), carboxylate (COORₓ), sulphoxy (S(═O)₂OH), sulphonate (S(═O)₂OR_{X}), sulphonyl (S(═O)₂Rₓ), sulphixy (S(═O)OH), sulphinate (S(═O)ORx), sulphinyl (S(═O)Rx), phosphonooxy (OP(═O)(OH)₂), and phosphate (OP(═O)(ORₓ)₂), where Rₓ, Rₓ¹ and Rₓ² are selected from a C₁₋₆ alkyl group, a C₃₋₂₀ heterocyclyl group or a C₅₋₂₀ aryl group; and
   R³, R⁴, R⁵, R⁶, R⁷ and R⁸ independently represent H, C₁₋₆ alkyl, C₃₋₂₀ heterocyclyl, C₅₋₂₀ aryl, C₁₋₆ alkoxy, hydroxy (OH), amino (NH₂), mono-sub stituted amino (NRₓH), disubstituted amino (NRₓ¹Rₓ²), nitro (NO₂), halogen, CF₃, CN, CONH₂, SO₂Me, CONHMe, cyclic C₁₋₅ alkylamino, imidazolyl, C₁₋₆ alkylpiperazinyl, morpholino, thiol (SH), thioether (SRₓ), tetrazole, carboxy (COOH), carboxylate (COORₓ), sulphoxy (S(═O)₂OH), sulphonate (S(═O)₂ORₓ), sulphonyl (S(═O)₂Rₓ), sulphixy (S(═O)OH), sulphinate (S(═O)ORₓ), sulphinyl (S(═O)Rₓ), phosphonooxy (OP(═O)(OH)₂), and phosphate (OP(═O)(ORₓ)₂), where Rₓ, Rₓ¹ and Rₓ² are selected from a C₁₋₆ alkyl group, a C₃₋₂₀ heterocyclyl group or a C₅₋₂₀ aryl group, and two or more of the substituents R², R³, R⁴, R⁵, R⁶, R⁷, or R⁸ are optionally connected to one another to form one or more aliphatic or aromatic cyclic structures.

Exemplary molecules include:
p-aminobenzyloxycarbonyl-p-aminobenzyloxycarbonyl;
p-aminobenzyloxycarbonyl-p-aminobenzyloxycarbonyl-p-aminobenzyloxycarbonyl;
p-ammocinnamyloxycarbonyl;
p-aminocinnamyloxycarbonyl-p-aminobenzyloxycarbonyl;
p-amino-benzyloxycarbonyl-p-aminocinnamyloxycarbonyl;
p-aminocinnamyloxycarbonyl-p-aminocinnamyloxycarbonyl;
p-aminophenylpentadienyloxycarbonyl;
p-aminophenylpentadienyloxycarbonyl-p-aminocinnamyloxycarbonyl;
p-aminophenylpentadienyloxycarbonyl-paminobenzyloxycarbonyl;
p-aminophenylpentadienyloxycarbonyl-p-aminophenylpentadienyloxycarbonyl;
p-aminobenzyloxycarbonyl(methylamino)ethyl(methylamino)carbonyl;
p-aminocinnamyloxycarbonyl(methylamino)ethyl(methylamino)carbonyl;
p-aminobenzyloxycarbonyl-p-aminobenzyloxycarbonyl(methylamino)ethyl(methylamino)carbonyl;
p-aminocinnamyloxycarbonyl-p-aminobenzyloxycarbonyl(methylamino)ethyl(methylamino)carbonyl;
p-aminobenzyloxycarbonyl-p-aminocinnamyloxycarbonyl(methylamino)ethyl(methylamino)-carbonyl;
p-aminocinnamyloxycarbonyl-p-aminocinnamyloxycarbonyl(methylamino)ethyl(methylamino)carbonyl;
p-aminobenzyloxycarbonyl-p-aminobenzyl;
p-aminobenzyloxycarbonyl-p-aminobenzyloxycarbonyl-p-aminobenzyl;
p-aminocinnamyl;
p-aminocinnamyloxycarbonyl-p-aminobenzyl;
p-aminobenzyloxycarbonyl-p-aminocinnamyl;
p-amino-cinnamyloxycarbonyl-p-aminocinnamyl;
p-aminophenylpentadienyl;
p-aminophenylpentadienyloxycarbonyl-p-aminocinnamyl;
p-aminophenylpentadienyloxycarbonyl-p-aminobenzyl;
   and
p-aminophenylpentadienyloxycarbonyl-p-aminophenylpentadienyl.

In some embodiments, a B7-H3-ADC comprises two, three, four, five, six, seven, eight, nine or ten cytotoxic duocarmycin moieties, which may be the same, or may independently be the same or different from another cytotoxic duocarmycin moiety of the B7-H3-ADC . In one embodiment, each such cytotoxic duocarmycin moiety is conjugated to the Ab of a B7-H3-ADC via a separate Linker Molecule. Alternatively, more than one cytotoxic duocarmycin moiety may be attached to the Ab of a B7-H3-ADC via the same Linker Molecule.

Cytotoxic duocarmycin moieties may be conjugated to the Ab of a B7-H3-ADC by means known in the art (see, e.g., Yao, H. et al. (2016) “Methods to Design and Synthesize Antibody-Drug Conjugates (ADC),” Intl. J. Molec. Sci. 17(194):1-16); Behrens, C. R. et al. (2014) “Methods For Site-Specific Drug Conjugation To Antibodies,” mAbs 6(1):46-53; Bouchard, H. et al. (2014) “Antibody-Drug Conjugates—A New Wave Of Cancer Drugs,” Bioorganic & Medicinal Chem. Lett 24:5357-5363). The thiol group of a cysteine, the amino side group of lysine, glutamine or arginine, or the carboxyl group of glutamate or aspartate can be employed to conjugate the Linker Molecule-cytotoxic duocarmycin moiety (LM-D) to the Ab of a B7-H3-ADC . Native antibodies contain numerous lysine conjugation sites, and thus are capable of linking multiple conjugated molecules per antibody. Indeed, peptide mapping has determined that conjugation occurs on both the heavy and light chain at approximately 20 different lysine residues (40 lysines per mAb). Therefore, greater than one million different ADC species can be generated. Cysteine conjugation occurs after reduction of one to four inter-chain disulfide bonds, and the conjugation is thus limited in native VL and VH Domains to the eight exposed sulfhydryl groups. However, if desired, additional reactive (e.g., lysine, cysteine, selenocysteine, etc.) residues may be engineered into an antibody (e.g., within a VL Domain and/or a VH Domain and/or a Constant Domain). For example, one or more native amino acid residues may be substituted with a cysteine residue. An unnatural amino acid (e.g. p-acetylphenylalanine) may be genetically incorporated into an antibody using an amber stop codon suppressor tRNA/aaRS pair. (See, e.g., Behrens C R, and Liu B. (2014) “Methods For Site-Specific Drug Conjugation To Antibodies,” mAbs 6(1):46-53. doi:10.4161/mabs.26632; Panowksi, S., et al. (2014) “Site-Specific Antibody Drug Conjugates For Cancer Therapy,” mAbs, 6(1), 34-45, doi:10.4161/mabs.27022; and WO 2008/070593). Alternatively, or additionally, enzymes (e.g., a glycotransferase) may be used to conjugate the Linker Molecule-cytotoxic duocarmycin moiety (LM-D) to the Ab of a B7-H3-ADC. The glycotransferase platform attaches a sugar moiety to a glycosylation site on an antibody (for example, position N297 of the Fc Domain of a human IgG antibody), which can then serve as the Linker Molecule of the present invention and conjugate the cytotoxic duocarmycin moiety (D) to the Ab of a B7-H3-ADC. Alternatively, a transglutaminase may be used to catalyze the formation of a covalent bond between a free amine group and a glutamine side chain.

An exemplary transglutaminase is the commercially available transglutaminase from Streptoverticillium mobaraense (mTG) (Pasternack, R. et al. (1998) “Bacterial Pro-Transglutaminase From Streptoverticillium mobaraense—Purification, Characterisation And Sequence Of The Zymogen,” Eur. J. Biochem. 257(3):570-576; Yokoyama, K. et al. (2004) “Properties And Applications Of Microbial Transglutaminase,” Appl. Microbiol. Biotechnol. 64:447-454). This enzyme does not recognize any of the natural occurring glutamine residues in the Fc Domain of glycosylated antibodies, but does recognize the tetrapeptide LLQL (SEQ ID NO:21) (Jeger, S. et al. (2010) “Site-Specific And Stoichiometric Modification Of Antibodies By Bacterial Transglutaminase,” Angew Chem. Int. Ed. Engl. 49:9995-9997) that may be engineered into a VL Domain and/or a VH Domain and/or a Constant Domain. Such considerations are reviewed by Panowski, S. et al. (2014) “Site-Specific Antibody Drug Conjugates For Cancer Therapy,” mAbs 6(1):34-45.

### B. Exemplary Duocarmycin Moieties of the Invention

Duocarmycins are members of a series of related natural products first isolated from Streptomyces bacteria and they are potent antitumor antibiotics (see Dokter, W. et al. (2014) “Preclinical Profile of the HER2-Targeting ADC SYD983/SYD985: Introduction of a New Duocarmycin-Based Linker-Drug Platform,” Mol. Cancer Ther. 13 (11) :2618-2629; Boger, D. L. et al. (1991). “Duocarmycins—A New Class Of Sequence Selective DNA Minor Groove Alkylating Agents,” Chemtracts: Organic Chemistry 4 (5): 329-349 (1991); Tercel et al. (2013) “The Cytotoxicity Of Duocarmycin Analogues Is Mediated Through Alkylation Of DNA, Not Aldehyde Dehydrogenase 1: A Comment,” Chem. Int. Ed. Engl. 52(21):5442-5446; Boger, D. L. et al. (1995) “CC-1065 And The Duocarmycins: Unraveling The Keys To A New Class Of Naturally Derived DNA Alkylating Agents,” Proc. Natl. Acad. Sci. (U.S.A.) 92(9):3642-3649; Cacciari, B. et al. (2000) “CC-1065 And The Duocarmycins: Recent Developments,” Expert Opinion on Therapeutic Patents 10(12):1853-1871).

Natural duocarmycins include duocarmycin A, duocarmycin B1, doucarmycin B2, duocarmycin C1, duocarmycin C2, duocarmycin D, duocarmycin SA, and CC-1065 (PCT Publication No. WO 2010/062171; Martin, D. G. et al. (1980) “Structure Of CC-1065 (NSC 298223), A New Antitumor Antibiotic,” J. Antibiotics 33:902-903; Boger, D. L. et al. (1995) “CC-1065 And The Duocarmycins: Unraveling The Keys To A New Class Of Naturally Derived DNA Alkylating Agents,” Proc. Natl. Acad. Sci. (U.S.A.) 92:3642-3649).

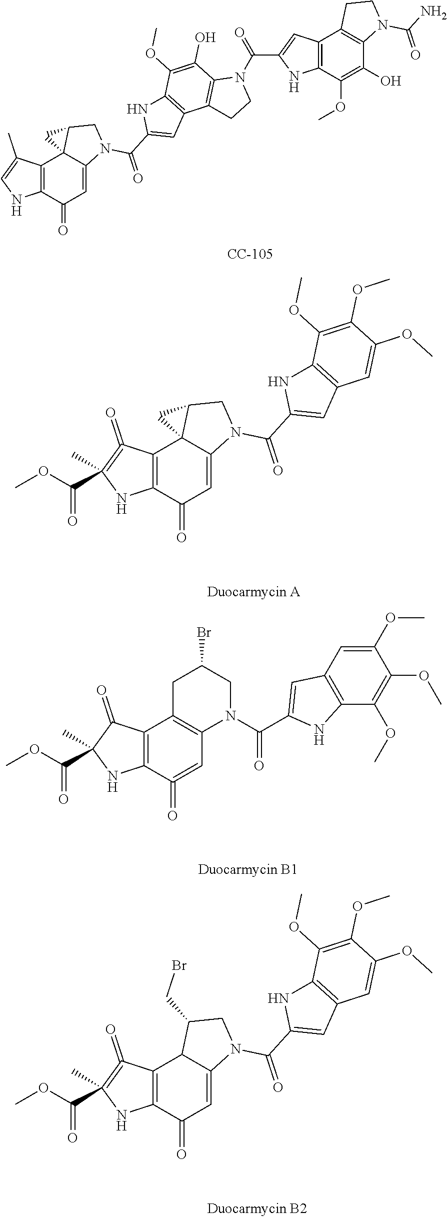

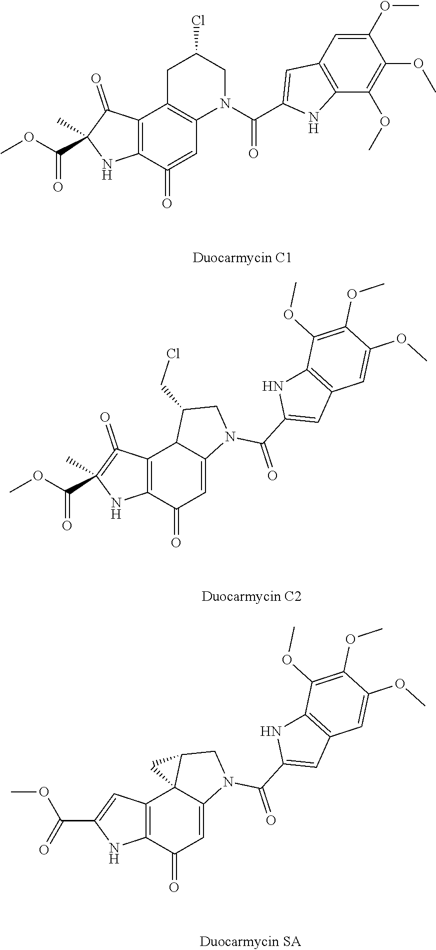

Suitable synthetic duocarmycin analogs include adozelesin, bizelesin, carzelesin (U-80244) and spiro-duocarmycin (DUBA) (Dokter, W. et al. (2014) “Preclinical Profile of the HER2-Targeting ADC SYD983/SYD985: Introduction of a New Duocarmycin-Based Linker-Drug Platform,” Mol. Cancer Ther. 13(11):2618-2629; Elgersma, R. C. et al. (2014) “Design, Synthesis, and Evaluation of Linker-Duocarmycin Payloads: Toward Selection of HER2-Targeting Antibody-Drug Conjugate SYD985,” Mol. Pharmaceut. 12:1813-1835):

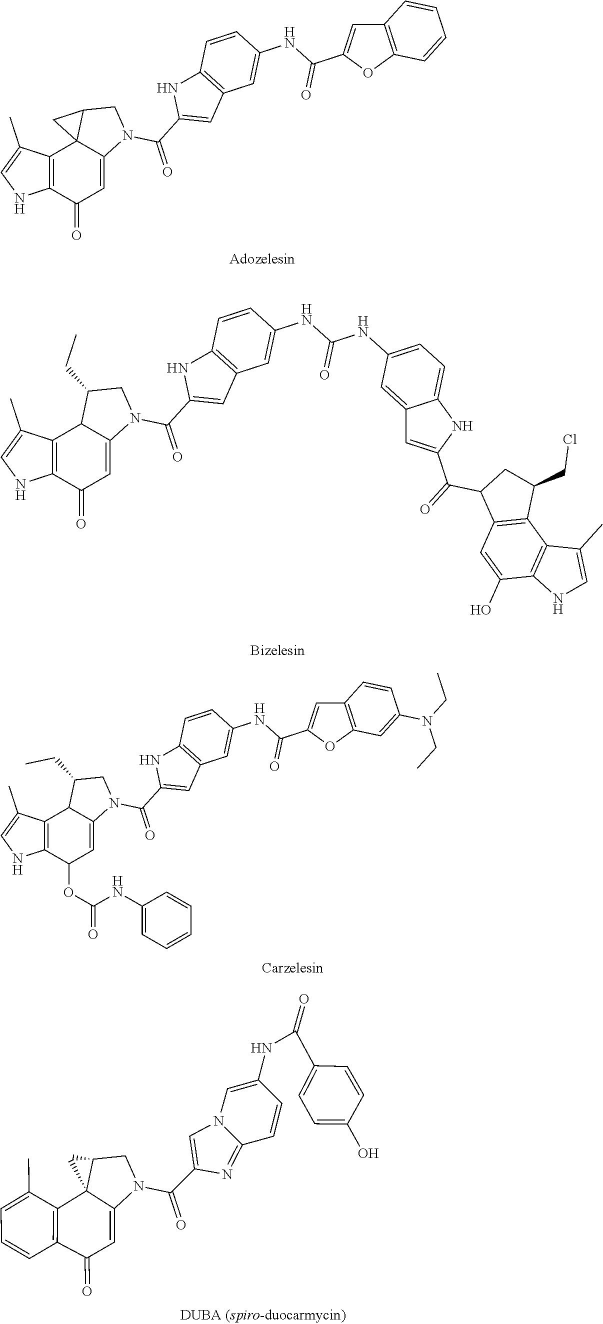 [SMILES: Cc1c[nH]c2c1C13CC1CN(C(=O)c1cc4cc(NC(=O)c5cc6ccccc6o5)ccc4[nH]1)C3=CC2=O ; CC[C@@H]1CN(C(=O)c2cc3cc(NC(=O)Nc4ccc5[nH]c(C(=O)C6C[C@@H](CCl)c7c6cc(O)c6[nH]cc(C)c76)cc5c4)ccc3[nH]2)C2=CC(=O)c3[nH]cc(C)c3C21 ; CC[C@@H]1CN(C(=O)c2cc3cc(NC(=O)c4cc5ccc(N(CC)CC)cc5o4)ccc3[nH]2)C2=CC(OC(=O)Nc3ccccc3)c3[nH]cc(C)c3C21 ; Cc1cccc2c1[C@@]13C[C@@H]1CN(C(=O)c1cn4cc(NC(=O)c5ccc(O)cc5)ccc4n1)C3=CC2=O]

Additional synthetic duocarmycin analogs include those disclosed in PCT Publication No. WO 2010/062171, and particularly such analogs that have the formula:

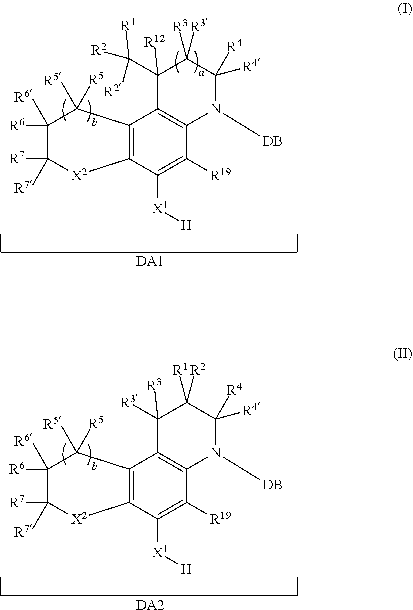

or a pharmaceutically acceptable salt, hydrate, or solvate thereof, wherein DB is a DNA-binding moiety and is selected from the group consisting of:

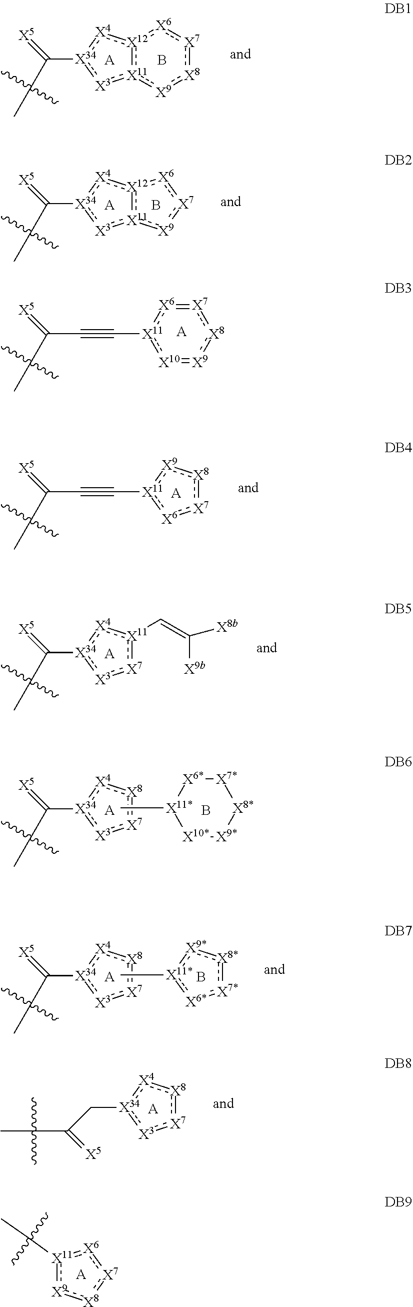

wherein:
R is a leaving group;
R², R^{2′}, R³, R^{3′}, R⁴, R^{4′}, R¹², and R¹⁹ are independently selected from H, OH, SH, NH₂, N₃, NO₂, NO, CF₃, CN, C(O)NH2, C(O)H, C(O)OH, halogen, Ra, SR^{a}, S(O)R^{a}, S(O)2R^{a}, S(O)OR^{a}, S(O)₂OR^{a}, OS(O)R^{a}, OS(O)₂R^{a}, OS(O)OR^{a}, OS(O)₂OR^{a}, OR^{a}, NHR^{a}, N(R^{a})R^{b}, +N(R^{a})(R^{b})R^{c}, P(O)(OR^{a})(OR^{b}), OP(O)(OR^{a})(OR^{b}), SiR^{a}R^{b}R^{c}, C(O)R^{a}, C(O)OR^{a}, C(O)N(R^{a})R^{b}, OC(O)R^{a}, OC(O)OR^{a}, OC(O)N(R^{a})R^{b}, N(R^{a})C(O)R^{b}, N(R^{a})C(O)OR^{b}, and N(R^{a})C(O)N(R^{b})R^{c}, wherein R^{a}, R^{b}, and R^{c} are independently selected from H and optionally substituted C₁₋₃ alkyl or C₁₋₃ heteroalkyl, or R³+R^{3′} and/or R⁴+R^{4′} are independently selected from ═O, ═S, ═NOR¹⁸, ═C(R¹⁸)R^{18′}, and ═NR¹⁸, R¹⁸ and R^{18′} being independently selected from H and optionally substituted C₁₋₃ alkyl, two or more of R², R^{2′}, R³, R^{3′}, R⁴, R^{4′} and R¹² optionally being joined by one or more bonds to form one or more optionally substituted carbocycles and/or heterocycles;
X² is selected from O, C(R¹⁴)(R^{14′}), and NR^{14′}, wherein R¹⁴ and R^{14′} have the same meaning as defined for R⁷ and are independently selected, or R^{14′} and R^{7′} are absent resulting in a double bond between the atoms designated to bear R^{7′} and R^{14′};
R⁵, R^{5′}, R⁶, R^{6′}, R⁷, and R^{7′} are independently selected from H, OH, SH, NH₂, N₃, NO₂, NO, CF₃, CN, C(O)NH₂, C(O)H, C(O)OH, halogen, R^{e}, SR^{e}, S(O)R^{e}, S(O)₂R^{e}, S(O)OR^{e}, S(O)₂OR^{e}, OS(O)R^{e}, OS(O)₂R^{e}, OS(O)OR^{e}, OS(O)₂OR^{e}, OR^{e}, NHR^{e}, N(R^{e})R^{f}, ⁺N(R^{e})(R^{f})R^{g}, P(O)(OR^{e})(OR^{f}), OP(O)(OR^{e})(OR^{f}), SiR^{e}R^{f}R^{g}, C(O)R^{e}, C(O)OR^{e}, C(O)N(R^{e})R^{f}, OC(O)R^{e}, OC(O)OR^{e}, OC(O)N(R^{e})R^{f}, N(R^{e})C(O)R^{f}, N(R^{e})C(O)OR^{f}, N(R^{e})C(O)N(R^{f})R^{g}, and a water-soluble group,
wherein
R^{e}, R^{f}, and R^{g} are independently selected from H and optionally substituted (CH₂CH₂O)ₑₑCH₂CH₂X¹³R^{e1}, C₁₋₁₅ alkyl, C₁₋₁₅ heteroalkyl, C₃₋₁₅ cycloalkyl, C₁₋₁₅ heterocycloalkyl, C₅₋₁₅ aryl, or C₁₋₁₅ heteroaryl, wherein ee is selected from 1 to 1000, X¹³ is selected from O, S, and NR^{f1}, and R^{f1} and R^{e1} are independently selected from H and C₁₋₃ alkyl, one or more of the optional substituents in R^{e}, R^{f}, and/or R^{g} optionally being a water-soluble group, two or more of R^{e}, R^{f}, and R^{g} optionally being joined by one or more bonds to form one or more optionally substituted carbocycles and/or heterocycles, or R⁵+R^{5′} and/or R⁶+R^{6′} and/or R⁷+R^{7′} are independently selected from ═O, ═S, ═NOR^{e3}, ═C(R^{e3})R^{e4}, and ═NR^{e3}, R^{e3} and R^{e4} being independently selected from H and optionally substituted C₁₋₃ alkyl, or R^{5′}+R^{6′} and/or R^{6′}+R^{7′} and/or R^{7′}+R^{14′} are absent, resulting in a double bond between the atoms designated to bear R^{5′}+R^{6′} and/or R^{6′}+R^{7′} and/or R^{7′}+R^{14′} respectively, two or more of R⁵, R^{5′}, R⁶, R^{6′}, R⁷, R^{7′}, R¹⁴ and R^{14′} optionally being joined by one or more bonds to form one or more optionally substituted carbocycles and/or heterocycles;
X¹ is selected from O, S, and NR, wherein R is selected from H and optionally substituted C₁₋₈ alkyl or C₁₋₈ heteroalkyl and not joined with any other substituent;
X³ is selected from O, S, C(R¹⁵)R^{15′}, —C(R¹⁵)(R^{15′})—C(R^{15″})(R^{15′″})—, —N(R¹⁵)—N(R^{15′})—, —C(R¹⁵)(R^{15′})—N(R^{15″})—, —N(R^{15″})—C(R¹⁵)(R^{15′})—, —C(R¹⁵)(R^{15′})—O—, —O—C(R¹⁵)(R^{15′})—, —C(R¹⁵)(R^{15′})—S—, —S—C(R¹⁵)(R^{15′})—, —C(R¹⁵)═C(R^{15′})—, ═C(R¹⁵)—C(R^{15′})═, —N═C(R^{15′})—, ═N—C(R^{15′})═, —C(R¹⁵)═N—, ═C(R¹⁵)—N═, —N═N—, ═N—N═, CR¹⁵, N, NR¹⁵, or in DB1 and DB2-X3- represents —X^{3a} and X^{3b}—, wherein X^{3a} is connected to X³⁴, a double bond is present between X³⁴ and X⁴, and X^{3b} is connected to X¹¹, wherein X^{3a} is independently selected from H and optionally substituted (CH₂CH₂O)ₑₑCH₂CH₂X¹³R^{e1}, C₁₋₈ alkyl, or C₁₋₈ heteroalkyl and not joined with any other substituent;
X⁴ is selected from O, S, C(R¹⁶)R^{16′}, NR¹⁶, N, and CR¹⁶;
X⁵ is selected from O, S, C(R¹⁷)R^{17′}, NOR¹⁷, and NR¹⁷, wherein R¹⁷ and R^{17′} are independently selected from H and optionally substituted C₁₋₈ alkyl or C₁₋₈ heteroalkyl and not joined with any other substituent;
X⁶ is selected from CR¹¹, CR¹¹(R^{11′}), N, NR¹¹, O, and S;
X⁷ is selected from CR⁸, CR⁸(R^{8′}), N, NR⁸, O, and S;
X⁸ is selected from CR⁹, CR⁹(R⁹′), N, NR⁹, O, and S;
X⁹ is selected from CR¹⁰, CR¹⁰(R^{10′}), N, NR¹⁰, O, and S;
X¹⁰ is selected from CR²⁰, CR²⁰(R^{20′}), N, NR²⁰, O, and S;
X¹¹ is selected from C, CR²¹, and N, or X¹¹-X^{3b} is selected from CR²¹, CR²¹(R^{21′}) N, NR²¹, O, and S;
X¹² is selected from C, CR²², and N;
X⁶*, X⁷*, X⁸*, X⁹*, X¹⁰*, and X¹¹* have the same meaning as defined for X⁶, X⁷, X⁸, X⁹, X¹⁰, and X¹¹, respectively, and are independently selected;
X³⁴ is selected from C, CR²³, and N;
the ring B atom of X¹¹* in DB6 and DB7 is connected to a ring atom of ring A such that ring A and ring B in DB6 and DB7 are directly connected via a single bond;
a dashed double bond means that the indicated bond may be a single bond or a non-cumulated, optionally delocalized, double bond;
R⁸, R^{8′}, R⁹, R^{9′}, R¹⁰, R^{10′}, R¹¹, R^{11′}, R¹⁵, R^{15′}, R^{15″}, R^{15″}, R¹⁶, R^{16′}, R²⁰, R^{20′}, R²¹, R^{21′}, R²², and R²³ are each independently selected from H, OH, SH, NH₂, N₃, NO₂, NO, CF₃, CN, C(O)NH₂, C(O)H, C(O)OH, halogen, R^{h}, SR^{h}, S(O)R^{h}, S(O)₂R^{h}, S(O)OR^{h}, S(O)₂OR^{h}, OS(O)R^{h}, OS(O)₂R^{h}, OS(O)OR^{h}, OS(O)₂OR^{h}, OR^{h}, NHR^{h}, N(R^{h})R¹, ⁺N(R^{h})(Rⁱ)R^{j}, P(O)(OR^{h})(ORⁱ), OP(O)(OR^{h})(ORⁱ), SiR^{h}RⁱR^{j}, C(O)R^{h}, C(O)OR^{h}, C(O)N(R^{h})Rⁱ, OC(O)R^{h}, OC(O)OR^{h}, OC(O)N(R^{h})Rⁱ, N(R^{h})C(O)Rⁱ, N(R^{h})C(O)ORⁱ, N(R^{h})C(O)N(Rⁱ)R^{j}, and a water-soluble group, wherein
   R^{h}, Rⁱ, and R^{j} are independently selected from H and optionally substituted (CH₂CH₂O)ₑₑCH₂CH₂X¹³R^{e1}, C₁₋₁₅ alkyl, C₁₋₁₅ heteroalkyl, C₃₋₁₅ cycloalkyl, C₁₋₁₅ heterocycloalkyl, C₅₋₁₅ aryl, or C₁₋₁₅ heteroaryl, one or more of the optional substituents in R^{h}, Rⁱ, and/or R^{j} optionally being a water-soluble group, two or more of R^{h}, Rⁱ, and R^{j} optionally being joined by one or more bonds to form one or more optionally substituted carbocycles and/or heterocycles,
or R⁸+R⁸ and/or R⁹+R^{9′} and/or R¹⁰+R^{10′} and/or R¹¹+R^{11′} and/or R¹⁵+R^{15′} and/or R^{15″}+R^{15′″} and/or R¹⁶+R^{16′} and/or R²⁰+R^{20′} and/or R²¹+R^{21′} are independently selected from ═O, ═S, ═NOR^{h1}, ═C(R^{h1})R^{h2}, and ═NR^{h1}, R^{h1} and R^{h2} being independently selected from H and optionally substituted C₁₋₃ alkyl, two or more of R⁸, R^{8′}, R⁹, R^{9′}, R¹⁰, R^{10′}, R¹¹, R^{11′}, R¹⁵, R^{15′}, R^{15″}, R^{15′″}, R¹⁶, R²⁰, R^{20′}, R²¹, R^{21′}, R²², and R²³ optionally being joined by one or more bonds to form one or more optionally substituted carbocycles and/or heterocycles;
R^{8b} and R^{9b} are independently selected and have the same meaning as R⁸, except that they may not be joined with any other substituent;
one of R⁴ and R^{4′} and one of R¹⁶ and R^{16′} may optionally be joined by one or more bonds to form one or more optionally substituted carbocycles and/or heterocycles;
one of R⁴ and R^{4′} and one of R¹⁶ and R^{16′} may optionally be joined by
one of R², R^{2′}, R³, and R^{3′} and one of R⁵ and R^{5′} may optionally be joined by one or more bonds to form one or more optionally substituted carbocycles and/or heterocycles;
a and b are independently selected from 0 and 1;
the DB moiety does not comprise a DAI, DA2, DAI′, or DA2′ moiety;
ring B in DB1 is a heterocycle;
if X³ in DB1 represents —X^{3a} and X^{3b}— and ring B is aromatic, then two vicinal substituents on said ring B are joined to form an optionally substituted carbocycle or heterocycle fused to said ring B;
if X³ in DB2 represents —X^{3a} and X^{3b}— and ring B is aromatic, then two vicinal substituents on said ring B are joined to form an optionally substituted heterocycle fused to said ring B, an optionally substituted non-aromatic carbocycle fused to said ring B, or a substituted aromatic carbocycle which is fused to said ring B and to which at least one substituent is attached that contains a hydroxy group, a primary amino group, or a secondary amino group, the primary or secondary amine not being a ring atom in an aromatic ring system nor being part of an amide;
if ring A in DB2 is a 6-membered aromatic ring, then substituents on ring B are not joined to form a ring fused to ring B;
two vicinal substituents on ring A in DB8 are joined to form an optionally substituted carbocycle or heterocycle fused to said ring A to form a bicyclic moiety to which no further rings are fused; and ring A in DB9 together with any rings fused to said ring A contains at least two ring heteroatoms.

The above-described Linker Molecules can be conjugated to a cysteine thiol group using thiol-maleimide chemistry, as shown above. In some embodiments, the cytotoxic duocarmycin moiety is a prodrug. For example, the prodrug, vc-seco-DUBA can be conjugated to a self-elimination moiety linked to maleimide linker moiety via a cleavable peptide moiety:

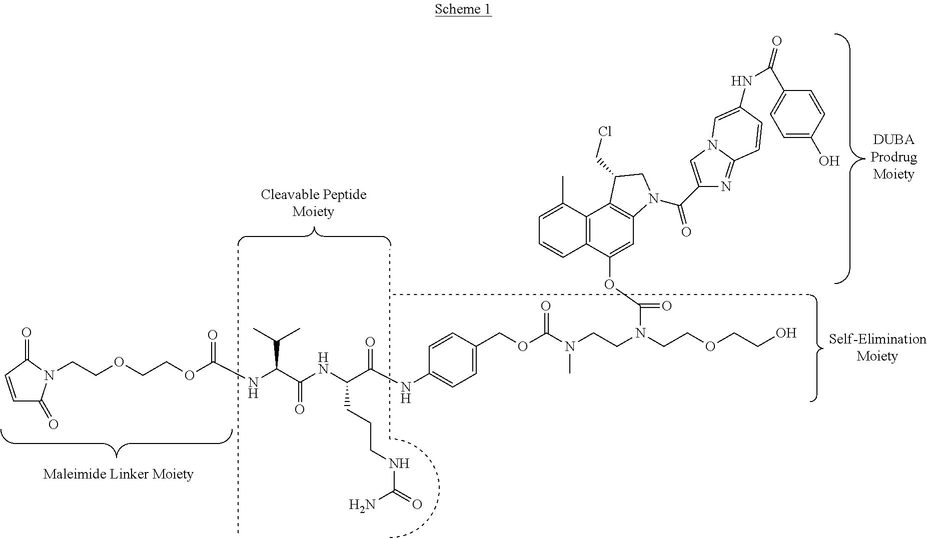 [SMILES: Cc1cccc2c(OC(=O)N(CCOCCO)CCN(C)C(=O)OCc3ccc(NC(=O)[C@H](CCCNC(N)=O)NC(=O)[C@@H](NC(=O)OCCOCCN4C(=O)C=CC4=O)C(C)C)cc3)cc3c(c12)[C@H](CCl)CN3C(=O)c1cn2cc(NC(=O)c3ccc(O)cc3)ccc2n1]

The maleimide linker moiety of the molecule can be conjugated to a thiol group of a cysteine residue of a VL Domain and/or a VH Domain and/or a Constant Domain of the Ab portion of a B7-H3-ADC. Subsequent proteolytic cleavage of the cleavable peptide moiety is followed by the spontaneous elimination of the self-elimination moiety, leading to the release of seco-DUBA, which spontaneously rearranges to form the active drug, DUBA:

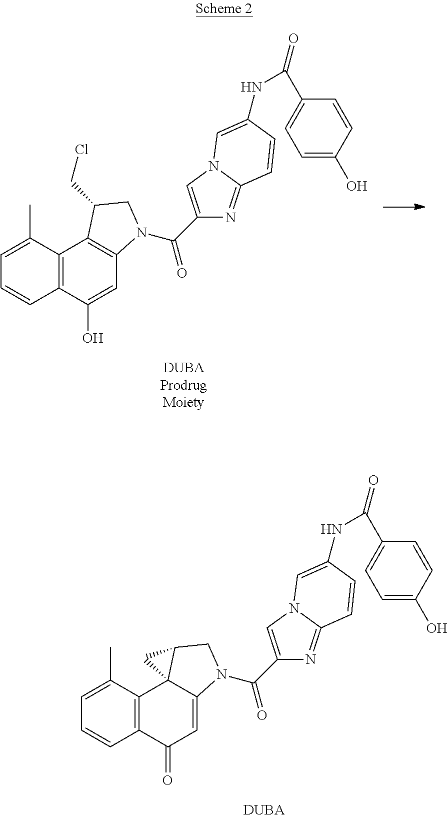 [SMILES: Cc1cccc2c(O)cc3c(c12)[C@H](CCl)CN3C(=O)c1cn2cc(NC(=O)c3ccc(O)cc3)ccc2n1 ; Cc1cccc2c1[C@@]13C[C@@H]1CN(C(=O)c1cn4cc(NC(=O)c5ccc(O)cc5)ccc4n1)C3=CC2=O]

(see, Dokter, W. et al. (2014) “Preclinical Profile of the HER2-Targeting ADC SYD983/SYD985: Introduction of a New Duocarmycin-Based Linker-Drug Platform,” Mol. Cancer Ther. 13(11):2618-2629).

In an exemplary method for the production of B7-H3-duocarmycin drug moiety conjugates, the method of by Elgersma, R.C. et al. (2014) “Design, Synthesis, and Evaluation of Linker-Duocarmycin Payloads: Toward Selection of HER2-Targeting Antibody-Drug Conjugate SYD985,” Mol. Pharmaceut. 12:1813-1835 or that of WO 2011/133039 will be employed. Thus, a thiol-containing group of the VL or VH chain of an anti-B7-H3 antibody or antibody fragment is conjugated to a seco-DUBA or other prodrug through a Maleimide Linker Moiety-Cleavable Peptide Moiety-Self-Elimination Moiety (Scheme 3A):

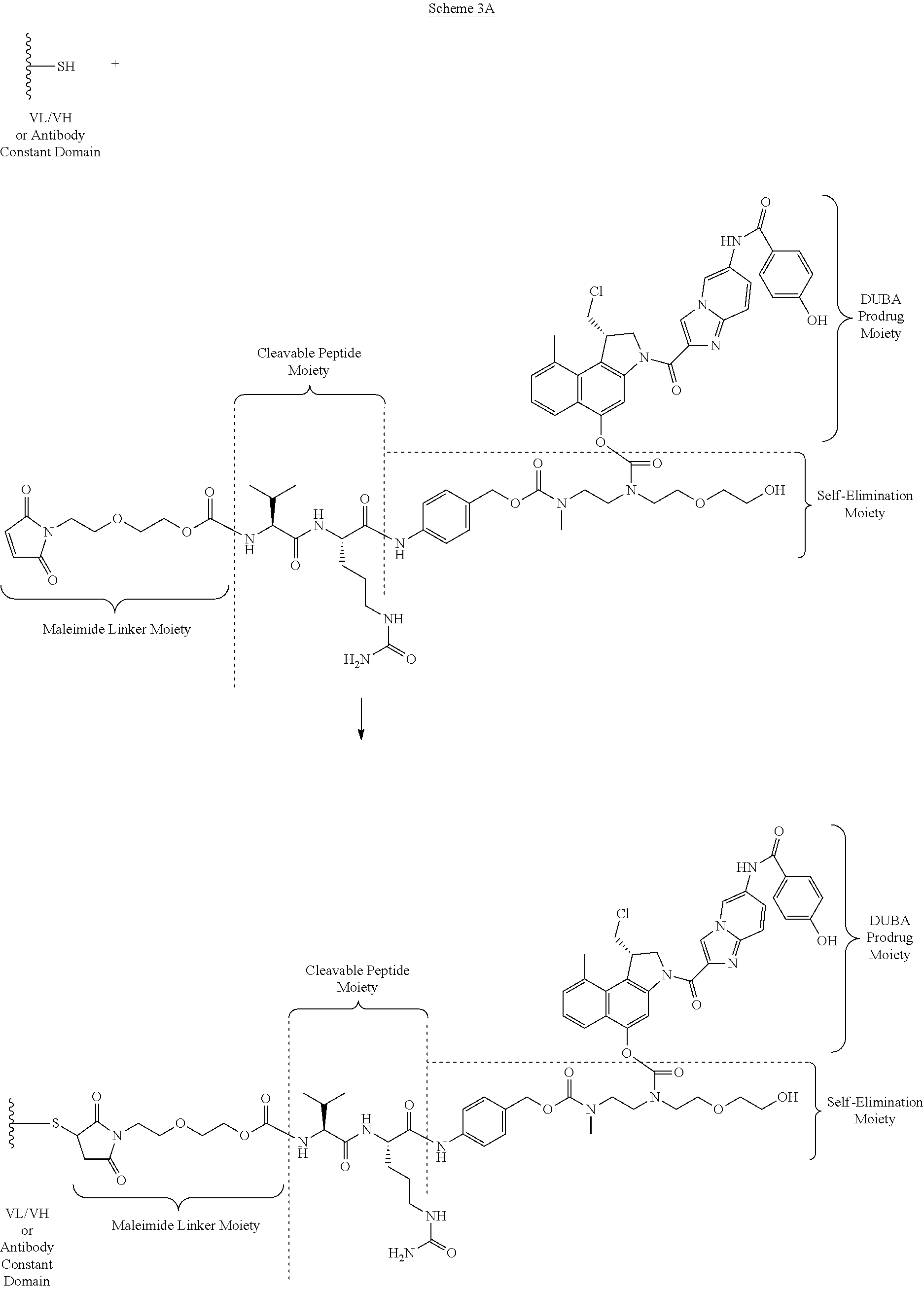

Although the invention is illustrated with regard to a DUBA prodrug, other prodrugs, e.g., CC-1065, may be alternatively employed, as shown in Scheme 3B:

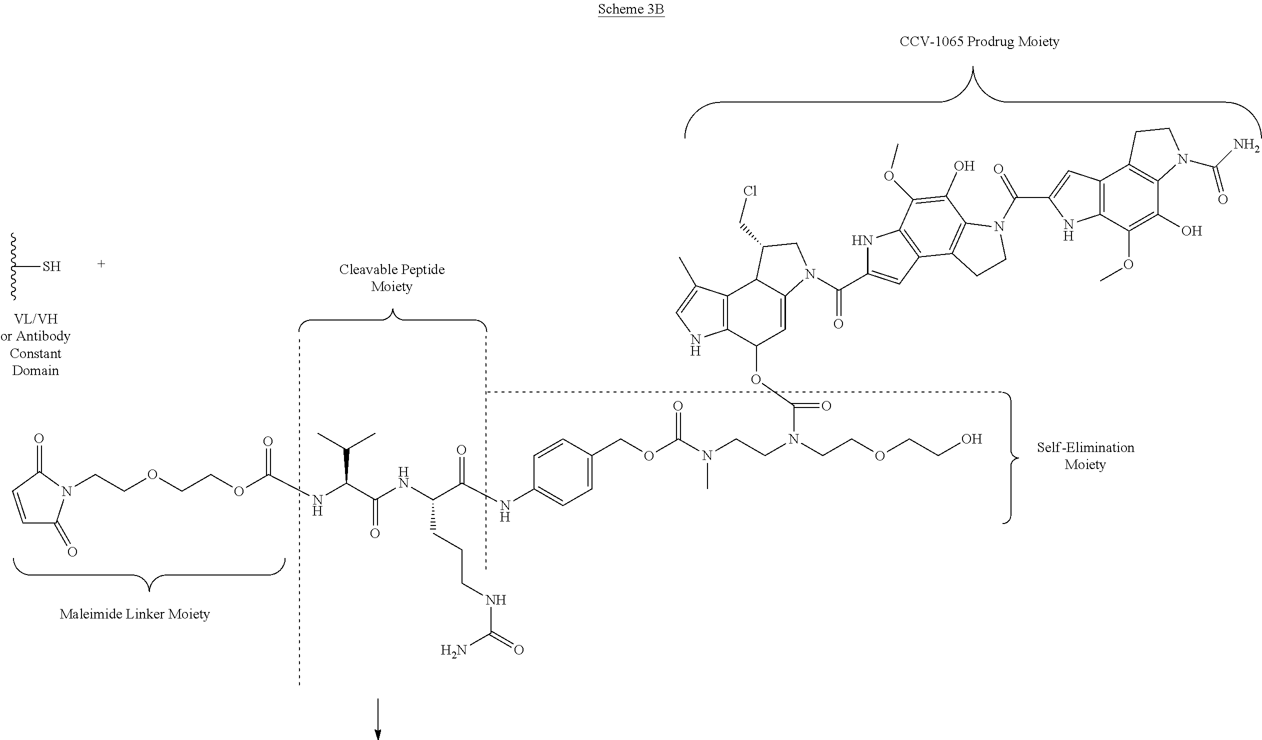

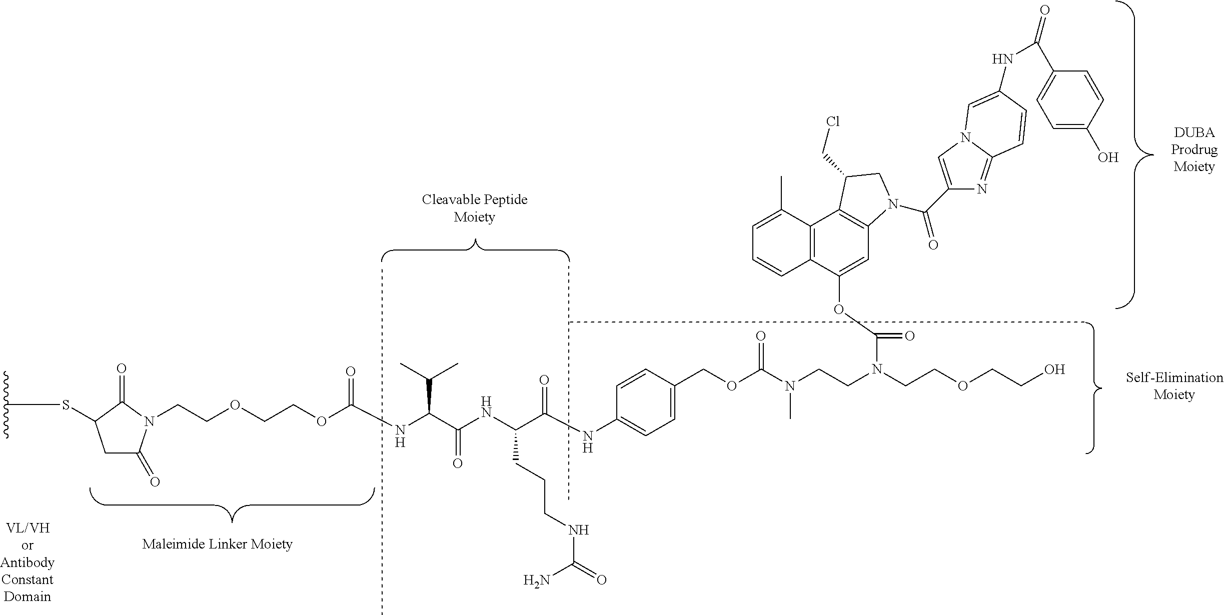 [SMILES: *SC1CC(=O)N(CCOCCOC(=O)N[C@H](C(=O)N[C@@H](CCCNC(N)=O)C(=O)Nc2ccc(COC(=O)N(C)CCN(CCOCCO)C(=O)Oc3cc4c(c5c(C)cccc35)[C@H](CCl)CN4C(=O)c3cn4cc(NC(=O)c5ccc(O)cc5)ccc4n3)cc2)C(C)C)C1=O]

Upon cleavage of the Cleavable Peptide Moiety and elimination of the Self-Elimination Moiety, the Prodrug Moiety is believed to undergo a Winstein spirocyclization to yield the active drug (e.g., DUBA from seco-DUBA as shown in Scheme 3C).

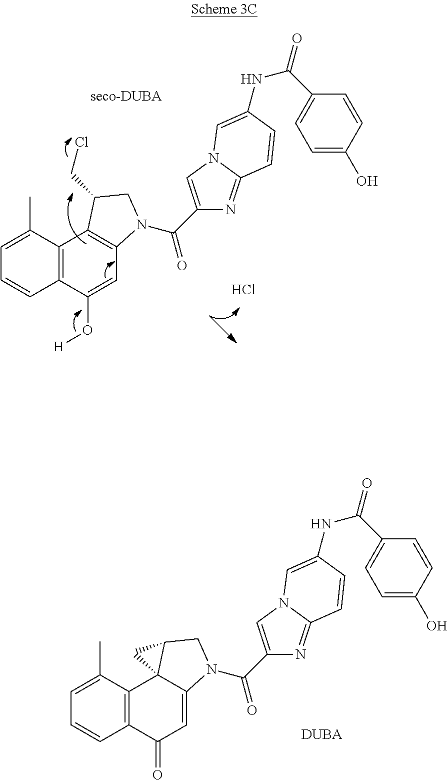

seco-DUBA is prepared from the corresponding DNA-alkylating and DNA-binding moieties (e.g., a 1,2,9,9a-tetrahydrocyclopropa-[c]benzo[e]indole-4-one framework as described by Elgersma, R. C. et al. (2014) “Design, Synthesis, and Evaluation of Linker-Duocarmycin Payloads: Toward Selection of HER2-Targeting Antibody-Drug Conjugate SYD985,” Mol. Pharmaceut. 12:1813-1835 (see, Boger, D. L. et al. (1989) “Total Synthesis and Evaluation of (+)-N-(tert-Butoxycarbonyl)-CBI, (+)-CBI-CDPI1, and (+)-CBI-CDPI2: CC-1065 Functional Agents Incorporating the Equivalent 1,2,9,9a-Tetrahydrocyclopropa[1,2-c]benz[1,2-e]indol-4-one (CBI) Left-Hand Subunit,” J. Am. Chem. Soc. 111:6461-6463; Boger, D. L. et al. (1992) “DNA Alkylation Properties of Enhanced Functional Analogs of CC-1065 Incorporating the 1,2,9,9a-Tetrahydrocyclopropa[1,2-c]benz[1,2-e]indol-4-one (CBI) Alkylation Subunit,” J. Am. Chem. Soc. 114:5487-5496).

Scheme 3D illustrates the invention by showing the synthesis of the DNA-alkylating moiety for DUBA. Thus, o-tolualdehyde (1) and dimethyl succinate (2) are reacted to produce a mixture of acids (3a/3b) through a Stobbe condensation. Ring closure of the mixture of acids may be accomplished with trifluoroacetic anhydride and gave alcohol (4), which is then protected with benzyl chloride to afford benzyl ether (5). The ensuing hydrolysis of the methyl ester group yields the carboxylic acid (6) which is followed by a Curtius rearrangement in a mixture of toluene and tert-butyl alcohol to provide the carbamate (7). Bromination with N-bromosuccinimide give the bromide (8). The bromide (8) is alkylated with (S)-glycidyl nosylate in the presence of potassium tert-butoxide to give epoxide (9). Reaction with n-butyllithium provides a mixture of desired compound (10) and debrominated, rearranged derivative (11). Yields for desired compound (10) are higher when tetrahydrofuran is used as the solvent and the reaction temperature is kept between −25 and −20° C. Under these conditions, desired compound (10) and debrominated, rearranged derivative (11) are obtainable in an approximate 1:1 ratio. Workup with p-toluenesulfonic acid results in conversion of debrominated, rearranged derivative (11) to (7), thereby aiding recovery of desired compound (10). Mesylation of the hydroxyl group in (10) followed by chloride substitution using lithium chloride gives key intermediate (12).

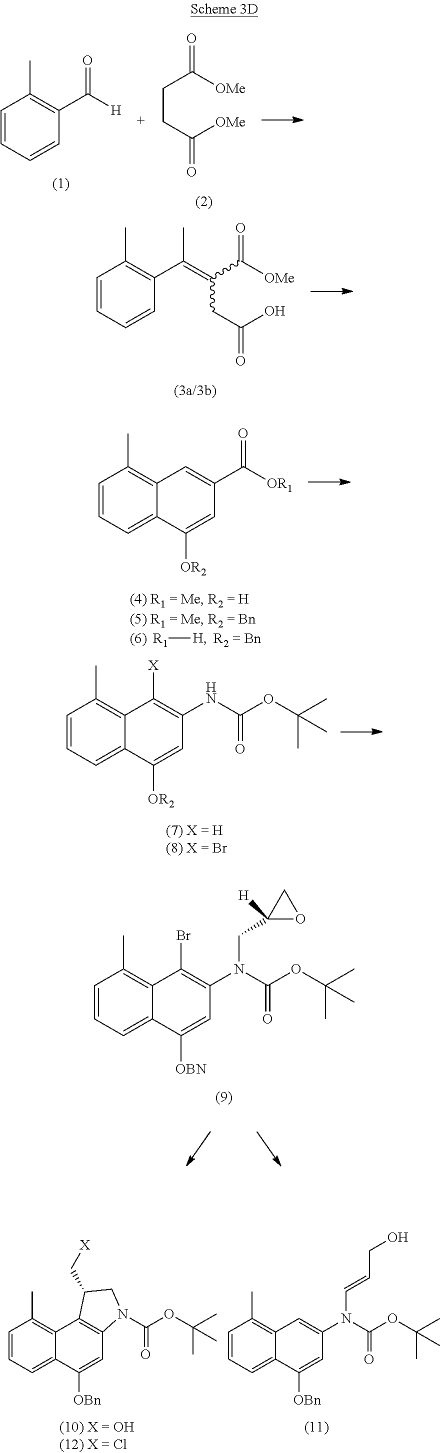

Scheme 3E illustrates the invention by showing the synthesis of the DNA-binding moiety for DUBA. Thus, a Chichibabin cyclization reaction is permitted to proceed between ethyl bromopyruvate (13) and 5-nitropyridin-2-amine (14), thereby obtaining nitro compound (15). Reduction of the nitro group with zinc under acidic conditions gives amine (16). Coupling with methoxymethyl (MOM)-protected 4-hydroxybenzoic acid (17), prepared from methyl 4-hydroxybenzoate through reaction with chloromethyl methyl ether followed by ester hydrolysis (see, WO 2004/080979) gives the ethyl ester (18), which may be hydrolyzed with sodium hydroxide in aqueous 1,4-dioxane to provide acid (19).

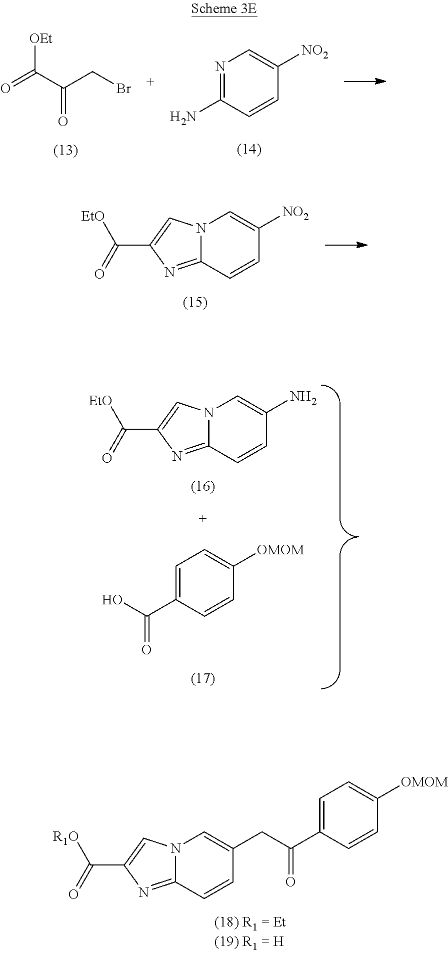

seco-DUBA is then synthesized from DNA-alkylating unit (12) and DNA-binding moiety (19). The tert-butoxycarbonyl (Boc) protective group is removed from (12) under acidic conditions to form the amine (20). EDC-mediated coupling of amine (20) and compound (19) yields protected compound (21), which is then fully deprotected in two consecutive steps (with Pd/C, NH₄HCO₂, MeOH/THF, 3 hours, 90%, to yield (22) and then with HCl, 1,4-dioxane/water, 1 h, 95% to provide seco-DUBA (23) as its HCl salt (Scheme 3F).

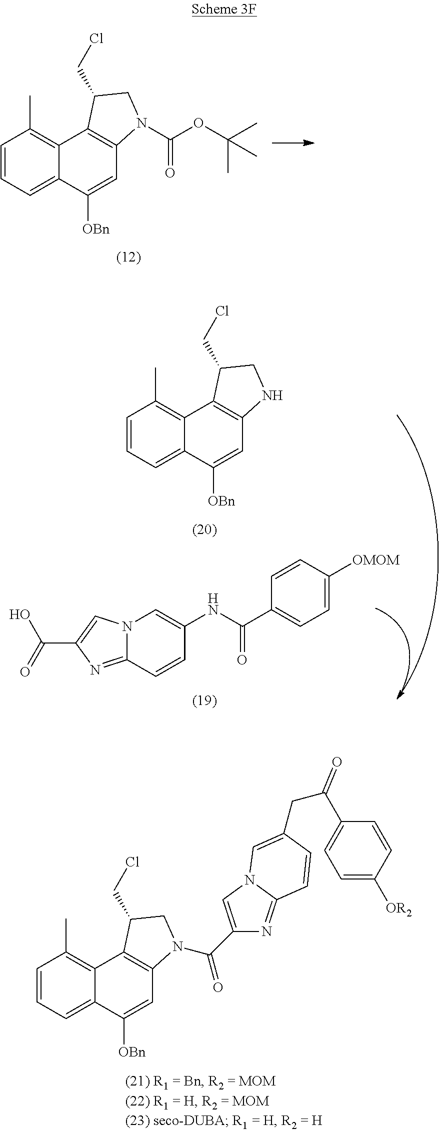

Prodrugs of other drugs, e.g., CC-1065, may be synthesized as described for example in WO 2010/062171.

The Prodrug Moiety may be linked to the other moieties of the ADC according to Scheme 3G. The Maleimide Linker building block was synthesized by starting with a condensation reaction between (24) and 2-(2-aminoethoxy)ethanol (25) to give alcohol (26), which was then converted to reactive carbonate (27) through reaction with 4-nitrophenyl chloroformate. Coupling of (27) to H-Valine-Citrulline-PABA (28), prepared according to Dubowchik, G.M. et al. (2002) “Cathepsin B-Labile Dipeptide Linkers For Lysosomal Release Of Doxorubicin From Internalizing Immunoconjugates: Model Studies Of Enzymatic Drug Release And Antigen-Specific In Vitro Anticancer Activity,” Bioconjugate Chem. 13:855-869) results in the formation of linker (29), which was treated with bis(4-nitrophenyl) carbonate to give activated linker (30).

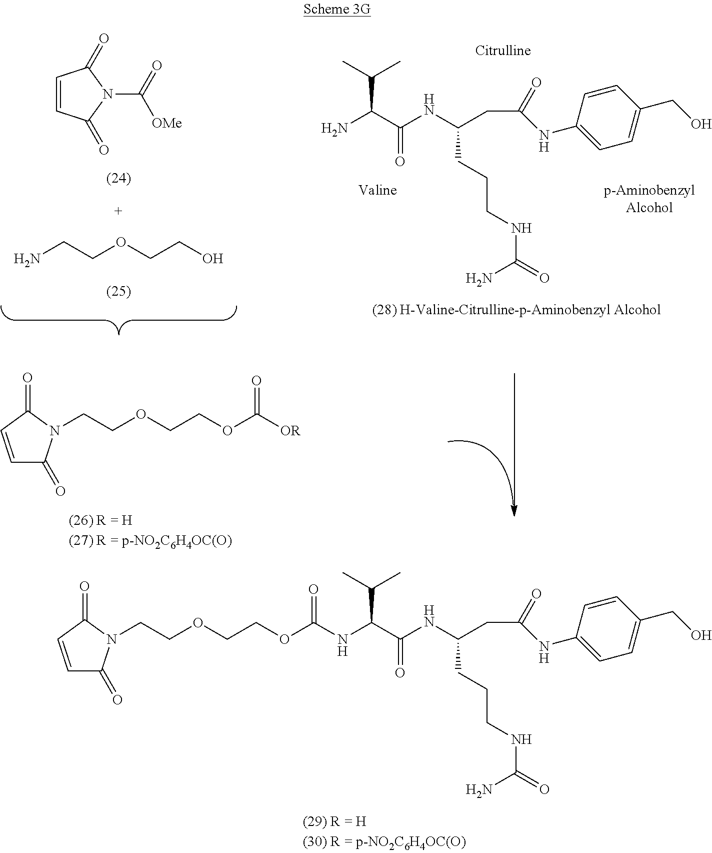

As shown in Scheme 311, seco-DUBA-MOM (22) is modified for conjugation in two steps. Consecutive treatment of (22) with 4-nitrophenyl chloroformate and tent-butyl methyl(2-(methylamino)ethyl)carbamate (31) gives compound (32). Removal of the Boc and MOM protective groups in (32) with trifluoroacetic acid (TFA) provided (33) as its TFA salt.

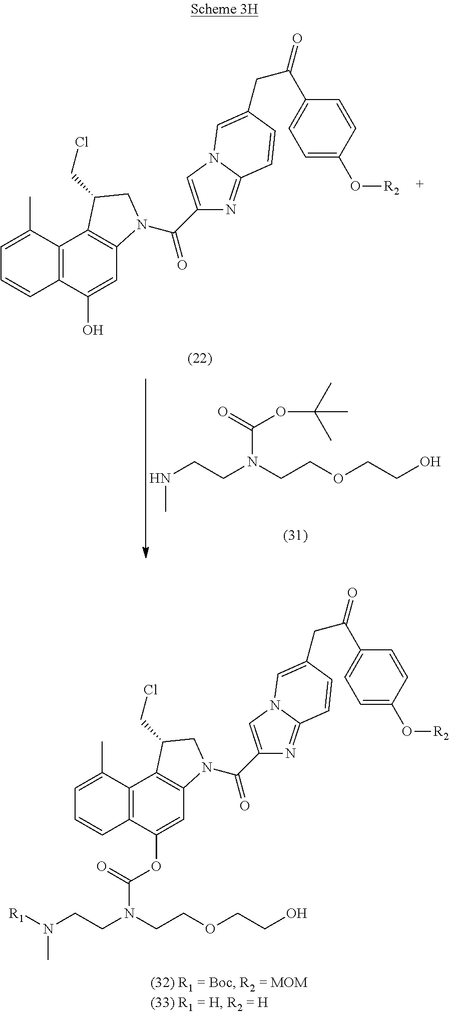

The ADC was synthesized through reaction of activated linker (30) with cyclization spacer-duocarmycin construct (33) under slightly basic conditions. Under these conditions, self-elimination of the cyclization spacer and resulting formation of 3a was suppressed (Scheme 3I).

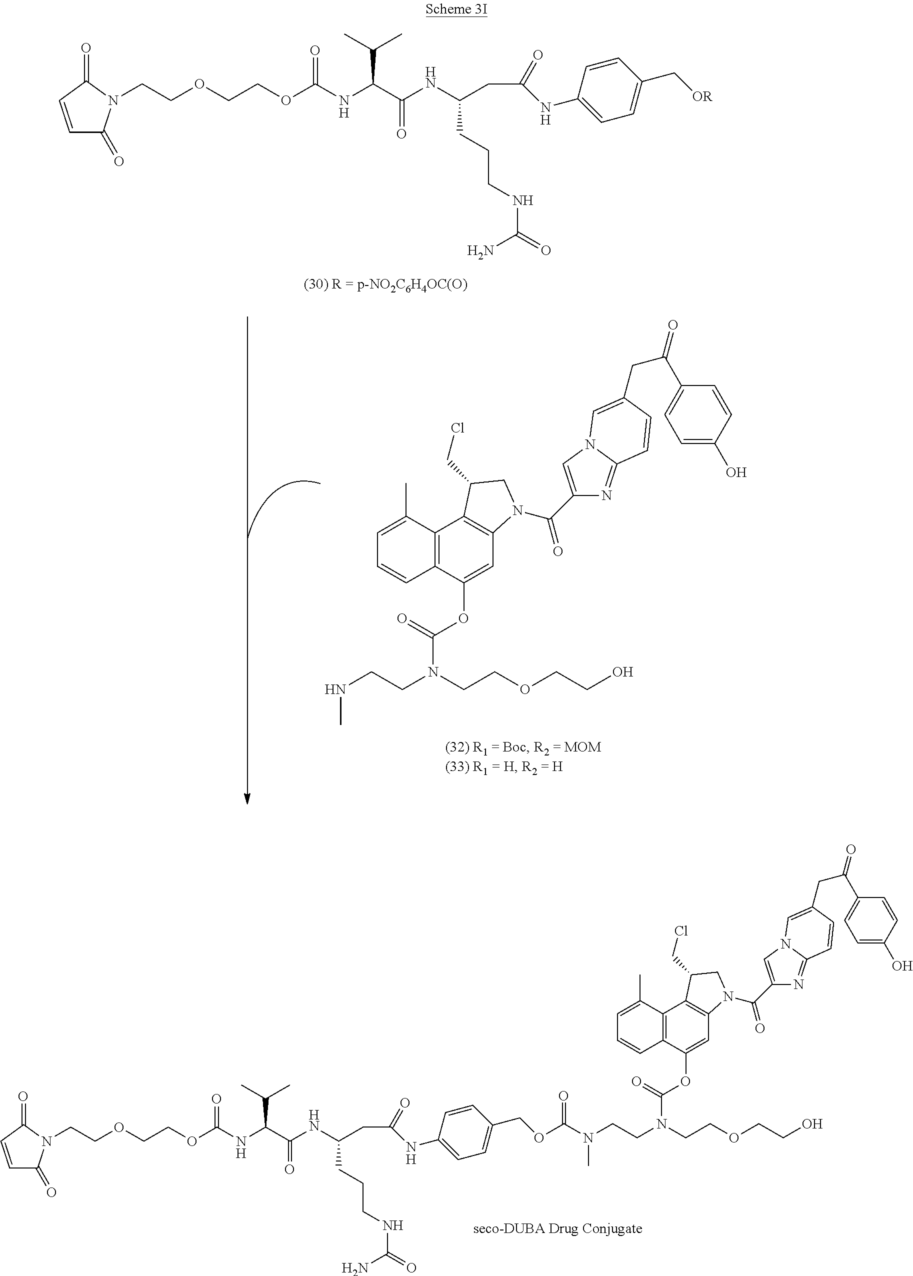

The process generates on average two free thiol groups per mAb leading to a statistical distribution of B7-H3-ADC with an average drug-to-antibody-ratio (DAR) of about two, and low amounts of high-molecular weight species and residual unconjugated duocarmycin moiety.

The order of the steps of the synthesis may be varied as desired. It is specifically contemplated that the method used will be that of Schemes 3A-3I, as described above.

### V. Exemplary PD-1 Binding Molecules

The PD-1 binding molecules of the present invention include bispecific molecules (e.g., bispecific antibodies, bispecific diabodies, etc.), chimeric or humanized antibodies, and such binding molecules having variant Fc Regions. Such PD-1 binding molecules are capable of binding to a continuous or discontinuous (e.g., conformational) portion (epitope) of human PD-1 (CD279). The PD-1 binding molecules of the present invention will preferably also exhibit the ability to bind to PD-1 molecules of one or more non-human species, in particular, primate species (and especially a primate species such as cynomolgus monkey). A representative human PD-1 polypeptide including a 20 amino acid residue signal sequence and the 268 amino acid residue mature protein is provided by NCBI Sequence NP_005009.2 (SEQ ID NO:22).

Antibodies that are specific for PD-1 are known (see, e.g., U.S. Pat. Nos. 7,488,802; 7,521,051; 7,595,048; 8,008,449; 8,354,509; 8,735,553; 8,779,105; 8,900,587; 9,084,776; 10,577,422; PCT Patent Publications WO 2004/056875; WO 2006/121168; WO 2008/156712; WO 2012/135408; WO 2012/145493; WO 2013/014668; WO 2014/179664; WO 2014/194302; WO 2015/112800; and WO2019/246110). Additional desired antibodies may be made by isolating antibody-secreting hybridomas elicited using PD-1 or a peptide fragment thereof. Suitable antibodies include nivolumab ((CAS Reg. No.:946414-94-4, also known as 5C4, BMS-936558, ONO-4538, MDX1106, and marketed as OPDIVO® by Bristol-Myers Squibb); (the amino acid sequence of nivolumab is provided in WHO Drug Information, 2013, Recommended INN: List 69, 27(1):68-69)) and pembrolizumab ((formerly known as lambrolizumab), CAS Reg. No.:1374853-91-4, also known as MK-3475, SCH-900475, and marketed as KEYTRUDA® by Merck); (the amino acid sequence of pembrolizumab is provided in WHO Drug Information, 2014, Recommended INN: List 75, 28(3):407)). The amino acid sequences of the VH and VL domains of these antibodies are provided below.

The PD-1 binding molecules of the present invention may comprise an IgG4 Heavy Chain Constant Region, or a variant IgG1 Heavy Chain Constant Region comprising one or more substitutions which reduce ADCC effector function (e.g., comprising any 1, 2, 3, or 4 of the substitutions: L234A, L235A, D265A, N297Q, and N297G as described above), in lieu of a wild type IgG1 Heavy Chain Constant Region. Such variant Heavy Chain Constant Regions are useful to diminish or abolish the ability of the Fc Domain of an antibody to bind to the FcγRIIIA (CD16a) cellular receptor. Thus, the use of an IgG4 Heavy Chain Constant Region or such variant IgG1 Heavy Chain Constant Region diminishes or abolishes the antibody-dependent cell-mediated cytotoxicity (ADCC) effector function that is associated with the use of antibodies having a wild type IgG1 Fc Domain. The amino acid sequence of the CH2-CH3 Domain of an exemplary human IgG4 is provided above (SEQ ID NO:9).

When the PD-1 binding molecules of the present invention comprise an IgG4 Heavy Chain Constant Region it is preferable to also employ an IgG4 CH1 Domain (SEQ ID NO:6) and an IgG4 Hinge Domain particularly a modified IgG4 Hinge Domain that comprises a Kabat S228P substitution (ESKYGPPCPPCP (SEQ ID NO:7), since that modification stabilizes the IgG4 Hinge Domain.

A. Nivolumab

The amino acid sequence of the VH Domain of nivolumab has the amino acid sequence (SEQ ID NO:36) (CDRH residues are shown underlined):

QVQLVESGGG VVQPGRSLRL DCKASGITFS NSGMHWVRQA
PGKGLEWVAV IWYDGSKRYY ADSVKGRFTI SRDNSKNTLF
LQMNSLRAED TAVYYCATND DYWGQGTLVT VSS

The amino acid sequence of the VL Domain of nivolumab has the amino acid seauence (SEQ ID NO:35) (CDRL residues are shown underlined):

EIVLTQSPAT LSLSPGERAT LSCRASQSVS SYLAWYQQKP
GQAPRLLIYD ASNRATGIPA RFSGSGSGTD FTLTISSLEP
EDFAVYYCQQ SSNWPRTFGQ GTKVEIK

B. Pembrolizumab

The amino acid sequence of the VH Domain of pembrolizumab has the amino acid sequence (SEQ ID NO:34) (CDR_{H} residues are shown underlined):

QVQLVQSGVE VKKPGASVKV SCKASGYTET NYYMYWVRQA
PGQGLEWMGG INPSNGGTNF NEKFKNRVTL TTDSSTTTAY
MELKSLQFDD TAVYYCARRD YRFDMGFDYW GQGTTVTVSS

The amino acid sequence of the VL Domain of pembrolizumab has the amino acid sequence (SEQ ID NO:33) (CDR_{L} residues are shown underlined):

EIVLTQSPAT LSLSPGERAT LSCRASKGVS TSGYSYLHWY
QQKPGQAPRL LIYLASYLES GVPARFSGSG SGTDFTLTIS
SLEPEDFAVY YCQHSRDLPL TFGGGTKVEIK

C. hPD-1 mAb-A

In certain embodiments the PD-1 binding molecule comprises the VL and VL Domains of hPD-1 mAb-A, The amino acid sequence of hPD-1 mAb-A is provided below, and is also disclosed in U.S. Pat. No. 10,577,422 and in PCT Publications WO 2017/062619, WO 2017/019846 and W02019/246110. hPD-1 mAb-A is also known as retifanlimab, MGA012 and INCMGA-00012 (CAS Reg. No.: 2079108-44-2 being co-developed by Incyte and MacroGenics, Inc.). The amino acid sequence of hPD-1 mAb-A is shown below and provided in WHO Drug Information 2019, Recommended INN: List 82, 33(1):611-612.

The amino acid sequence of the VH Domain of hPD-1 mAb-A (SEQ ID NO:32) is shown below (CDRH residues are shown underlined):

QVQLVQSGAE VKKPGASVKV SCKASGYSFT SYWMNWVRQA
PGQGLEWIGV IHPSDSETWL DQKFKDRVTI TVDKSTSTAY
MELSSLRSED TAVYYCAREH YGTSPFAYWG QGTLVTVSS

The amino acid sequence of a Heavy Chain of humanized antibody hPD-1 mAb-A comprising the VL Domain of hPD-1 mAb-A and IgG4 CH1-stabilized H-CH2-CH3 Domains (SEQ ID NO:30) is shown below:

QVQLVQSGAE VKKPGASVKV SCKASGYSFT SYWMNWVRQA
PGQGLEWIGV IHPSDSETWL DQKFKDRVTI TVDKSTSTAY
MELSSLRSED TAVYYCAREH YGTSPFAYWG QGTLVTVSSA
STKGPSVFPL APCSRSTSES TAALGCLVKD YFPEPVTVSW
NSGALTSGVH TFPAVLQSSG LYSLSSVVTV PSSSLGTKTY
TCNVDHKPSN TKVDKRVESK YGPPCPPCPA PEFLGGPSVF
LFPPKPKDTL MISRTPEVTC VVVDVSQEDP EVQFNWYVDG
VEVHNAKTKP REEQFNSTYR VVSVLTVLHQ DWLNGKEYKC
KVSNKGLPSS IEKTISKAKG QPREPQVYTL PPSQEEMTKN
QVSLTCLVKG FYPSDIAVEW ESNGQPENNY KTTPPVLDSD
GSFFLYSRLT VDKSRWQEGN VFSCSVMHEA LHNHYTQKSL
SLSLG

In SEQ ID NO:30, amino acid residues 1-119 correspond to the VH Domain of hPD-1 mAb-A (SEQ ID NO:32), amino acid residues 120-217 correspond to the IgG4 CH1 Domain (SEQ ID NO:4), amino acid residues 218-229 correspond to the stabilized IgG4 Hinge Domain comprising the Kabat S228P substitution (underlined) (SEQ ID NO:7) and amino acid residues 230-445 correspond to the IgG4 CH2-CH3 Domain (SEQ ID NO:9), but lacking the C-terminal lysine residue. The N terminal glutamine of the Heavy Chain may be cyclized to form a pyroglutamic acid. An N-linked glycosylation site is present at Kabat position 296 (double underlined).

The amino acid sequence of the VL Domain of hPD-1 mAb- (SEQ ID NO:31) is shown below (CDRH residues are shown underlined):

EIVLTQSPAT LSLSPGERAT LSCRASESVD NYGMSFMNWF
QQKPGQPPKL LIHAASNQGS GVPSRFSGSG SGTDFTLTIS
SLEPEDFAVY FCQQSKEVPY TFGGGTKVEI K

The amino acid sequence of a Light Chain of humanized antibody hPD-1 mAb-A comprising the VL Domain of hPD-1 mAb-A and a CL Kappa Domain (SEQ ID NO:29) is shown below:

EIVLTQSPAT LSLSPGERAT LSCRASESVD NYGMSEMNWE
QQKPGQPPKL LIHAASNQGS GVPSRFSGSG SGTDFTLTIS
SLEPEDFAVY FCQQSKEVPY TFGGGTKVEI KRTVAAPSVF
IFPPSDEQLK SGTASWVVLL NNFYPREAKV QWKVDNALQS
GNSQESVTEQ DSKDSTYSLS STLTLSKADY EKHKVYACEV
THQGLSSPVT KSFNRGEC

In SEQ ID NO:29, amino acid residues 1-111 correspond to the VL Domain hPD-1 mAb-A (SEQ ID NO:31), and amino acid residues 112-218 correspond to the Light Chain kappa constant region (SEQ ID NO:1).

D. PD-1 X LAG-3 Bispecific Molecule

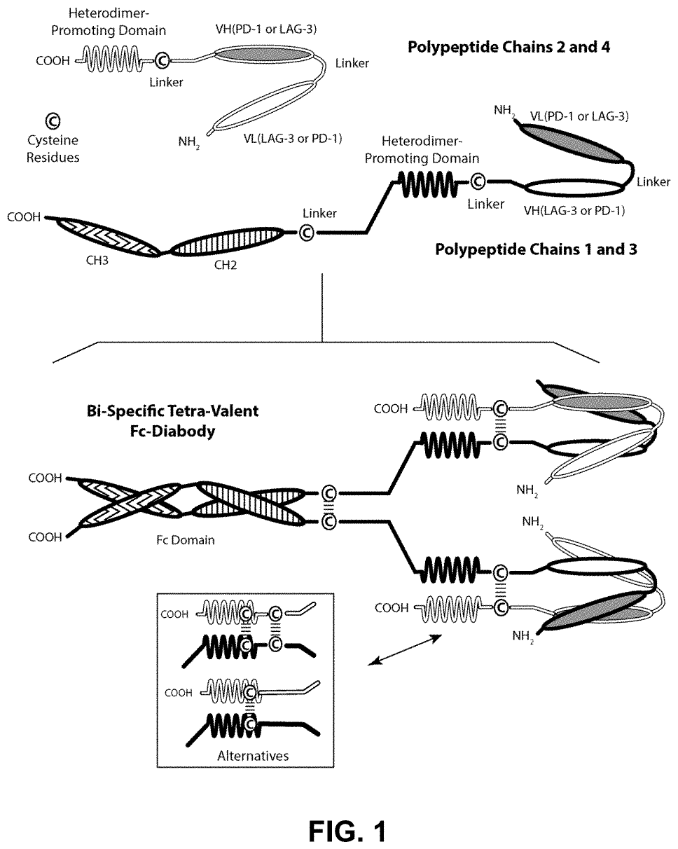

In certain embodiments, the PD-1 binding molecule is a bispecific molecule that binds to PD-1 and LAG-3. PD-1 X LAG-3 bispecific molecules for use in the treatment of cancer and/or a disease associated with a pathogen are described in PCT Publication Nos. WO 2015/200119, WO 2017/025498, WO 2018/083087, WO 2018/185043, WO 2018/134279, and WO 2018/217940. In particular embodiments, the bispecific molecule is a PD-1 X LAG-3 bispecific diabody. PD-1 X LAG-3 bispecific diabodies having novel PD-1 and LAG-3 binding domains and exemplary activity as described in WO 2017/019846. In a specific embodiment the diabody is “PD-1 X LAG-3 BD”. PD-1 X LAG-3 BD is a four chain, Fc Region-containing diabody having two binding sites specific for PD-1, two binding sites specific for LAG-3, an Fc Region, and cysteine-containing E/K-coil Heterodimer-Promoting Domains. The general structure of the PD-1 X LAG-3 BD is provided in FIG. 1. PD-1 X LAG-3 BD comprises a VL and VH Domain of a humanized antibody that binds to PD-1 and also a VL and VH Domain of a humanized antibody that binds to LAG-3. Thus, PD-1 X LAG-3 BD is capable of specifically binding to an epitope of PD-1 and to an epitope of LAG-3.

PD-1 X LAG-3 BD comprises four polypeptide chains. The first and third polypeptide chains of PD-1 X LAG-3 BD comprise, in the N-terminal to C-terminal direction: an N-terminus, a VL Domain of a monoclonal antibody capable of binding to LAG-3 (SEQ ID NO:45; bolded and underlined in SEQ ID NO:37 below); an intervening linker peptide (Linker 1: GGGSGGGG (SEQ ID NO:10)); a VH Domain of a monoclonal antibody capable of binding to PD-1 (SEQ ID NO:32; bolded and double underlined in SEQ ID NO:37 below); a cysteine-containing intervening linker peptide (Linker 2: GGCGGG (SEQ ID NO:11)); a cysteine-containing Heterodimer-Promoting (E-coil) Domain (EVAACEK-EVAALEK-EVAALEK-EVAALEK (SEQ ID NO:12)); an intervening linker peptide (Linker 3) comprising a stabilized IgG4 hinge region (SEQ ID NO:7); a variant IgG4 CH2-CH3 Domain comprising substitutions M252Y/S254T/T256E and lacking the C-terminal residue (SEQ ID NO:14); and a C-terminus.

The amino acid sequence of the first and third polypeptide chains of PD-1 X LAG-3 BD is (SEQ ID NO:37):

DIQMTQSPSS LSASVGDRVT ITCRASQDVS SVVAWYQQKP
GKAPKLLIYS ASYRYTGVPS RFSGSGSGTD FTLTISSLQP
EDFATYYCQQ HYSTPWTFGG GTKLEIKGGG SGGGG

[symbol]

[symbol]

[symbol]

[symbol]

[symbol]

[symbol]

[symbol]

[symbol]

[symbol]

[symbol]

[symbol]

[symbol]

[symbol]

GGCGGG
EVAACEKEVA ALEKEVAALE KEVAALEKES KYGPPCPPCP
APEFLGGPSV FLFPPKPKDT LYITREPEVT CVVVDVSQED
PEVQFNWYVD GVEVHNAKTK PREEQFNSTY RVVSVLTVLH
QDWLNGKEYK CKVSNKGLPS SIEKTISKAK GQPREPQVYT
LPPSQEEMTK NQVSLTCLVK GEYPSDIAVE WESNGQPENN
YKTTPPVLDS DGSFFLYSRL TVDKSRWQEG NVFSCSVMHE
ALHNHYTQKS LSLSLG

The second and fourth polypeptide chains of PD-1 X LAG-3 BD comprise, in the N-terminal to C-terminal direction: an N-terminus, a VL Domain of a monoclonal antibody capable of binding to PD-1 (SEQ ID NO:31; bolded and underlined in SEQ ID NO:38 below); an intervening linker peptide (Linker 1: GGGSGGGG (SEQ ID NO:10)); a VH Domain of a monoclonal antibody capable of binding LAG-3 (SEQ ID NO:46; bolded and double-underlined in SEQ ID NO:38 below); a cysteine-containing intervening linker peptide (Linker 2: GGCGGG (SEQ ID NO:11)); a cysteine-containing Heterodimer-Promoting (K-coil) Domain (KVAACKE-KVAALKE-KVAALKE-KVAALKE (SEQ ID NO:13); and a C-terminus.

The amino acid sequence of the second and fourth polypeptide chains of PD-1 X LAG-3 BD is (SEQ ID NO:38):

EIVLTQSPAT LSLSPGERAT LSCRASESVD NYGMSFMNWE
QQKPGQPPKL LIHAASNQGS GVPSRFSGSG SGTDFTLTIS
SLEPEDFAVY FCQQSKEVPY TFGGGTKVEI
KGGGSGGGG

[symbol]

[symbol]

[symbol]

[symbol]

[symbol]

[symbol]

[symbol]

[symbol]

[symbol]

[symbol]

[symbol]

[symbol]

GGC GGGKVAACKE KVAALKEKVA ALKEKVAALK E

### VI. Methods of Production

The binding molecules of the invention (e.g., B7-H3-ADC, hPD-1 mAb A, and PD-1 X LAG-3 BD) can be may be made recombinantly and expressed using any method known in the art for the production of recombinant proteins. For example, nucleic acids encoding the polypeptide chains of such binding molecules can be constructed, introduced into an expression vector, and expressed in suitable host cells. The binding molecules may be recombinantly produced in bacterial cells (e.g., E. coli cells), or eukaryotic cells (e.g., CHO, 293E, COS, NSO cells). In addition, the binding molecules can be expressed in a yeast cell such as Pichia, or Saccharomyces.

To produce the binding molecules (e.g., B7-H3-ADC, hPD-1 mAb A, and PD-1 X LAG-3 BD), one or more polynucleotides encoding the molecule may be constructed, introduced into an expression vector, and then expressed in suitable host cells. Standard molecular biology techniques are used to prepare the recombinant expression vector, transfect the host cells, select for transformants, culture the host cells and recover the molecules (See, for example, the techniques described in Green, M. R. et al., (2012), MOLECULAR CLONING, A LABORATORY MANUAL, 4th Ed., Cold Spring Harbor Laboratory, Cold Spring Harbor, N.Y. and Ausubel et al. eds., 1998, CURRENT PROTOCOLS IN MOLECULAR BIOLOGY, John Wiley & Sons, NY). The expression vector(s) should have characteristics that permit replication of the vector in the host cell. The vector should also have promoter and signal sequences necessary for expression in the host cells. Such sequences are well known in the art. In addition to the nucleic acid sequence(s) encoding such binding molecules, the recombinant expression vectors may carry additional sequences, such as sequences that regulate replication of the vector in host cells (e.g., origins of replication) and selectable marker genes. Another method that may be employed is to express the gene sequence in plants (e.g., tobacco) or a transgenic animal. Suitable methods useful for expressing such binding molecules recombinantly in plants or milk have been disclosed (see, for example, Peeters et al. (2001) “Production Of Antibodies And Antibody Fragments In Plants,” Vaccine 19:2756; U.S. Pat. No. 5,849,992; and Pollock et al. (1999) “Transgenic Milk As A Method For The Production Of Recombinant Antibodies,” J. Immunol Methods 231:147-157).

Once a binding molecule has been recombinantly expressed, it may be purified from inside or outside (such as from culture media) of the host cell by any method known in the art for purification of polypeptides or polyproteins. Methods for isolation and purification commonly used for antibody purification (e.g., antibody purification schemes based on antigen selectivity) may be used for the isolation and purification of such molecules and are not limited to any particular method. For example, by for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, e.g., ion exchange, affinity, particularly by affinity for the specific antigen (optionally after Protein A selection where the PD-1 X LAG-3 BD comprises an Fc Region), sizing column chromatography, hydrophobic, gel filtration, reverse-phase, and adsorption (Marshak et al. (1996) STRATEGIES FOR PROTEIN PURIFICATION AND CHARACTERIZATION: A Laboratory Course Manual. (Eds.), Cold Spring Harbor Laboratory Press, Cold Spring Harbor, N.Y.).

### VII. Pharmaceutical Compositions

A B7-H3-ADC and PD-1 binding molecules of the invention (e.g., hPD-1 mAb-A and/or PD-1 X LAG-3 BD) can be formulated as compositions. The compositions of the invention include bulk drug compositions useful in the manufacture of pharmaceutical compositions (e.g., impure or non-sterile compositions) and pharmaceutical compositions (i.e., compositions that are suitable for administration to a subject or patient) that can be used in the preparation of unit dosage forms. Such compositions comprise a prophylactically or therapeutically effective amount of a B7-H3-ADC of the present invention, one or more PD-1 binding molecules, or a combination thereof, and one or more pharmaceutically acceptable carrier(s) and may optionally additionally include one or more additional therapeutic agents. The pharmaceutical compositions may be supplied, for example, as an aqueous solution, or a dry lyophilized powder or water-free concentrate specifically adapted for reconstitution with such a pharmaceutically acceptable carrier or reconstituted with such a carrier.

As used herein, the term “pharmaceutically acceptable carrier” means a diluent, solvent, dispersion media, antibacterial and antifungal agents, excipient, or vehicle approved by a regulatory agency of the Federal or a state government or listed in the U.S. Pharmacopeia or other generally recognized pharmacopeia as being suitable for administration to animals, and more particularly to humans. Such pharmaceutical carriers can be sterile liquids, such as water and oils, including those of petroleum, animal, vegetable or synthetic origin. Saline solutions and aqueous dextrose and glycerol solutions can also be employed as liquid carriers, particularly for injectable solutions. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions are supplied either separately or mixed together in a dose form, for example, as a dry lyophilized powder or water-free concentrate, or as an aqueous solution in a hermetically sealed container such as a vial, ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection, saline or other diluent can be provided so that the ingredients may be mixed prior to administration.

### VIII. Pharmaceutical Kits

The invention also provides a pharmaceutical pack or kit comprising one or more containers containing a pharmaceutical composition or pharmaceutical compositions and instructional material (e.g., a notice, package insert, instruction, etc.). Additionally, one or more other prophylactic or therapeutic agents useful for the treatment of a disease can also be included in the pharmaceutical kit. The containers of such pharmaceutical kits may, for example, comprise one or more hermetically sealed vials, ampoules, sachets, etc., indicating the quantity of active agent contained therein. Where the composition is to be administered by infusion, the container may be an infusion bottle, bag, etc. containing a sterile pharmaceutical-grade solution (e.g., water, saline, a buffer, etc.). Where the compositions are to be administered by injection, the pharmaceutical kit may contain an ampoule of sterile water, saline or other diluent for injection, so as to facilitate the mixing of the components of the pharmaceutical kit for administration to a subject (e.g., a human patient or other mammal). In certain embodiments, a pharmaceutical pack or kit comprises a B7-H3-ADC pharmaceutical composition and instructional material. In other embodiments, a pharmaceutical pack or kit comprises a B7-H3-ADC pharmaceutical composition, and PD-1 binding molecule composition, and instructional material.

In one embodiment, a B7-H3-ADC and/or the PD-1 binding molecule (e.g., hPD-1 mAb-A and/or PD-1 X LAG-3 BD) of such kit is/are supplied as a dry sterilized lyophilized powder or water-free concentrate in a hermetically sealed container and can be reconstituted, e.g., with water, saline, or other diluent to the appropriate concentration for administration to a subject. In another embodiment, a B7-H3-ADC and/or the PD-1 binding molecule (e.g., hPD-1 mAb-A and/or PD-1 X LAG-3 BD) of such kit is supplied as an aqueous solution in a hermetically sealed container and can be diluted, e.g., with water, saline, or other diluent, to the appropriate concentration for administration to a subject. The kit can further comprise one or more other prophylactic and/or therapeutic agents useful for the treatment of cancer, in one or more containers; and/or the kit can further comprise one or more cytotoxic antibodies that bind one or more cancer antigens associated with cancer. In certain embodiments, the other prophylactic or therapeutic agent is a chemotherapeutic. In other embodiments, the prophylactic or therapeutic agent is a biological or hormonal therapeutic.

The included instructional material of the pharmaceutical kits may, for example, be of a content and format prescribed by a governmental agency regulating the manufacture, use or sale of pharmaceuticals or biological products, and may indicate approval by the agency of the manufacture, sale or use of the pharmaceutical composition for human administration and/or for human therapy. The instructional material may, for example provide information relating to the contained dose of the pharmaceutical composition, modes of how it may be administered, etc. Such instructions may further provide information relating to the dose and administration of one or more pharmaceutical composition that are not provided in the kit.

Thus, for example, the included instructional material of the pharmaceutical kits may instruct that the provided pharmaceutical compositions are to be administered in combination with an additional agent which may be provided in the same pharmaceutical kit or in a separate pharmaceutical kit. Such instructional material may instruct that the provided B7-H3-ADC pharmaceutical composition comprises, or is to be reconstituted to administer a dose of about 0.5 mg/kg to about 2 mg/kg, about 2 mg/kg to about 3 mg/kg, about 2 mg/kg to about 2.25 mg/kg, about 2.25 mg/kg to about 2.5 mg/kg, about 2.5 mg/kg to about 2.75 mg/kg, about 2.75 mg/kg to about 3 mg/kg, about 3 mg/kg to about 4 mg/kg, about 3 mg/kg to about 3.25 mg/kg, about 3.25 mg/kg to about 3.5 mg/kg, about 3.5 mg/kg to about 3.75 mg/kg, about 3.75 mg/kg to about 4 mg/kg, about 4 mg/kg to about 5 mg/kg, about 4 mg/kg to about 4.25 mg/kg, about 4.25 mg/kg to about 4.5 mg/kg, about 4.5 mg/kg to about 4.75 mg/kg, or about 5 mg/kg. Such instructional material may instruct that the provided B7-H3-ADC pharmaceutical composition comprises, or is to be reconstituted to administer a dose of about 0.5 mg/kg, about 1 mg/kg, about 1.5 mg/kg, about 2 mg/kg, about 2.25 mg/kg, about 2.5 mg/kg, about 2.75 mg/kg, about 3 mg/kg, about 3.25 mg/kg, about 3.5 mg/kg, about 3.75 mg/kg, about 4 mg/kg, about 4.25 mg/kg, about 4.5 mg/kg, about 4.75 mg/kg, or about 5 mg/kg. Such instructional material may instruct that the provided B7-H3-ADC pharmaceutical composition is to be administered once about every 2 weeks, once about every 3 weeks, about once every 4 weeks, or more or less often. Such instructional material may instruct that a PD-1 binding molecule pharmaceutical composition is also administered. Such instructional material may instruct that the PD-1 binding molecule pharmaceutical composition comprises, or is to be reconstituted to administer a flat dose of about 120 mg to about 800 mg. Such instructional material may instruct that the PD-1 binding molecule pharmaceutical composition comprises, or is to be reconstituted to administer a flat dose about 120 mg, about 200 mg, about 240 mg, about 300 mg, about 375 mg, about 400 mg, about 480 mg, about 500 mg, about 600 mg, or about 800 mg. Such instructional material may instruct that the PD-1 binding molecule pharmaceutical composition comprises, or is to be reconstituted to administer a dose of about 1 mg/kg to about 10 mg/kg, about 1 mg/kg to about 5 mg/kg, or about 5 mg/kg to about 10 mg/kg. Such instructional material may instruct that the PD-1 binding molecule pharmaceutical composition comprises, or is to be reconstituted to administer a dose about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg. Such instructional material may instruct that the provided pharmaceutical composition comprises, or is to be reconstituted to comprise, a single dose, or more than one dose (e.g., 2 doses, 4 doses, 6 doses, 12 doses, 24 doses, etc.). Such instructional material may instruct that the PD-1 binding molecule pharmaceutical composition is to be administered once about every 2 weeks, once about every 3 weeks, about once every 4 weeks, or more or less often. The included instructional material of the pharmaceutical kits may combine any set of such information (e.g., it may instruct that the provided B7-H3-ADC-containing pharmaceutical composition comprises, or is to be reconstituted to comprise, a dose of about 3 mg/ml, and that such dose is to be administered about once every 3 weeks; it may instruct that the provided pharmaceutical composition comprises, or is to be reconstituted to comprise, a dose of about 3.5 mg/kg, and that such dose is to be administered once about every 3 weeks; etc., and/or it may instruct that a hPD-1 mAb-A-containing pharmaceutical composition comprises, or is to be reconstituted to comprise, a flat dose of about 375 mg, and that such dose is to be administered once about every 3 weeks; etc.), and/or it may instruct that a PD-1 X LAG-3 BD-containing pharmaceutical composition comprises, or is to be reconstituted to comprise, a flat dose of about 300 mg or about 600 mg, and that such dose is to be administered once about every 2 weeks or once about every 3 weeks; etc.). Such instructional material may instruct regarding the mode of administration of the included pharmaceutical composition, for example that it is to be administered by intravenous (IV) infusion. The included instructional material of the pharmaceutical kits may instruct regarding the duration or timing of such administration, for example that the included pharmaceutical composition is composition is to be administered by intravenous (IV) infusion over a period of about 60 minutes, about 30-240 minutes, a period of about 30-90 minutes, etc.

The included instructional material of the pharmaceutical kits may instruct regarding the appropriate or desired use of the included pharmaceutical composition, for example instructing that such pharmaceutical composition is to be administered for the treatment of cancer in which B7-H3 is expressed. Such cancer may be an adrenal gland cancer, an AIDS-associated cancer, an alveolar soft part sarcoma, an astrocytic tumor, an anal cancer (e.g., SCAC), a bladder cancer, a bone cancer, a brain and spinal cord cancer, a metastatic brain tumor, a B-cell cancer, a breast cancer (e.g., HER2+ breast cancer or TNBC), a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, a gastric cancer, a gestational trophoblastic disease, a germ cell tumor, a head and neck cancer, a glioblastoma, a hematological malignancy, a hepatocellular carcinoma, an islet cell tumor, a Kaposi's Sarcoma, a kidney cancer, a leukemia (e.g., an acute myeloid leukemia), a liposarcoma/malignant lipomatous tumor, a liver cancer, a lymphoma, a lung cancer (e.g., NSCLC), a medulloblastoma, a melanoma, a meningioma, a mesothelioma pharyngeal cancer, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a pheochromocytoma, a pituitary tumor, a prostate cancer (e.g., mCRPC), a posterior uveal melanoma, a renal metastatic cancer, a rhabdoid tumor, a rhabdomyosarcoma, a sarcoma, a skin cancer, a small round blue cell tumor of childhood (including neuroblastoma and rhabdomyosarcoma), a soft-tissue sarcoma, a squamous cell cancer (e.g., SCCHN), a stomach cancer, a synovial sarcoma, a testicular cancer, a thymic carcinoma, a thymoma, a thyroid cancer (e.g., a thyroid metastatic cancer), and a uterine cancer.

IX. Uses of a B7-H3-ADC of the Invention

A B7-H3-ADC of the present invention can be used optionally in combination with a PD-1 binding molecule of the present invention to treat or prevent a variety of disorders, including cancer, particularly a cancer in which B7-H3 is expressed. Accordingly, the present invention provides methods of treating cancer, such methods comprising administering to a subject in need thereof a B7-H3-ADC of the present invention optionally in combination with a PD-1 binding molecule of the present invention. As used herein, the term “subject” refers to a human (i.e., a human patient) or other mammal. Exemplary dosing regimens for administering such therapy to a subject in need thereof are provided herein.

The cancers that may be treated either with a B7-H3-ADC alone or by the combination of a B7-H3-ADC and a PD-1 binding molecule of the present inventions include: an adrenal gland cancer, an AIDS-associated cancer, an alveolar soft part sarcoma, an astrocytic tumor, an anal cancer (e.g., SCAC), a bladder cancer, a bone cancer, a brain and spinal cord cancer, a metastatic brain tumor, a B-cell cancer, a breast cancer (e.g., HER2+ breast cancer or TNBC), a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, a gastric cancer, a gestational trophoblastic disease, a germ cell tumor, a head and neck cancer, a glioblastoma, a hematological malignancy, a hepatocellular carcinoma, an islet cell tumor, a Kaposi's Sarcoma, a kidney cancer, a leukemia (e.g., an acute myeloid leukemia), a liposarcoma/malignant lipomatous tumor, a liver cancer, a lymphoma, a lung cancer (e.g., NSCLC), a medulloblastoma, a melanoma, a meningioma, a mesothelioma pharyngeal cancer, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a pheochromocytoma, a pituitary tumor, a prostate cancer (e.g., mCRPC), a posterior uveal melanoma, a renal metastatic cancer, a rhabdoid tumor, a rhabdomyosarcoma, a sarcoma, a skin cancer, a small round blue cell tumor of childhood (including neuroblastoma and rhabdomyosarcoma), a soft-tissue sarcoma, a squamous cell cancer (e.g., SCCHN), a stomach cancer, a synovial sarcoma, a testicular cancer, a thymic carcinoma, a thymoma, a thyroid cancer (e.g., a thyroid metastatic cancer), and a uterine cancer.

In particular, a B7-H3-ADC of the present invention may be used optionally in combination with PD-1 binding molecules of the present invention in the treatment of: prostate cancer (including mCRPC), anal cancer (including SCAC), breast cancer (including HER2+ breast cancer and/or TNBC), head and neck cancer (including SCCHN), and lung cancer (including NSCLC).

In certain embodiments, a B7-H3-ADC of the present invention is administered optionally in combination with a PD-1 binding molecule of the present invention as a first-line therapy for treatment of cancer. In other embodiments, a B7-H3-ADC of the present invention is administered optionally in combination with a PD-1 binding molecule of the present invention after one or more prior lines of therapy. In still other embodiments, a B7-H3-ADC of the present invention can be employed optionally in combination with a PD-1 binding molecule of the present invention as an adjuvant therapy at the time of, or after surgical removal of a tumor in order to delay, suppress or prevent the development of metastasis. A B7-H3-ADC of the present invention can also be administered optionally in combination with a PD-1 binding molecule of the present invention before surgery (e.g., as a neoadjuvant therapy) in order to decrease the size of the tumor and thus enable or simplify such surgery, spare tissue during such surgery, and/or decrease any resulting disfigurement.

The invention specifically encompasses administering a B7-H3-ADC, optionally in combination with a PD-1 binding molecule, in further combination with one or more other therapies known to those skilled in the art for the treatment or prevention of cancer, including but not limited to, current standard and experimental chemotherapies, hormonal therapies, biological therapies, immunotherapies, radiation therapies, or surgery. In some embodiments, a B7-H3-ADC, optionally in combination with a PD-1 binding molecule, may be administered in further combination with a therapeutically or prophylactically effective amount of one or more therapeutic agents or chemotherapeutic agents known to those skilled in the art for the treatment and/or prevention of cancer, in particular a B7-H3-expressing cancer. Therapeutic agents and chemotherapeutic agents commonly used in the treatment of B7-H3-expressing cancers include, but are not limited to platinum-based chemotherapeutics (particularly, carboplatin, oxaliplatin, and carboplatin), taxanes (particularly, docetaxel and paclitaxel), hormonal therapies (particularly, abiraterone and enzalutamide), anthracyclines (particularly, daunorubicin, doxorubicin, and epirubicin), capecitabine, cyclophosphamide, leucovorin, methotrexate, radium 223, sipuleucel-T, and 5-fluorouracil (5-FU).

As used herein, the term “combination” refers to the use of more than one therapeutic agent. The use of the term “combination” does not restrict the order in which therapeutic agents are administered to a subject (e.g., a human patient or other mammal) with a disorder, nor does it mean that the agents are administered at exactly the same time. The term combination means that a B7-H3-ADC, a PD-1 binding molecule of the present invention, and any other agent are administered to a human patient or other mammal in a sequence and within a time interval such that the combination of a B7-H3-ADC, the PD-1 binding molecule and the other agent provide an increased benefit than if they were administered otherwise. For example, each therapeutic agent (e.g., chemotherapy, radiation therapy, hormonal therapy or biological therapy) may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route, e.g., one by the oral route and one parenterally, etc. Exemplary dosing regimens for administering a B7-H3-ADC in combination with a PD-1 binding molecule to a subject in need thereof are provided herein.

### X. Methods of Administration and Dose

A molecule of the invention (e.g., a B7-H3-ADC and/or a PD-1 binding molecule) can be administered by a variety of methods to a subject, e.g., a subject in need thereof, for example a human patient. For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection.

The molecules (e.g., a B7-H3-ADC, and/or a PD-1 binding molecule) can be administered as a flat dose (e.g., 375 mg) or as a weight-based dose (e.g., 3.5 mg/kg). The dose can also be selected to reduce or avoid production of antibodies against the administered molecules. Dosage regimens are adjusted to provide the desired response, e.g., a therapeutic response or a combinatorial therapeutic effect. Generally, doses of a B7-H3-ADC and the PD-1 binding molecule (and optionally a further agent) can be used in order to provide a subject with the agent in bioavailable quantities. As used herein, the term “dose” refers to a specified amount of medication taken at one time. The term “dosage” refers to the administering of a specific amount, number, and frequency of doses over a specified period of time; the term dosage thus includes chronological features, such as duration and periodicity.

The term “flat dose,” as used herein, refers to a dose that is independent of the weight of the patient, and includes physically discrete units of a molecule (e.g., a B7-H3-ADC or a PD-1 binding molecule) that are suited as a unitary dose for the subjects to be treated; wherein each unit contains a predetermined quantity of a B7-H3-ADC, and/or PD-1 binding molecule (calculated to produce a desired therapeutic effect) in association with a pharmaceutical carrier, and optionally, in association with a further agent. Single or multiple flat doses may be given. The term “weight-based dose” as used herein, refers to a discrete amount of a molecule to be administered per a unit of patient weight, for example milligrams of drug per kilograms of a subject's body weight (mg/kg body weight, abbreviated herein as “mg/kg”). The calculated dose will be administered based on the subject's body weight at baseline. Typically, a significant (≥10%) change in body weight from baseline or established plateau weight will generally prompt recalculation of dose. Single or multiple dosages may be given. Compositions comprising a B7-H3-ADC and/or a PD-1 binding molecule may be administered to a subject in need thereof via infusion.

In certain embodiments, a B7-H3-ADC is administered to a subject in need thereof at a weight-based dose of about 0.5 mg/kg to about 2 mg/kg, about 2 mg/kg to about 3 mg/kg, about 2 mg/kg to about 2.25 mg/kg, about 2.25 mg/kg to about 2.5 mg/kg, about 2.5 mg/kg to about 2.75 mg/kg, about 2.75 mg/kg to about 3 mg/kg, about 3 mg/kg to about 4 mg/kg, about 3 mg/kg to about 3.25 mg/kg, about 3.25 mg/kg to about 3.5 mg/kg, about 3.5 mg/kg to about 3.75 mg/kg, about 3.75 mg/kg to about 4 mg/kg, about 4 mg/kg to about 5 mg/kg, about 4 mg/kg to about 4.25 mg/kg, about 4.25 mg/kg to about 4.5 mg/kg, about 4.5 mg/kg to about 4.75 mg/kg, or about 5 mg/kg. In specific embodiments, B7-H3-ADC is administered to a subject in need thereof at a weight-based dose of about 0.5 mg/kg, about 1 mg/kg, about 1.5 mg/kg, about 2 mg/kg, about 2.25 mg/kg, about 2.5 mg/kg, about 2.75 mg/kg, about 3 mg/kg, about 3.25 mg/kg, about 3.5 mg/kg, about 3.75 mg/kg, about 4 mg/kg, about 4.25 mg/kg, about 4.5 mg/kg, about 4.75 mg/kg or about 5 mg/kg. In certain embodiments, a B7-H3-ADC is to be administered once about every 2 weeks, once about every 3 weeks, about once every 4 weeks, or more or less often.

In certain embodiments, the PD-1 binding molecule is hPD-1 mAb-A and is administered to a subject in need thereof at a flat dose of from about 200 mg to about 800 mg. In specific embodiments, hPD-1 mAb-A is administered to a subject in need thereof at a flat dose of about 200 mg, about 200 mg, about 275 mg, about 300 mg, about 350 mg, about 375 mg, about 400 mg, about 450 mg, about 475 mg, about 500 mg, about 550 mg, about 575 mg, about 600 mg, about 650 mg, about 675 mg, about 700 mg, about 750 mg, about 775 mg, or about 800 mg. In specific embodiments, hPD-1 mAb-A is administered to a subject in need thereof at a weight-based dose of from about 1 mg/kg to about 10 mg/kg. In specific embodiments, hPD-1 mAb-A is administered to a subject in need thereof at a weight-based dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg. In certain embodiments, the hPD-1 mAb-A is to be administered once about every 2 weeks, once about every 3 weeks, about once every 4 weeks, or more or less often.

In certain embodiments, the PD-1 binding molecule is pembrolizumab and is administered to a subject in need thereof at a flat dose of about 200 mg. In certain embodiments, nivolumab is administered to a subject in need thereof at a flat dose of about 240 mg or about 480 mg. In certain embodiments, nivolumab is administered to a subject in need thereof at a weight-based dose of about 3 mg/kg. In certain embodiments, pembrolizumab or nivolumab is to be administered once about every 2 weeks, once about every 3 weeks, about once every 4 weeks, or more or less often.

In specific embodiments, the PD-1 binding molecule is PD-1 X LAG-3 BD and is administered to a subject in need thereof at a flat dose of from about 120 mg to about 800 mg. In certain embodiments, the PD-1 X LAG-3 BD is administered to a subject in need thereof at a flat dose of about 120 mg, about 300 mg, about 400 mg, about 600 mg, or about 800 mg. In specific embodiments, the PD-1 X LAG-3 BD is administered to a subject in need thereof at a flat dose of about 300 mg. In another specific embodiment, the PD-1 X LAG-3 BD is administered to a subject in need thereof at a flat dose of about 600 mg. In another specific embodiment, a PD-1 X LAG-3 BD is administered to a subject in need thereof at a flat dose of about 800 mg. In certain embodiments, the PD-1 X LAG-3 BD is to be administered once about every 2 weeks, once about every 3 weeks, about once every 4 weeks, or more or less often.

With respect to flat doses or flat dosages, the term “about” is intended to denote a range that is ±10% of a recited dose, such that for example, a dose of about 600 mg will be between 540 mg and 660 mg. With respect to weight-based doses, the term “about” is intended to denote a range that is ±10% of a recited dose, such that for example, a dose of about 10 mg/kg will be between 0.9 mg/kg and 10.1 mg/kg.

The terms “dosing interval” and “dosing intervals” as used herein, refer to the time interval between doses, which can be regular or intermittent. A dosage of a molecule (e.g., a dose of a B7-H3-ADC and/or a dose of a PD-1 binding molecule) can be administered at a periodic dosing intervals over a period of time sufficient to encompass at least 2 doses, at least 4 doses, at least 6 doses, at least 12 doses, or at least 24 doses (a course of treatment). For example, a dosage may be administered e.g., once or twice daily, or about one to four times per week, or particularly once every week (“Q1W”), once every two weeks (“Q2W”), once every three weeks (“Q3W”), once every four weeks (“Q4W”), etc. Such periodic administration may continue for a period of time e.g., for between about 1 to 52 weeks, or for more than 52 weeks. Such course of treatment may be divided into increments, each referred to herein as a “cycle,” of e.g., between 2 to 8 weeks, between about 3 to 7 weeks, particularly about 4 weeks, or about 6 weeks, or about 8 weeks, during which a set number of doses are administered. The dose and/or the frequency of administration may be the same or different during each cycle. Factors that may influence the dosage and timing required to effectively treat a subject, include, e.g., the severity of the disease or disorder, formulation, route of delivery, previous treatments, the general health and/or age of the subject, and the presence of other diseases in the subject. Moreover, treatment of a subject with a therapeutically effective amount of a compound can include a single treatment or, can include a series of treatments.

A “dosing regimen” is a dosage administration in which a patient is administered a predetermined dose (or set of such predetermined doses) at a predetermined frequency (or set of such frequencies) for a predetermined periodicity (or periodicities). An exemplary dosing regimen comprises administration of a B7-H3-ADC of the present invention at a weight-based dose of about 0.5 mg/kg to about 5 mg/kg once every 3 weeks. Another exemplary dosing regimen comprises administration of a B7-H3-ADC at a weight-based dose of about 3 mg/kg to about 5 mg/kg once every 3 weeks. Another exemplary dosing regimen comprises administration of a B7-H3-ADC at a weight-based dose of about 3 mg/kg to about 4 mg/kg once every 3 weeks. Another exemplary dosing regimen comprises administration of a B7-H3-ADC at a weight-based dose of about 4 mg/kg to about 5 mg/kg once every 3 weeks. Another exemplary dosing regimen comprises administration of a B7-H3-ADC at a weight-based dose of about 1 mg/kg once every 3 weeks. Another exemplary dosing regimen comprises administration of a B7-H3-ADC at a weight-based dose of about 2 mg/kg once every 3 weeks. Another exemplary dosing regimen comprises administration of a B7-H3-ADC at a weight-based dose of about 3 mg/kg once every 3 weeks. Another exemplary dosing regimen comprises administration of a B7-H3-ADC at a weight-based dose of about 3.25 mg/kg once every 3 weeks. Another exemplary dosing regimen comprises administration of a B7-H3-ADC at a weight-based dose of about 3.5 mg/kg once every 3 weeks. Another exemplary dosing regimen comprises administration of a B7-H3-ADC at a weight-based dose of about 3.75 mg/kg once every 3 weeks. Another exemplary dosing regimen comprises administration of a B7-H3-ADC at a weight-based dose of about 4 mg/kg once every 3 weeks. Another exemplary dosing regimen comprises administration of a B7-H3-ADC at a weight-based dose of about 4.25 mg/kg once every 3 weeks. Another exemplary dosing regimen comprises administration of a B7-H3-ADC at a weight-based dose of about 4.5 mg/kg once every 3 weeks. Another exemplary dosing regimen comprises administration of a B7-H3-ADC at a weight-based dose of about 4.75 mg/kg once every 3 weeks. Another exemplary dosing regimen comprises administration of a B7-H3-ADC at a weight-based dose of about 5 mg/kg once every 3 weeks.

Another exemplary dosing regimen comprises administration of a B7-H3-ADC of the present invention at a weight-based dose of about 0.5 mg/kg to about 5 mg/kg once every 4 weeks. Another exemplary dosing regimen comprises administration of a B7-H3-ADC at a weight-based dose of about 3 mg/kg to about 5 mg/kg once every 4 weeks. Another exemplary dosing regimen comprises administration of a B7-H3-ADC at a weight-based dose of about 3 mg/kg to about 4 mg/kg once every 4 weeks. Another exemplary dosing regimen comprises administration of a B7-H3-ADC at a weight-based dose of about 4 mg/kg to about 5 mg/kg once every 4 weeks. Another exemplary dosing regimen comprises administration of a B7-H3-ADC at a weight-based dose of about 1 mg/kg once every 4 weeks. Another exemplary dosing regimen comprises administration of a B7-H3-ADC at a weight-based dose of about 2 mg/kg once every 4 weeks. Another exemplary dosing regimen comprises administration of a B7-H3-ADC at a weight-based dose of about 3 mg/kg once every 4 weeks. Another exemplary dosing regimen comprises administration of a B7-H3-ADC at a weight-based dose of about 3.25 mg/kg once every 4 weeks. Another exemplary dosing regimen comprises administration of a B7-H3-ADC at a weight-based dose of about 3.5 mg/kg once every 4 weeks. Another exemplary dosing regimen comprises administration of a B7-H3-ADC at a weight-based dose of about 3.75 mg/kg once every 4 weeks. Another exemplary dosing regimen comprises administration of a B7-H3-ADC at a weight-based dose of about 4 mg/kg once every 4 weeks. Another exemplary dosing regimen comprises administration of a B7-H3-ADC at a weight-based dose of about 4.25 mg/kg once every 4 weeks. Another exemplary dosing regimen comprises administration of a B7-H3-ADC at a weight-based dose of about 4.5 mg/kg once every 4 weeks. Another exemplary dosing regimen comprises administration of a B7-H3-ADC at a weight-based dose of about 4.75 mg/kg once every 4 weeks. Another exemplary dosing regimen comprises administration of a B7-H3-ADC at a weight-based dose of about 5 mg/kg once every 4 weeks.

Another exemplary dosing regimen comprises administration of a B7-H3-ADC of the present invention at a weight-based dose of about 0.5 mg/kg to about 5 mg/kg once every 3 weeks and a PD-1 binding molecule of the present invention at a flat dose of about 120 mg to about 800 mg administered once every 2 weeks, once every 3 weeks, or once every 4 weeks. Another exemplary dosing regimen comprises administration of a B7-H3-ADC at a weight-based dose of about 0.5 mg/kg to about 5 mg/kg once every 3 weeks and hPD-1 mAb-A at a flat dose of about 375 mg to about 500 mg once every 3 weeks. Another exemplary dosing regimen comprises administration of a B7-H3-ADC at a weight-based dose of about 3 mg/kg to about 5 mg/kg once every 3 weeks and hPD-1 mAb-A at a flat dose of about 375 mg to about 500 mg once every 3 weeks. Another exemplary dosing regimen comprises administration of a B7-H3-ADC at a weight-based dose of about 3 mg/kg to about 4 mg/kg once every 3 weeks and hPD-1 mAb-A at a flat dose of about 375 mg to about 500 mg once every 3 weeks. Another exemplary dosing regimen comprises administration of a B7-H3-ADC at a weight-based dose of about 4 mg/kg to about 5 mg/kg once every 3 weeks and hPD-1 mAb-A at a flat dose of about 375 mg to about 500 mg once every 3 weeks.

Another exemplary dosing regimen comprises administration of a B7-H3-ADC at a weight-based dose of about 3 mg/kg to about 4 mg/kg once every 3 weeks and hPD-1 mAb-A at a flat dose of about 375 mg once every 3 weeks. Another exemplary dosing regimen comprises administration of a B7-H3-ADC at a weight-based dose of about 4 mg/kg to about 5 mg/kg once every 3 weeks and hPD-1 mAb-A at a flat dose of about 375 mg once every 3 weeks. Another exemplary dosing regimen comprises administration of a B7-H3-ADC at a weight-based dose of about 2 mg/kg once every 3 weeks and hPD-1 mAb-A at a flat dose of about 375 mg every 3 weeks. Another exemplary dosing regimen comprises administration of a B7-H3-ADC at a weight-based dose of about 3 mg/kg once every 3 weeks and hPD-1 mAb-A at a flat dose of about 375 mg every 3 weeks. Another exemplary dosing regimen comprises administration of a B7-H3-ADC at a weight-based dose of about 3.25 mg/kg once every 3 weeks and hPD-1 mAb-A at a flat dose of about 375 mg every 3 weeks. Another exemplary dosing regimen comprises administration of a B7-H3-ADC at a weight-based dose of about 3.5 mg/kg once every 3 weeks and hPD-1 mAb-A at a flat dose of about 375 mg every 3 weeks. Another exemplary dosing regimen comprises administration of a B7-H3-ADC at a weight-based dose of about 3.75 mg/kg once every 3 weeks and hPD-1 mAb-A at a flat dose of about 375 mg every 3 weeks. Another exemplary dosing regimen comprises administration of a B7-H3-ADC at a weight-based dose of about 4 mg/kg once every 3 weeks and hPD-1 mAb-A at a flat dose of about 375 mg every 3 weeks. Another exemplary dosing regimen comprises administration of a B7-H3-ADC at a weight-based dose of about 4.25 mg/kg once every 3 weeks and hPD-1 mAb-A at a flat dose of about 375 mg every 3 weeks. Another exemplary dosing regimen comprises administration of a B7-H3-ADC at a weight-based dose of about 4.5 mg/kg once every 3 weeks and hPD-1 mAb-A at a flat dose of about 375 mg every 3 weeks. Another exemplary dosing regimen comprises administration of a B7-H3-ADC at a weight-based dose of about 4.75 mg/kg once every 3 weeks and hPD-1 mAb-A at a flat dose of about 375 mg every 3 weeks. Another exemplary dosing regimen comprises administration of a B7-H3-ADC at a weight-based dose of about 5 mg/kg once every 3 weeks and hPD-1 mAb-A at a flat dose of about 375 mg every 3 weeks.

Another exemplary dosing regimen comprises administration of a B7-H3-ADC at a weight-based dose of about 3 mg/kg to about 4 mg/kg once every 3 weeks and hPD-1 mAb-A at a flat dose of about 500 mg once every 3 weeks. Another exemplary dosing regimen comprises administration of a B7-H3-ADC at a weight-based dose of about 4 mg/kg to about 5 mg/kg once every 3 weeks and hPD-1 mAb-A at a flat dose of about 500 mg once every 3 weeks. Another exemplary dosing regimen comprises administration of a B7-H3-ADC at a weight-based dose of about 2 mg/kg once every 3 weeks and hPD-1 mAb-A at a flat dose of about 500 mg every 3 weeks. Another exemplary dosing regimen comprises administration of a B7-H3-ADC at a weight-based dose of about 3 mg/kg once every 3 weeks and hPD-1 mAb-A at a flat dose of about 500 mg every 3 weeks. Another exemplary dosing regimen comprises administration of a B7-H3-ADC at a weight-based dose of about 3.25 mg/kg once every 3 weeks and hPD-1 mAb-A at a flat dose of about 500 mg every 3 weeks. Another exemplary dosing regimen comprises administration of a B7-H3-ADC at a weight-based dose of about 3.5 mg/kg once every 3 weeks and hPD-1 mAb-A at a flat dose of about 500 mg every 3 weeks. Another exemplary dosing regimen comprises administration of a B7-H3-ADC at a weight-based dose of about 3.75 mg/kg once every 3 weeks and hPD-1 mAb-A at a flat dose of about 500 mg every 3 weeks. Another exemplary dosing regimen comprises administration of a B7-H3-ADC at a weight-based dose of about 4 mg/kg once every 3 weeks and hPD-1 mAb-A at a flat dose of about 500 mg every 3 weeks. Another exemplary dosing regimen comprises administration of a B7-H3-ADC at a weight-based dose of about 4.25 mg/kg once every 3 weeks and hPD-1 mAb-A at a flat dose of about 500 mg every 3 weeks. Another exemplary dosing regimen comprises administration of a B7-H3-ADC at a weight-based dose of about 4.5 mg/kg once every 3 weeks and hPD-1 mAb-A at a flat dose of about 500 mg every 3 weeks. Another exemplary dosing regimen comprises administration of a B7-H3-ADC at a weight-based dose of about 4.75 mg/kg once every 3 weeks and hPD-1 mAb-A at a flat dose of about 500 mg every 3 weeks. Another exemplary dosing regimen comprises administration of a B7-H3-ADC at a weight-based dose of about 5 mg/kg once every 3 weeks and hPD-1 mAb-A at a flat dose of about 500 mg every 3 weeks.

Another exemplary dosing regimen comprises administration of a B7-H3-ADC at a weight-based dose of about 0.5 mg/kg to about 5 mg/kg once every 3 weeks and hPD-1 mAb-A at a flat dose of about 375 mg to about 500 mg once every 4 weeks. Another exemplary dosing regimen comprises administration of a B7-H3-ADC at a weight-based dose of about 3 mg/kg to about 4 mg/kg once every 3 weeks and hPD-1 mAb-A at a flat dose of about 375 mg to about 500 mg once every 4 weeks. Another exemplary dosing regimen comprises administration of a B7-H3-ADC at a weight-based dose of about 4 mg/kg to about 5 mg/kg once every 3 weeks and hPD-1 mAb-A at a flat dose of about 375 mg to about 500 mg once every 4 weeks.

Another exemplary dosing regimen comprises administration of a B7-H3-ADC at a weight-based dose of about 3 mg/kg to about 4 mg/kg once every 3 weeks and hPD-1 mAb-A at a flat dose of about 375 mg once every 4 weeks. Another exemplary dosing regimen comprises administration of a B7-H3-ADC at a weight-based dose of about 4 mg/kg to about 5 mg/kg once every 3 weeks and hPD-1 mAb-A at a flat dose of about 375 mg once every 4 weeks. Another exemplary dosing regimen comprises administration of a B7-H3-ADC at a weight-based dose of about 2 mg/kg once every 3 weeks and hPD-1 mAb-A at a flat dose of about 375 mg every 4 weeks. Another exemplary dosing regimen comprises administration of a B7-H3-ADC at a weight-based dose of about 3 mg/kg once every 3 weeks and hPD-1 mAb-A at a flat dose of about 375 mg every 4 weeks. Another exemplary dosing regimen comprises administration of a B7-H3-ADC at a weight-based dose of about 3.25 mg/kg once every 3 weeks and hPD-1 mAb-A at a flat dose of about 375 mg every 4 weeks. Another exemplary dosing regimen comprises administration of a B7-H3-ADC at a weight-based dose of about 3.5 mg/kg once every 3 weeks and hPD-1 mAb-A at a flat dose of about 375 mg every 4 weeks. Another exemplary dosing regimen comprises administration of a B7-H3-ADC at a weight-based dose of about 3.75 mg/kg once every 3 weeks and hPD-1 mAb-A at a flat dose of about 375 mg every 4 weeks. Another exemplary dosing regimen comprises administration of a B7-H3-ADC at a weight-based dose of about 4 mg/kg once every 3 weeks and hPD-1 mAb-A at a flat dose of about 375 mg every 4 weeks. Another exemplary dosing regimen comprises administration of a B7-H3-ADC at a weight-based dose of about 4.25 mg/kg once every 3 weeks and hPD-1 mAb-A at a flat dose of about 375 mg every 4 weeks. Another exemplary dosing regimen comprises administration of a B7-H3-ADC at a weight-based dose of about 4.5 mg/kg once every 3 weeks and hPD-1 mAb-A at a flat dose of about 375 mg every 4 weeks. Another exemplary dosing regimen comprises administration of a B7-H3-ADC at a weight-based dose of about 4.75 mg/kg once every 3 weeks and hPD-1 mAb-A at a flat dose of about 375 mg every 4 weeks. Another exemplary dosing regimen comprises administration of a B7-H3-ADC at a weight-based dose of about 5 mg/kg once every 3 weeks and hPD-1 mAb-A at a flat dose of about 375 mg every 4 weeks.

Another exemplary dosing regimen comprises administration of a B7-H3-ADC at a weight-based dose of about 3 mg/kg to about 4 mg/kg once every 3 weeks and hPD-1 mAb-A at a flat dose of about 500 mg once every 4 weeks. Another exemplary dosing regimen comprises administration of a B7-H3-ADC at a weight-based dose of about 4 mg/kg to about 5 mg/kg once every 3 weeks and hPD-1 mAb-A at a flat dose of about 500 mg once every 4 weeks. Another exemplary dosing regimen comprises administration of a B7-H3-ADC at a weight-based dose of about 2 mg/kg once every 3 weeks and hPD-1 mAb-A at a flat dose of about 500 mg every 4 weeks. Another exemplary dosing regimen comprises administration of a B7-H3-ADC at a weight-based dose of about 3 mg/kg once every 3 weeks and hPD-1 mAb-A at a flat dose of about 500 mg every 4 weeks. Another exemplary dosing regimen comprises administration of a B7-H3-ADC at a weight-based dose of about 3.25 mg/kg once every 3 weeks and hPD-1 mAb-A at a flat dose of about 500 mg every 4 weeks. Another exemplary dosing regimen comprises administration of a B7-H3-ADC at a weight-based dose of about 3.5 mg/kg once every 3 weeks and hPD-1 mAb-A at a flat dose of about 500 mg every 4 weeks. Another exemplary dosing regimen comprises administration of a B7-H3-ADC at a weight-based dose of about 3.75 mg/kg once every 3 weeks and hPD-1 mAb-A at a flat dose of about 500 mg every 4 weeks. Another exemplary dosing regimen comprises administration of a B7-H3-ADC at a weight-based dose of about 4 mg/kg once every 3 weeks and hPD-1 mAb-A at a flat dose of about 500 mg every 4 weeks. Another exemplary dosing regimen comprises administration of a B7-H3-ADC at a weight-based dose of about 4.25 mg/kg once every 3 weeks and hPD-1 mAb-A at a flat dose of about 500 mg every 4 weeks. Another exemplary dosing regimen comprises administration of a B7-H3-ADC at a weight-based dose of about 4.5 mg/kg once every 3 weeks and hPD-1 mAb-A at a flat dose of about 500 mg every 4 weeks. Another exemplary dosing regimen comprises administration of a B7-H3-ADC at a weight-based dose of about 4.75 mg/kg once every 3 weeks and hPD-1 mAb-A at a flat dose of about 500 mg every 4 weeks. Another exemplary dosing regimen comprises administration of a B7-H3-ADC at a weight-based dose of about 5 mg/kg once every 3 weeks and hPD-1 mAb-A at a flat dose of about 500 mg every 4 weeks.

Another exemplary dosing regimen comprises administration of a B7-H3-ADC at a weight-based dose of about 0.5 mg/kg to about 5 mg/kg once every 4 weeks and hPD-1 mAb-A at a flat dose of about 375 mg to about 500 mg once every 3 weeks. Another exemplary dosing regimen comprises administration of a B7-H3-ADC at a weight-based dose of about 3 mg/kg to about 5 mg/kg once every 4 weeks and hPD-1 mAb-A at a flat dose of about 375 mg to about 500 mg once every 3 weeks. Another exemplary dosing regimen comprises administration of a B7-H3-ADC at a weight-based dose of about 3 mg/kg to about 4 mg/kg once every 4 weeks and hPD-1 mAb-A at a flat dose of about 375 mg to about 500 mg once every 3 weeks. Another exemplary dosing regimen comprises administration of a B7-H3-ADC at a weight-based dose of about 4 mg/kg to about 5 mg/kg once every 4 weeks and hPD-1 mAb-A at a flat dose of about 375 mg to about 500 mg once every 3 weeks.

Another exemplary dosing regimen comprises administration of a B7-H3-ADC at a weight-based dose of about 3 mg/kg to about 4 mg/kg once every 4 weeks and hPD-1 mAb-A at a flat dose of about 375 mg once every 3 weeks. Another exemplary dosing regimen comprises administration of a B7-H3-ADC at a weight-based dose of about 4 mg/kg to about 5 mg/kg once every 4 weeks and hPD-1 mAb-A at a flat dose of about 375 mg once every 3 weeks. Another exemplary dosing regimen comprises administration of a B7-H3-ADC at a weight-based dose of about 2 mg/kg once every 4 weeks and hPD-1 mAb-A at a flat dose of about 375 mg every 3 weeks. Another exemplary dosing regimen comprises administration of a B7-H3-ADC at a weight-based dose of about 3 mg/kg once every 4 weeks and hPD-1 mAb-A at a flat dose of about 375 mg every 3 weeks. Another exemplary dosing regimen comprises administration of a B7-H3-ADC at a weight-based dose of about 3.25 mg/kg once every 4 weeks and hPD-1 mAb-A at a flat dose of about 375 mg every 3 weeks. Another exemplary dosing regimen comprises administration of a B7-H3-ADC at a weight-based dose of about 3.5 mg/kg once every 4 weeks and hPD-1 mAb-A at a flat dose of about 375 mg every 3 weeks. Another exemplary dosing regimen comprises administration of a B7-H3-ADC at a weight-based dose of about 3.75 mg/kg once every 4 weeks and hPD-1 mAb-A at a flat dose of about 375 mg every 3 weeks. Another exemplary dosing regimen comprises administration of a B7-H3-ADC at a weight-based dose of about 4 mg/kg once every 3 weeks and hPD-1 mAb-A at a flat dose of about 375 mg every 3 weeks. Another exemplary dosing regimen comprises administration of a B7-H3-ADC at a weight-based dose of about 4.25 mg/kg once every 4 weeks and hPD-1 mAb-A at a flat dose of about 375 mg every 3 weeks. Another exemplary dosing regimen comprises administration of a B7-H3-ADC at a weight-based dose of about 4.5 mg/kg once every 4 weeks and hPD-1 mAb-A at a flat dose of about 375 mg every 3 weeks. Another exemplary dosing regimen comprises administration of a B7-H3-ADC at a weight-based dose of about 4.75 mg/kg once every 4 weeks and hPD-1 mAb-A at a flat dose of about 375 mg every 3 weeks. Another exemplary dosing regimen comprises administration of a B7-H3-ADC at a weight-based dose of about 5 mg/kg once every 4 weeks and hPD-1 mAb-A at a flat dose of about 375 mg every 3 weeks.

Another exemplary dosing regimen comprises administration of a B7-H3-ADC at a weight-based dose of about 3 mg/kg to about 4 mg/kg once every 4 weeks and hPD-1 mAb-A at a flat dose of about 500 mg once every 3 weeks. Another exemplary dosing regimen comprises administration of a B7-H3-ADC at a weight-based dose of about 4 mg/kg to about 5 mg/kg once every 4 weeks and hPD-1 mAb-A at a flat dose of about 500 mg once every 3 weeks. Another exemplary dosing regimen comprises administration of a B7-H3-ADC at a weight-based dose of about 2 mg/kg once every 4 weeks and hPD-1 mAb-A at a flat dose of about 500 mg every 3 weeks. Another exemplary dosing regimen comprises administration of a B7-H3-ADC at a weight-based dose of about 3 mg/kg once every 4 weeks and hPD-1 mAb-A at a flat dose of about 500 mg every 3 weeks. Another exemplary dosing regimen comprises administration of a B7-H3-ADC at a weight-based dose of about 3.25 mg/kg once every 4 weeks and hPD-1 mAb-A at a flat dose of about 500 mg every 3 weeks. Another exemplary dosing regimen comprises administration of a B7-H3-ADC at a weight-based dose of about 3.5 mg/kg once every 4 weeks and hPD-1 mAb-A at a flat dose of about 500 mg every 3 weeks. Another exemplary dosing regimen comprises administration of a B7-H3-ADC at a weight-based dose of about 3.75 mg/kg once every 4 weeks and hPD-1 mAb-A at a flat dose of about 500 mg every 3 weeks. Another exemplary dosing regimen comprises administration of a B7-H3-ADC at a weight-based dose of about 4 mg/kg once every 4 weeks and hPD-1 mAb-A at a flat dose of about 500 mg every 3 weeks. Another exemplary dosing regimen comprises administration of a B7-H3-ADC at a weight-based dose of about 4.25 mg/kg once every 4 weeks and hPD-1 mAb-A at a flat dose of about 500 mg every 3 weeks. Another exemplary dosing regimen comprises administration of a B7-H3-ADC at a weight-based dose of about 4.5 mg/kg once every 4 weeks and hPD-1 mAb-A at a flat dose of about 500 mg every 3 weeks. Another exemplary dosing regimen comprises administration of a B7-H3-ADC at a weight-based dose of about 4.75 mg/kg once every 4 weeks and hPD-1 mAb-A at a flat dose of about 500 mg every 3 weeks. Another exemplary dosing regimen comprises administration of a B7-H3-ADC at a weight-based dose of about 5 mg/kg once every 4 weeks and hPD-1 mAb-A at a flat dose of about 500 mg every 3 weeks.

Another exemplary dosing regimen comprises administration of a B7-H3-ADC of the present invention at a weight-based dose of about 0.5 mg/kg to about 5 mg/kg once every 3 weeks and hPD-1 mAb-A of the present invention at a weight-based dose of about 1 mg/kg to about 10 mg/kg, administered once every 2 weeks, once every 3 weeks, or once every 4 weeks. Another exemplary dosing regimen comprises administration of a B7-H3-ADC at a weight-based dose of about 0.5 mg/kg to about 5 mg/kg once every 3 weeks and hPD-1 mAb-A at a weight-based dose of about 1 mg/kg to about 10 mg/kg once every 2 weeks. Another exemplary dosing regimen comprises administration of a B7-H3-ADC at a weight-based dose of about 3 mg/kg to about 4 mg/kg once every 3 weeks and hPD-1 mAb-A at a weight-based dose of about 1 mg/kg to about 10 mg/kg once every 2 weeks. Another exemplary dosing regimen comprises administration of a B7-H3-ADC at a weight-based dose of about 4 mg/kg to about 5 mg/kg once every 3 weeks and hPD-1 mAb-A at a weight-based dose of about 1 mg/kg to about 10 mg/kg once every 2 weeks. Another exemplary dosing regimen comprises administration of a B7-H3-ADC at a weight-based dose of about 0.5 mg/kg to about 5 mg/kg once every 3 weeks and hPD-1 mAb-A at a dose of about 1 mg/kg once every 2 weeks. Another exemplary dosing regimen comprises administration of a B7-H3-ADC at a weight-based dose of about 0.5 mg/kg to about 5 mg/kg once every 3 weeks and hPD-1 mAb-A at a dose of about 3 mg/kg once every 2 weeks. Another exemplary dosing regimen comprises administration of a B7-H3-ADC at a weight-based dose of about 0.5 mg/kg to about 5 mg/kg once every 3 weeks and hPD-1 mAb-A at weight-based dose of about 10 mg/kg once every 2 weeks. Another exemplary dosing regimen comprises administration of a B7-H3-ADC at a weight-based dose of about 3 mg/kg to about 4 mg/kg once every 3 weeks and hPD-1 mAb-A at dose of about 1 mg/kg once every 2 weeks. Another exemplary dosing regimen comprises administration of a B7-H3-ADC at a weight-based dose of about 3 mg/kg to about 4 mg/kg once every 3 weeks and hPD-1 mAb-A at a dose of about 3 mg/kg once every 2 weeks. Another exemplary dosing regimen comprises administration of a B7-H3-ADC at a weight-based dose of about 3 mg/kg to about 4 mg/kg once every 3 weeks and hPD-1 mAb-A at a dose of about 10 mg/kg once every 2 weeks. Another exemplary dosing regimen comprises administration of a B7-H3-ADC at a weight-based dose of about 4 mg/kg to about 5 mg/kg once every 3 weeks and hPD-1 mAb-A at dose of about 1 mg/kg once every 2 weeks. Another exemplary dosing regimen comprises administration of a B7-H3-ADC at a weight-based dose of about 4 mg/kg to about 5 mg/kg once every 3 weeks and hPD-1 mAb-A at a dose of about 3 mg/kg once every 2 weeks. Another exemplary dosing regimen comprises administration of a B7-H3-ADC at a weight-based dose of about 4 mg/kg to about 5 mg/kg once every 3 weeks and hPD-1 mAb-A at a dose of about 10 mg/kg once every 2 weeks.

Another exemplary dosing regimen comprises administration of a B7-H3-ADC at a weight-based dose of about 0.5 mg/kg to about 5 mg/kg once every 3 weeks and hPD-1 mAb-A at a weight-based dose of about 1 mg/kg to about 10 mg/kg once every 3 weeks. Another exemplary dosing regimen comprises administration of a B7-H3-ADC at a weight-based dose of about 3 mg/kg to about 4 mg/kg once every 3 weeks and hPD-1 mAb-A at a weight-based dose of about 1 mg/kg to about 10 mg/kg once every 3 weeks. Another exemplary dosing regimen comprises administration of a B7-H3-ADC at a weight-based dose of about 4 mg/kg to about 5 mg/kg once every 3 weeks and hPD-1 mAb-A at a weight-based dose of about 1 mg/kg to about 10 mg/kg once every 3 weeks. Another exemplary dosing regimen comprises administration of a B7-H3-ADC at a weight-based dose of about 0.5 mg/kg to about 5 mg/kg once every 3 weeks and hPD-1 mAb-A at dose of about 3 mg/kg to about 10 mg/kg once every 3 weeks. Another exemplary dosing regimen comprises administration of a B7-H3-ADC at a weight-based dose of about 3 mg/kg to about 4 mg/kg once every 3 weeks and hPD-1 mAb-A at a dose of about 3 mg/kg once every 3 weeks. Another exemplary dosing regimen comprises administration of a B7-H3-ADC at a weight-based dose of about 4 mg/kg to about 5 mg/kg once every 3 weeks and hPD-1 mAb-A at a dose of about 3 mg/kg once every 3 weeks.

Another exemplary dosing regimen comprises administration of a B7-H3-ADC at a weight-based dose of about 0.5 mg/kg to about 5 mg/kg once every 3 weeks and hPD-1 mAb-A at weight-based dose of about 1 mg/kg to about 10 mg/kg once every 4 weeks. Another exemplary dosing regimen comprises administration of a B7-H3-ADC at a weight-based dose of about 3 mg/kg to about 4 mg/kg once every 3 weeks and hPD-1 mAb-A at a weight-based dose of about 1 mg/kg to about 10 mg/kg once every 4 weeks. Another exemplary dosing regimen comprises administration of a B7-H3-ADC at a weight-based dose of about 4 mg/kg to about 5 mg/kg once every 3 weeks and hPD-1 mAb-A at a weight-based dose of about 1 mg/kg to about 10 mg/kg once every 4 weeks. Another exemplary dosing regimen comprises administration of a B7-H3-ADC at a weight-based dose of about 0.5 mg/kg to about 5 mg/kg once every 3 weeks and hPD-1 mAb-A at weight-based dose of about 3 mg/kg once every 4 weeks. Another exemplary dosing regimen comprises administration of a B7-H3-ADC at a weight-based dose of about 3 mg/kg to about 4 mg/kg once every 3 weeks and hPD-1 mAb-A at weight-based dose of about 3 mg/kg once every 4 weeks. Another exemplary dosing regimen comprises administration of a B7-H3-ADC at a weight-based dose of about 4 mg/kg to about 5 mg/kg once every 3 weeks and hPD-1 mAb-A at weight-based dose of about 3 mg/kg once every 4 weeks. Another exemplary dosing regimen comprises administration of a B7-H3-ADC at a weight-based dose of about 0.5 mg/kg to about 5 mg/kg once every 3 weeks and hPD-1 mAb-A at weight-based dose of about 10 mg/kg once every 4 weeks. Another exemplary dosing regimen comprises administration of a B7-H3-ADC at a weight-based dose of about 3 mg/kg to about 4 mg/kg once every 3 weeks and hPD-1 mAb-A at a dose of about 10 mg/kg once every 4 weeks. Another exemplary dosing regimen comprises administration of a B7-H3-ADC at a weight-based dose of about 4 mg/kg to about 5 mg/kg once every 3 weeks and hPD-1 mAb-A at a dose of about 10 mg/kg once every 4 weeks.

An exemplary dosing regimen comprises administration of a B7-H3-ADC of the present invention at a weight-based dose of about 0.5 mg/kg to about 5 mg/kg once every 3 weeks and pembrolizumab at a flat dose of about 200 mg once every 3 weeks. Another exemplary dosing regimen comprises administration of a B7-H3-ADC at a weight-based dose of about 3 mg/kg to about 5 mg/kg once every 3 weeks and pembrolizumab at a flat dose of about 200 mg once every 3 weeks. Another exemplary dosing regimen comprises administration of a B7-H3-ADC at a weight-based dose of about 3 mg/kg to about 4 mg/kg once every 3 weeks and pembrolizumab at a flat dose of about 200 mg once every 3 weeks. Another exemplary dosing regimen comprises administration of a B7-H3-ADC at a weight-based dose of about 4 mg/kg to about 5 mg/kg once every 3 weeks and pembrolizumab at a flat dose of about 200 mg once every 3 weeks. Another exemplary dosing regimen comprises administration of a B7-H3-ADC at a weight-based dose of about 2 mg/kg once every 3 weeks and pembrolizumab at a flat dose of about 200 mg once every 3 weeks. Another exemplary dosing regimen comprises administration of a B7-H3-ADC at a weight-based dose of about 3 mg/kg once every 3 weeks and pembrolizumab at a flat dose of about 200 mg once every 3 weeks. Another exemplary dosing regimen comprises administration of a B7-H3-ADC at a weight-based dose of about 3.25 mg/kg once every 3 weeks and pembrolizumab at a flat dose of about 200 mg once every 3 weeks. Another exemplary dosing regimen comprises administration of a B7-H3-ADC at a weight-based dose of about 3.5 mg/kg once every 3 weeks and pembrolizumab at a flat dose of about 200 mg once every 3 weeks. Another exemplary dosing regimen comprises administration of a B7-H3-ADC at a weight-based dose of about 3.75 mg/kg once every 3 weeks and pembrolizumab at a flat dose of about 200 mg once every 3 weeks. Another exemplary dosing regimen comprises administration of a B7-H3-ADC at a weight-based dose of about 4 mg/kg once every 3 weeks and pembrolizumab at a flat dose of about 200 mg once every 3 weeks. Another exemplary dosing regimen comprises administration of a B7-H3-ADC at a weight-based dose of about 4.25 mg/kg once every 3 weeks and pembrolizumab at a flat dose of about 200 mg once every 3 weeks. Another exemplary dosing regimen comprises administration of a B7-H3-ADC at a weight-based dose of about 4.5 mg/kg once every 3 weeks and pembrolizumab at a flat dose of about 200 mg once every 3 weeks. Another exemplary dosing regimen comprises administration of a B7-H3-ADC at a weight-based dose of about 4.75 mg/kg once every 3 weeks and pembrolizumab at a flat dose of about 200 mg once every 3 weeks. Another exemplary dosing regimen comprises administration of a B7-H3-ADC at a weight-based dose of about 5 mg/kg once every 3 weeks and pembrolizumab at a flat dose of about 200 mg once every 3 weeks.

An exemplary dosing regimen comprises administration of a B7-H3-ADC of the present invention at a weight-based dose of about 0.5 mg/kg to about 5 mg/kg once every 3 weeks and nivolumab at a flat dose of about 240 mg once every 2 weeks. Another exemplary dosing regimen comprises administration of a B7-H3-ADC at a weight-based dose of about 3 mg/kg to about 5 mg/kg once every 3 weeks and nivolumab at a flat dose of about 240 mg once every 2 weeks. Another exemplary dosing regimen comprises administration of a B7-H3-ADC at a weight-based dose of about 3 mg/kg to about 4 mg/kg once every 3 weeks and nivolumab at a flat dose of about 240 mg once every 2 weeks. Another exemplary dosing regimen comprises administration of a B7-H3-ADC at a weight-based dose of about 4 mg/kg to about 5 mg/kg once every 3 weeks and nivolumab at a flat dose of about 240 mg once every 2 weeks. Another exemplary dosing regimen comprises administration of a B7-H3-ADC at a weight-based dose of about 2 mg/kg once every 3 weeks and nivolumab at a flat dose of about 240 mg once every 2 weeks. Another exemplary dosing regimen comprises administration of a B7-H3-ADC at a weight-based dose of about 3 mg/kg once every 3 weeks and nivolumab at a flat dose of about 240 mg once every 2 weeks. Another exemplary dosing regimen comprises administration of a B7-H3-ADC at a weight-based dose of about 3.25 mg/kg once every 3 weeks and nivolumab at a flat dose of about 240 mg once every 2 weeks. Another exemplary dosing regimen comprises administration of a B7-H3-ADC at a weight-based dose of about 3.5 mg/kg once every 3 weeks and nivolumab at a flat dose of about 240 mg once every 2 weeks. Another exemplary dosing regimen comprises administration of a B7-H3-ADC at a weight-based dose of about 3.75 mg/kg once every 3 weeks and nivolumab at a flat dose of about 240 mg once every 2 weeks. Another exemplary dosing regimen comprises administration of a B7-H3-ADC at a weight-based dose of about 4 mg/kg once every 3 weeks and nivolumab at a flat dose of about 240 mg once every 2 weeks. Another exemplary dosing regimen comprises administration of a B7-H3-ADC at a weight-based dose of about 4.25 mg/kg once every 3 weeks and nivolumab at a flat dose of about 240 mg once every 2 weeks. Another exemplary dosing regimen comprises administration of a B7-H3-ADC at a weight-based dose of about 4.5 mg/kg once every 3 weeks and nivolumab at a flat dose of about 240 mg once every 2 weeks. Another exemplary dosing regimen comprises administration of a B7-H3-ADC at a weight-based dose of about 4.75 mg/kg once every 3 weeks and nivolumab at a flat dose of about 240 mg once every 2 weeks. Another exemplary dosing regimen comprises administration of a B7-H3-ADC at a weight-based dose of about 5 mg/kg once every 3 weeks and nivolumab at a flat dose of about 240 mg once every 2 weeks.

Another exemplary dosing regimen comprises administration of a B7-H3-ADC at a weight-based dose of about 0.5 mg/kg to about 5 mg/kg once every 3 weeks and nivolumab at a flat dose of about 480 mg once every 4 weeks. Another exemplary dosing regimen comprises administration of a B7-H3-ADC at a weight-based dose of about 3 mg/kg to about 5 mg/kg once every 3 weeks and nivolumab at a flat dose of about 480 mg once every 4 weeks. Another exemplary dosing regimen comprises administration of a B7-H3-ADC at a weight-based dose of about 3 mg/kg to about 4 mg/kg once every 3 weeks and nivolumab at a flat dose of about 480 mg once every 4 weeks. Another exemplary dosing regimen comprises administration of a B7-H3-ADC at a weight-based dose of about 4 mg/kg to about 5 mg/kg once every 3 weeks and nivolumab at a flat dose of about 480 mg once every 4 weeks. Another exemplary dosing regimen comprises administration of a B7-H3-ADC at a weight-based dose of about 2 mg/kg once every 3 weeks and nivolumab at a flat dose of about 480 mg once every 4 weeks. Another exemplary dosing regimen comprises administration of a B7-H3-ADC at a weight-based dose of about 3 mg/kg once every 3 weeks and nivolumab at a flat dose of about 480 mg once every 4 weeks. Another exemplary dosing regimen comprises administration of a B7-H3-ADC at a weight-based dose of about 3.25 mg/kg once every 3 weeks and nivolumab at a flat dose of about 480 mg once every 4 weeks. Another exemplary dosing regimen comprises administration of a B7-H3-ADC at a weight-based dose of about 3.5 mg/kg once every 3 weeks and nivolumab at a flat dose of about 480 mg once every 4 weeks. Another exemplary dosing regimen comprises administration of a B7-H3-ADC at a weight-based dose of about 3.75 mg/kg once every 3 weeks and nivolumab at a flat dose of about 480 mg once every 4 weeks. Another exemplary dosing regimen comprises administration of a B7-H3-ADC at a weight-based dose of about 4 mg/kg once every 3 weeks and nivolumab at a flat dose of about 480 mg once every 4 weeks. Another exemplary dosing regimen comprises administration of a B7-H3-ADC at a weight-based dose of about 4.25 mg/kg once every 3 weeks and nivolumab at a flat dose of about 480 mg once every 4 weeks. Another exemplary dosing regimen comprises administration of a B7-H3-ADC at a weight-based dose of about 4.5 mg/kg once every 3 weeks and nivolumab at a flat dose of about 480 mg once every 4 weeks. Another exemplary dosing regimen comprises administration of a B7-H3-ADC at a weight-based dose of about 4.75 mg/kg once every 3 weeks and nivolumab at a flat dose of about 480 mg once every 4 weeks. Another exemplary dosing regimen comprises administration of a B7-H3-ADC at a weight-based dose of about 5 mg/kg once every 3 weeks and nivolumab at a flat dose of about 480 mg once every 4 weeks.

Another exemplary dosing regimen comprises administration of a B7-H3-ADC at a weight-based dose of about 0.5 mg/kg to about 5 mg/kg once every 3 weeks and nivolumab at a weight-based dose of about 3 mg/kg once every 3 weeks. Another exemplary dosing regimen comprises administration of a B7-H3-ADC at a weight-based dose of about 3 mg/kg to about 5 mg/kg once every 3 weeks and nivolumab at a weight-based dose of about 3 mg/kg once every 3 weeks. Another exemplary dosing regimen comprises administration of a B7-H3-ADC at a weight-based dose of about 3 mg/kg to about 4 mg/kg once every 3 weeks and nivolumab at a weight-based dose of about 3 mg/kg once every 3 weeks. Another exemplary dosing regimen comprises administration of a B7-H3-ADC at a weight-based dose of about 4 mg/kg to about 5 mg/kg once every 3 weeks and nivolumab at a weight-based dose of about 3 mg/kg once every 3 weeks. Another exemplary dosing regimen comprises administration of a B7-H3-ADC at a weight-based dose of about 2 mg/kg once every 3 weeks and nivolumab at a weight-based dose of about 3 mg/kg once every 3 weeks. Another exemplary dosing regimen comprises administration of a B7-H3-ADC at a weight-based dose of about 3 mg/kg once every 3 weeks and nivolumab at a weight-based dose of about 3 mg/kg once every 3 weeks. Another exemplary dosing regimen comprises administration of a B7-H3-ADC at a weight-based dose of about 3.25 mg/kg once every 3 weeks and nivolumab at a weight-based dose of about 3 mg/kg once every 3 weeks. Another exemplary dosing regimen comprises administration of a B7-H3-ADC at a weight-based dose of about 3.5 mg/kg once every 3 weeks and nivolumab at a weight-based dose of about 3 mg/kg once every 3 weeks. Another exemplary dosing regimen comprises administration of a B7-H3-ADC at a weight-based dose of about 3.75 mg/kg once every 3 weeks and nivolumab at a weight-based dose of about 3 mg/kg once every 3 weeks. Another exemplary dosing regimen comprises administration of a B7-H3-ADC at a weight-based dose of about 4 mg/kg once every 3 weeks and nivolumab at a weight-based dose of about 3 mg/kg once every 3 weeks. Another exemplary dosing regimen comprises administration of a B7-H3-ADC at a weight-based dose of about 4.25 mg/kg once every 3 weeks and nivolumab at a weight-based dose of about 3 mg/kg once every 3 weeks. Another exemplary dosing regimen comprises administration of a B7-H3-ADC at a weight-based dose of about 4.5 mg/kg once every 3 weeks and nivolumab at a weight-based dose of about 3 mg/kg once every 3 weeks. Another exemplary dosing regimen comprises administration of a B7-H3-ADC at a weight-based dose of about 4.75 mg/kg once every 3 weeks and nivolumab at a weight-based dose of about 3 mg/kg once every 3 weeks. Another exemplary dosing regimen comprises administration of a B7-H3-ADC at a weight-based dose of about 5 mg/kg once every 3 weeks and nivolumab at a weight-based dose of about 3 mg/kg once every 3 weeks.

An exemplary dosing regimen comprises administration of a B7-H3-ADC of the present invention at a weight-based dose of about 0.5 mg/kg to about 5 mg/kg once every 3 weeks and PD-1 X LAG-3 BD at a flat dose of about 120 mg to about 800 mg once every 2 weeks. Another exemplary dosing regimen comprises administration of a B7-H3-ADC at a weight-based dose of about 3 mg/kg to about 5 mg/kg once every 3 weeks and PD-1 X LAG-3 BD at a flat dose of about 120 mg to about 800 mg once every 2 weeks. Another exemplary dosing regimen comprises administration of a B7-H3-ADC at a weight-based dose of about 3 mg/kg to about 4 mg/kg once every 3 weeks and PD-1 X LAG-3 BD at a flat dose of about 120 mg to about 800 mg once every 2 weeks. Another exemplary dosing regimen comprises administration of a B7-H3-ADC at a weight-based dose of about 4 mg/kg to about 5 mg/kg once every 3 weeks and PD-1 X LAG-3 BD at a flat dose of about 120 mg to about 800 mg once every 2 weeks. Another exemplary dosing regimen comprises administration of a B7-H3-ADC at a weight-based dose of about 2 mg/kg once every 3 weeks and PD-1 X LAG-3 BD at a flat dose of about 120 mg to about 800 mg once every 2 weeks. Another exemplary dosing regimen comprises administration of a B7-H3-ADC at a weight-based dose of about 3 mg/kg once every 3 weeks and PD-1 X LAG-3 BD at a flat dose of about 120 mg to about 800 mg once every 2 weeks. Another exemplary dosing regimen comprises administration of a B7-H3-ADC at a weight-based dose of about 3.25 mg/kg once every 3 weeks and PD-1 X LAG-3 BD at a flat dose of about 120 mg to about 800 mg once every 2 weeks. Another exemplary dosing regimen comprises administration of a B7-H3-ADC at a weight-based dose of about 3.5 mg/kg once every 3 weeks and PD-1 X LAG-3 BD at a flat dose of about 120 mg to about 800 mg once every 2 weeks. Another exemplary dosing regimen comprises administration of a B7-H3-ADC at a weight-based dose of about 3.75 mg/kg once every 3 weeks and PD-1 X LAG-3 BD at a flat dose of about 120 mg to about 800 mg once every 2 weeks. Another exemplary dosing regimen comprises administration of a B7-H3-ADC at a weight-based dose of about 4 mg/kg once every 3 weeks and PD-1 X LAG-3 BD at a flat dose of about 120 mg to about 800 mg once every 2 weeks. Another exemplary dosing regimen comprises administration of a B7-H3-ADC at a weight-based dose of about 4.25 mg/kg once every 3 weeks and PD-1 X LAG-3 BD at a flat dose of about 120 mg to about 800 mg once every 2 weeks. Another exemplary dosing regimen comprises administration of a B7-H3-ADC at a weight-based dose of about 4.5 mg/kg once every 3 weeks and PD-1 X LAG-3 BD at a flat dose of about 120 mg to about 800 mg once every 2 weeks. Another exemplary dosing regimen comprises administration of a B7-H3-ADC at a weight-based dose of about 4.75 mg/kg once every 3 weeks and PD-1 X LAG-3 BD at a flat dose of about 120 mg to about 800 mg once every 2 weeks. Another exemplary dosing regimen comprises administration of a B7-H3-ADC at a weight-based dose of about 5 mg/kg once every 3 weeks and PD-1 X LAG-3 BD at a flat dose of about 120 mg to about 800 mg once every 2 weeks.

Another exemplary dosing regimen comprises administration of a B7-H3-ADC at a weight-based dose of about 3 mg/kg to about 4 mg/kg once every 3 weeks and PD-1 X LAG-3 BD at a flat dose of about 300 mg once every 2 weeks. Another exemplary dosing regimen comprises administration of a B7-H3-ADC at a weight-based dose of about 4 mg/kg to about 5 mg/kg once every 3 weeks and PD-1 X LAG-3 BD at a flat dose of about 300 mg once every 2 weeks. Another exemplary dosing regimen comprises administration of a B7-H3-ADC at a weight-based dose of about 2 mg/kg once every 3 weeks and PD-1 X LAG-3 BD at a flat dose of about 300 mg once every 2 weeks. Another exemplary dosing regimen comprises administration of a B7-H3-ADC at a weight-based dose of about 3 mg/kg once every 3 weeks and PD-1 X LAG-3 BD at a flat dose of about 300 mg once every 2 weeks. Another exemplary dosing regimen comprises administration of a B7-H3-ADC at a weight-based dose of about 3.25 mg/kg once every 3 weeks and PD-1 X LAG-3 BD at a flat dose of about 300 mg once every 2 weeks. Another exemplary dosing regimen comprises administration of a B7-H3-ADC at a weight-based dose of about 3.5 mg/kg once every 3 weeks and PD-1 X LAG-3 BD at a flat dose of about 300 mg once every 2 weeks. Another exemplary dosing regimen comprises administration of a B7-H3-ADC at a weight-based dose of about 3.75 mg/kg once every 3 weeks and PD-1 X LAG-3 BD at a flat dose of about 300 mg once every 2 weeks. Another exemplary dosing regimen comprises administration of a B7-H3-ADC at a weight-based dose of about 4 mg/kg once every 3 weeks and PD-1 X LAG-3 BD at a flat dose of about 300 mg once every 2 weeks. Another exemplary dosing regimen comprises administration of a B7-H3-ADC at a weight-based dose of about 4.25 mg/kg once every 3 weeks and PD-1 X LAG-3 BD at a flat dose of about 300 mg once every 2 weeks. Another exemplary dosing regimen comprises administration of a B7-H3-ADC at a weight-based dose of about 4.5 mg/kg once every 3 weeks and PD-1 X LAG-3 BD at a flat dose of about 300 mg once every 2 weeks. Another exemplary dosing regimen comprises administration of a B7-H3-ADC at a weight-based dose of about 4.75 mg/kg once every 3 weeks and PD-1 X LAG-3 BD at a flat dose of about 300 mg once every 2 weeks. Another exemplary dosing regimen comprises administration of a B7-H3-ADC at a weight-based dose of about 5 mg/kg once every 3 weeks and PD-1 X LAG-3 BD at a flat dose of about 300 mg once every 2 weeks.

Another exemplary dosing regimen comprises administration of a B7-H3-ADC at a weight-based dose of about 3 mg/kg to about 4 mg/kg once every 3 weeks and PD-1 X LAG-3 BD at a flat dose of about 600 mg once every 2 weeks. Another exemplary dosing regimen comprises administration of a B7-H3-ADC at a weight-based dose of about 4 mg/kg to about 5 mg/kg once every 3 weeks and PD-1 X LAG-3 BD at a flat dose of about 600 mg once every 2 weeks. Another exemplary dosing regimen comprises administration of a B7-H3-ADC at a weight-based dose of about 2 mg/kg once every 3 weeks and PD-1 X LAG-3 BD at a flat dose of about 600 mg once every 2 weeks. Another exemplary dosing regimen comprises administration of a B7-H3-ADC at a weight-based dose of about 3 mg/kg once every 3 weeks and PD-1 X LAG-3 BD at a flat dose of about 600 mg once every 2 weeks. Another exemplary dosing regimen comprises administration of a B7-H3-ADC at a weight-based dose of about 3.25 mg/kg once every 3 weeks and PD-1 X LAG-3 BD at a flat dose of about 600 mg once every 2 weeks. Another exemplary dosing regimen comprises administration of a B7-H3-ADC at a weight-based dose of about 3.75 mg/kg once every 3 weeks and PD-1 X LAG-3 BD at a flat dose of about 600 mg once every 2 weeks. Another exemplary dosing regimen comprises administration of a B7-H3-ADC at a weight-based dose of about 4 mg/kg once every 3 weeks and PD-1 X LAG-3 BD at a flat dose of about 600 mg once every 2 weeks. Another exemplary dosing regimen comprises administration of a B7-H3-ADC at a weight-based dose of about 4.25 mg/kg once every 3 weeks and PD-1 X LAG-3 BD at a flat dose of about 600 mg once every 2 weeks. Another exemplary dosing regimen comprises administration of a B7-H3-ADC at a weight-based dose of about 4.5 mg/kg once every 3 weeks and PD-1 X LAG-3 BD at a flat dose of about 600 mg once every 2 weeks. Another exemplary dosing regimen comprises administration of a B7-H3-ADC at a weight-based dose of about 4.75 mg/kg once every 3 weeks and PD-1 X LAG-3 BD at a flat dose of about 600 mg once every 2 weeks. Another exemplary dosing regimen comprises administration of a B7-H3-ADC at a weight-based dose of about 5 mg/kg once every 3 weeks and PD-1 X LAG-3 BD at a flat dose of about 600 mg once every 2 weeks.

Another exemplary dosing regimen comprises administration of a B7-H3-ADC at a weight-based dose of about 0.5 mg/kg to about 5 mg/kg once every 3 weeks and PD-1 X LAG-3 BD at a flat dose of about 120 mg to about 800 mg once every 3 weeks. Another exemplary dosing regimen comprises administration of a B7-H3-ADC at a weight-based dose of about 3 mg/kg to about 5 mg/kg once every 3 weeks and PD-1 X LAG-3 BD at a flat dose of about 120 mg to about 800 mg once every 3 weeks. Another exemplary dosing regimen comprises administration of a B7-H3-ADC at a weight-based dose of about 3 mg/kg to about 4 mg/kg once every 3 weeks and PD-1 X LAG-3 BD at a flat dose of about 120 mg to about 800 mg once every 3 weeks. Another exemplary dosing regimen comprises administration of a B7-H3-ADC at a weight-based dose of about 4 mg/kg to about 5 mg/kg once every 3 weeks and PD-1 X LAG-3 BD at a flat dose of about 120 mg to about 800 mg once every 3 weeks. Another exemplary dosing regimen comprises administration of a B7-H3-ADC at a weight-based dose of about 2 mg/kg once every 3 weeks and PD-1 X LAG-3 BD at a flat dose of about 120 mg to about 800 mg once every 3 weeks. Another exemplary dosing regimen comprises administration of a B7-H3-ADC at a weight-based dose of about 3 mg/kg once every 3 weeks and PD-1 X LAG-3 BD at a flat dose of about 120 mg to about 800 mg once every 3 weeks. Another exemplary dosing regimen comprises administration of a B7-H3-ADC at a weight-based dose of about 3.25 mg/kg once every 3 weeks and PD-1 X LAG-3 BD at a flat dose of about 120 mg to about 800 mg once every 3 weeks. Another exemplary dosing regimen comprises administration of a B7-H3-ADC at a weight-based dose of about 3.5 mg/kg once every 3 weeks and PD-1 X LAG-3 BD at a flat dose of about 120 mg to about 800 mg once every 3 weeks. Another exemplary dosing regimen comprises administration of a B7-H3-ADC at a weight-based dose of about 3.75 mg/kg once every 3 weeks and PD-1 X LAG-3 BD at a flat dose of about 120 mg to about 800 mg once every 3 weeks. Another exemplary dosing regimen comprises administration of a B7-H3-ADC at a weight-based dose of about 4 mg/kg once every 3 weeks and PD-1 X LAG-3 BD at a flat dose of about 120 mg to about 800 mg once every 3 weeks. Another exemplary dosing regimen comprises administration of a B7-H3-ADC at a weight-based dose of about 4.25 mg/kg once every 3 weeks and PD-1 X LAG-3 BD at a flat dose of about 120 mg to about 800 mg once every 3 weeks. Another exemplary dosing regimen comprises administration of a B7-H3-ADC at a weight-based dose of about 4.5 mg/kg once every 3 weeks and PD-1 X LAG-3 BD at a flat dose of about 120 mg to about 800 mg once every 3 weeks. Another exemplary dosing regimen comprises administration of a B7-H3-ADC at a weight-based dose of about 4.75 mg/kg once every 3 weeks and PD-1 X LAG-3 BD at a flat dose of about 120 mg to about 800 mg once every 3 weeks. Another exemplary dosing regimen comprises administration of a B7-H3-ADC at a weight-based dose of about 5 mg/kg once every 3 weeks and PD-1 X LAG-3 BD at a flat dose of about 120 mg to about 800 mg once every 3 weeks.

Another exemplary dosing regimen comprises administration of a B7-H3-ADC at a weight-based dose of about 3 mg/kg to about 4 mg/kg once every 3 weeks and PD-1 X LAG-3 BD at a flat dose of about 300 mg once every 3 weeks. Another exemplary dosing regimen comprises administration of a B7-H3-ADC at a weight-based dose of about 4 mg/kg to about 5 mg/kg once every 3 weeks and PD-1 X LAG-3 BD at a flat dose of about 300 mg once every 3 weeks. Another exemplary dosing regimen comprises administration of a B7-H3-ADC at a weight-based dose of about 2 mg/kg once every 3 weeks and PD-1 X LAG-3 BD at a flat dose of about 300 mg once every 3 weeks. Another exemplary dosing regimen comprises administration of a B7-H3-ADC at a weight-based dose of about 3 mg/kg once every 3 weeks and PD-1 X LAG-3 BD at a flat dose of about 300 mg once every 3 weeks. Another exemplary dosing regimen comprises administration of a B7-H3-ADC at a weight-based dose of about 3.25 mg/kg once every 3 weeks and PD-1 X LAG-3 BD at a flat dose of about 300 mg once every 3 weeks. Another exemplary dosing regimen comprises administration of a B7-H3-ADC at a weight-based dose of about 3.5 mg/kg once every 3 weeks and PD-1 X LAG-3 BD at a flat dose of about 300 mg once every 3 weeks. Another exemplary dosing regimen comprises administration of a B7-H3-ADC at a weight-based dose of about 3.75 mg/kg once every 3 weeks and PD-1 X LAG-3 BD at a flat dose of about 300 mg once every 3 weeks. Another exemplary dosing regimen comprises administration of a B7-H3-ADC at a weight-based dose of about 4 mg/kg once every 3 weeks and PD-1 X LAG-3 BD at a flat dose of about 300 mg once every 3 weeks. Another exemplary dosing regimen comprises administration of a B7-H3-ADC at a weight-based dose of about 4.25 mg/kg once every 3 weeks and PD-1 X LAG-3 BD at a flat dose of about 300 mg once every 3 weeks. Another exemplary dosing regimen comprises administration of a B7-H3-ADC at a weight-based dose of about 4.5 mg/kg once every 3 weeks and PD-1 X LAG-3 BD at a flat dose of about 300 mg once every 3 weeks. Another exemplary dosing regimen comprises administration of a B7-H3-ADC at a weight-based dose of about 4.75 mg/kg once every 3 weeks and PD-1 X LAG-3 BD at a flat dose of about 300 mg once every 3 weeks. Another exemplary dosing regimen comprises administration of a B7-H3-ADC at a weight-based dose of about 5 mg/kg once every 3 weeks and PD-1 X LAG-3 BD at a flat dose of about 300 mg once every 3 weeks.

Another exemplary dosing regimen comprises administration of a B7-H3-ADC at a weight-based dose of about 3 mg/kg to about 4 mg/kg once every 3 weeks and PD-1 X LAG-3 BD at a flat dose of about 600 mg once every 3 weeks. Another exemplary dosing regimen comprises administration of a B7-H3-ADC at a weight-based dose of about 4 mg/kg to about 5 mg/kg once every 3 weeks and PD-1 X LAG-3 BD at a flat dose of about 600 mg once every 3 weeks. Another exemplary dosing regimen comprises administration of a B7-H3-ADC at a weight-based dose of about 2 mg/kg once every 3 weeks and PD-1 X LAG-3 BD at a flat dose of about 600 mg once every 3 weeks. Another exemplary dosing regimen comprises administration of a B7-H3-ADC at a weight-based dose of about 3 mg/kg once every 3 weeks and PD-1 X LAG-3 BD at a flat dose of about 600 mg once every 3 weeks. Another exemplary dosing regimen comprises administration of a B7-H3-ADC at a weight-based dose of about 3.25 mg/kg once every 3 weeks and PD-1 X LAG-3 BD at a flat dose of about 600 mg once every 3 weeks. Another exemplary dosing regimen comprises administration of a B7-H3-ADC at a weight-based dose of about 3.5 mg/kg once every 3 weeks and PD-1 X LAG-3 BD at a flat dose of about 600 mg once every 3 weeks. Another exemplary dosing regimen comprises administration of a B7-H3-ADC at a weight-based dose of about 3.75 mg/kg once every 3 weeks and PD-1 X LAG-3 BD at a flat dose of about 600 mg once every 3 weeks. Another exemplary dosing regimen comprises administration of a B7-H3-ADC at a weight-based dose of about 4 mg/kg once every 3 weeks and PD-1 X LAG-3 BD at a flat dose of about 600 mg once every 3 weeks. Another exemplary dosing regimen comprises administration of a B7-H3-ADC at a weight-based dose of about 4.25 mg/kg once every 3 weeks and PD-1 X LAG-3 BD at a flat dose of about 600 mg once every 3 weeks. Another exemplary dosing regimen comprises administration of a B7-H3-ADC at a weight-based dose of about 4.5 mg/kg once every 3 weeks and PD-1 X LAG-3 BD at a flat dose of about 600 mg once every 3 weeks. Another exemplary dosing regimen comprises administration of a B7-H3-ADC at a weight-based dose of about 4.75 mg/kg once every 3 weeks and PD-1 X LAG-3 BD at a flat dose of about 600 mg once every 3 weeks. Another exemplary dosing regimen comprises administration of a B7-H3-ADC at a weight-based dose of about 3.25 mg/kg once every 3 weeks and PD-1 X LAG-3 BD at a flat dose of about 600 mg once every 3 weeks.

It is particularly contemplated that in the above embodiments, administration occurs at the predetermined frequency or periodicity, or within 1-3 days of such scheduled dosing interval, such that administration occurs 1-3 day before, 1-3 days after, or on the day of a scheduled dose, e.g., once every 3 weeks (±3 days). It is specifically contemplated that in the above embodiments, a B7-H3-ADC and a PD-1 binding molecule are administered by IV infusion within a 24-hour period. In certain embodiments, a B7-H3-ADC and a PD-1 binding molecule are administered by IV infusion according to any of the above dosing regimens for a duration (i.e., course of treatment) of at least 1 month or more, at least 3 months or more, or at least 6 months or more, or at least 12 months or more. A treatment duration of at least 6 months or more, or for at least 12 months or more, or until remission of disease or unmanageable toxicity is observed, is particularly contemplated. In certain embodiments, treatment continues for a period of time after remission of disease.

In certain embodiments, a B7-H3-ADC and a PD-1 binding molecule are administered by IV infusion. The molecules are thus diluted (separately or together) into an infusion bag comprising a suitable diluent, e.g., 0.9% sodium chloride. Since infusion or allergic reactions may occur, premedication for the prevention of such infusion reactions is recommended and precautions for anaphylaxis should be observed during the antibody administration. In certain embodiments, the IV infusion to be administered to the subject over a period of between about 30 minutes and about 24 hours. In certain embodiments, the IV infusion is delivered over a period of about 30-240 minutes, about 30-180 minutes, about 30-120 minutes, or about 30-90 minutes, or over a period of about 60-90 minutes, or over a period of about 60-75 minutes, or over a lesser period, if the subject does not exhibit signs or symptoms of an adverse infusion reaction. In one embodiment, a B7-H3-ADC is administered by IV infusion over a period of about 60 minutes. In another embodiment, hPD-1 mAb-A is administered by IV infusion over a period of about 60 minutes. In a further embodiment, pembrolizumab is administered by IV infusion over a period of about 30 minutes. In a further embodiment, nivolumab is administered by IV infusion over a period of about 30 minutes. In a further embodiment PD-1 X LAG-3 BD is administered by IV infusion over a period of about 30-240 minutes or about 30-90 minutes.

Although, as discussed above, various dosing and administration routes may be employed in order to provide a B7-H3-ADC alone or a combination of a B7-H3-ADC and a PD-1 binding molecule to recipient subjects in need thereof in accordance with the present invention, certain combinations, dosing and administrative routes are particularly called out below for use in such treatment.

Accordingly, certain dosing regimens comprise administration of a B7-H3-ADC at a weight-based dose of from about 0.5 mg/kg to about 5 mg/kg either optionally in combination with a PD-1 binding molecule at a flat dose of from about 300-700 mg or at a weight-based dose of about 1 mg/kg to about 10 mg/kg, wherein such molecules are administered once every 3 weeks (±3 days). In certain embodiments, a B7-H3-ADC is administered at a weight-based dose of about 0.5 mg/kg, about 1 mg/kg, about 2 mg/kg, about 2.25 mg/kg, about 2.5 mg/kg, about 2.75 mg/kg, about 3 mg/kg, about 3.25 mg/kg about 3.5 mg/kg, about 3.75 mg/kg, about 4 mg/kg, about 4.25 mg/kg, about 4.5 mg/kg, about 4.75 mg/kg or about 5 mg/kg and the PD-1 binding molecule is administered at a flat dose of about 300 mg, about 375 mg, about 400 mg, about 500 mg, about 600 mg, or about 700 mg, or at a weight-based dose of about 1 mg/kg, about 3 mg/kg, about 5 mg/kg, or about 10 mg/kg.
(A) In certain embodiments, a B7-H3-ADC is administered at a weight-based dose of about 3 mg/kg. In such embodiments, if the PD-1 binding molecule that is to be administered is hPD-1 mAb-A, such hPD-1 mAb-A is administered at a flat dose of about 375 mg or about 500 mg. Alternatively, if in such embodiments, the PD-1 binding molecule that is administered is pembrolizumab, said pembrolizumab is administered at a flat dose of about 200 mg. Alternatively, if in such embodiments, the PD-1 binding molecule that is administered is nivolumab, said nivolumab is administered at a flat dose of 240 mg or 480 mg, or at weight-based dose of 3 mg/kg. Alternatively, if in such embodiments, the PD-1 binding molecule that is administered is PD-1 X LAG-3 BD, said PD-1 X LAG-3 BD is administered at a dose of 300 mg or about 600 mg.
(B) In certain embodiments, a B7-H3-ADC is administered at a weight-based dose of about 3.25 mg/kg. In such embodiments, if the PD-1 binding molecule that is to be administered is hPD-1 mAb-A, such hPD-1 mAb-A is administered at a flat dose of about 375 mg or about 500 mg. Alternatively, if in such embodiments, the PD-1 binding molecule that is administered is pembrolizumab, said pembrolizumab is administered at a flat dose of about 200 mg. Alternatively, if in such embodiments, the PD-1 binding molecule that is administered is nivolumab, said nivolumab is administered at a flat dose of 240 mg or 480 mg, or at weight-based dose of 3 mg/kg. Alternatively, if in such embodiments, the PD-1 binding molecule that is administered is PD-1 X LAG-3 BD, said PD-1 X LAG-3 BD is administered at a dose of 300 mg or about 600 mg.
(C) In certain embodiments, a B7-H3-ADC is administered at a weight-based dose of about 3.5 mg/kg. In such embodiments, if the PD-1 binding molecule that is to be administered is hPD-1 mAb-A, such hPD-1 mAb-A is administered at a flat dose of about 375 mg or about 500 mg. Alternatively, if in such embodiments, the PD-1 binding molecule that is administered is pembrolizumab, said pembrolizumab is administered at a flat dose of about 200 mg. Alternatively, if in such embodiments, the PD-1 binding molecule that is administered is nivolumab, said nivolumab is administered at a flat dose of 240 mg or 480 mg, or at weight-based dose of 3 mg/kg. Alternatively, if in such embodiments, the PD-1 binding molecule that is administered is PD-1 X LAG-3 BD, said PD-1 X LAG-3 BD is administered at a dose of 300 mg or about 600 mg.
(D) In certain embodiments, a B7-H3-ADC is administered at a weight-based dose of about 3.75 mg/kg. In such embodiments, if the PD-1 binding molecule that is to be administered is hPD-1 mAb-A, such hPD-1 mAb-A is administered at a flat dose of about 375 mg or about 500 mg. Alternatively, if in such embodiments, the PD-1 binding molecule that is administered is pembrolizumab, said pembrolizumab is administered at a flat dose of about 200 mg. Alternatively, if in such embodiments, the PD-1 binding molecule that is administered is nivolumab, said nivolumab is administered at a flat dose of 240 mg or 480 mg, or at weight-based dose of 3 mg/kg. Alternatively, if in such embodiments, the PD-1 binding molecule that is administered is PD-1 X LAG-3 BD, said PD-1 X LAG-3 BD is administered at a dose of 300 mg or about 600 mg.
(E) In certain embodiments, a B7-H3-ADC is administered at a weight-based dose of about 4 mg/kg. In such embodiments, if the PD-1 binding molecule that is to be administered is hPD-1 mAb-A, such hPD-1 mAb-A is administered at a flat dose of about 375 mg or about 500 mg. Alternatively, if in such embodiments, the PD-1 binding molecule that is administered is pembrolizumab, said pembrolizumab is administered at a flat dose of about 200 mg. Alternatively, if in such embodiments, the PD-1 binding molecule that is administered is nivolumab, said nivolumab is administered at a flat dose of 240 mg or 480 mg, or at weight-based dose of 3 mg/kg. Alternatively, if in such embodiments, the PD-1 binding molecule that is administered is PD-1 X LAG-3 BD, said PD-1 X LAG-3 BD is administered at a dose of 300 mg or about 600 mg.
(F) In certain embodiments, a B7-H3-ADC is administered at a weight-based dose of about 5 mg/kg. In such embodiments, if the PD-1 binding molecule that is to be administered is hPD-1 mAb-A, such hPD-1 mAb-A is administered at a flat dose of about 375 mg or about 500 mg. Alternatively, if in such embodiments, the PD-1 binding molecule that is administered is pembrolizumab, said pembrolizumab is administered at a flat dose of about 200 mg. Alternatively, if in such embodiments, the PD-1 binding molecule that is administered is nivolumab, said nivolumab is administered at a flat dose of 240 mg or 480 mg, or at weight-based dose of 3 mg/kg. Alternatively, if in such embodiments, the PD-1 binding molecule that is administered is PD-1 X LAG-3 BD, said PD-1 X LAG-3 BD is administered at a dose of 300 mg or about 600 mg.

In any of the above embodiments, a B7-H3-ADC and a PD-1 binding molecule are administered by IV infusion concurrently, sequentially, in an alternating manner, or at different times, within a 24-hour period. In any of the above embodiments, the PD-1 binding molecule is hPD-1 mAb-A or PD-1 X LAG-3 BD.

### XI. Embodiments of the Invention

The invention concerns in part the following non-limiting embodiments (E1-E119):
E1. A method of treating a cancer comprising administering a B7-H3-ADC to a subject in need thereof, wherein said method comprises administering said B7-H3-ADC to said subject at a dose of about 0.5 mg/kg to about 5 mg/kg once about every 3 weeks.
E2. The method of E1, wherein said method comprises administering said B7-H3-ADC to said subject at a dose of about 3 mg/kg to about 5 mg/kg once about every 3 weeks.
E3. The method of any one of E1-E2, wherein said method comprises administering said B7-H3-ADC to said subject at a dose of 3 mg/kg to about 4 mg/kg once about every 3 weeks.
E4. The method of any one of E1-E2, wherein said method comprises administering said B7-H3-ADC to said subject at a dose of about 4 mg/kg to about 5 mg/kg once about every 3 weeks.
E5. A method of treating a cancer comprising administering a B7-H3-ADC to a subject in need thereof, wherein said method comprises administering said B7-H3-ADC to said subject at a dose of about 0.5 mg/kg to about 5 mg/kg once about every 4 weeks.
E6. The method of E5, wherein said method comprises administering said B7-H3-ADC to said subject at a dose of about 3 mg/kg to about 5 mg/kg once about every 4 weeks.
E7. The method of any one of E5-E6, wherein said method comprises administering said B7-H3-ADC to said subject at a dose of 3 mg/kg to about 4 mg/kg once about every 4 weeks.
E8. The method of any one of E5-E6, wherein said method comprises administering said B7-H3-ADC to said subject at a dose of about 4 mg/kg to about 5 mg/kg once about every 4 weeks.
E9. The method of any one of E1-E8, wherein said method comprises administering said B7-H3-ADC to said subject at a dose of about 0.5 mg/kg, about 1 mg/kg, about 1.5 mg/kg, about 2 mg/kg, about 2.25 mg/kg, about 2.5 mg/kg, about 2.75 mg/kg, about 3 mg/kg, about 3.25 mg/kg, about 3.5 mg/kg, about 3.75 mg/kg, about 4 mg/kg, about 4.25 mg/kg, about 4.5 mg/kg, about 4.75 mg/kg or about 5 mg/kg.
E10. A method of treating a cancer comprising administering to a subject in need thereof:
   (A) a B7-H3-ADC; and
   (B) a PD-1 binding molecule,
   wherein said method comprises administering said B7-H3-ADC to the subject at a dose of about 0.5 mg/kg to about 5 mg/kg once every 3 weeks.
E11. A method of treating a cancer comprising administering to a subject in need thereof:
   (A) a B7-H3-ADC; and
   (B) a PD-1 binding molecule,
   wherein the method comprises administering said B7-H3-ADC to the subject at a dose of about 0.5 mg/kg to about 5 mg/kg once every 4 weeks.
E12. The method of any one of E1-E11, wherein said B7-H3-ADC is represented by the formula:

Ab-(LM)ₘ-(D)ₙ,
wherein:
   Ab is a humanized B7-H3 antibody or B7-H3 binding fragment thereof that binds to B7-H3 and comprises:
      (i) the CDRL1 sequence RASESIYSYLA (SEQ ID NO: 39), the CDRL2 sequence NTKTLPE (SEQ ID NO: 40) and the CDRL3 sequence QHHYGTPPWT (SEQ ID NO: 41) in its Variable Light Chain (VL) domain, and
      (ii) the CDRH1 sequence SYGMS (SEQ ID NO: 42), the CDRH2 sequence TINSGGSNTYY PDSLKG (SEQ ID NO: 43) and the CDRH3 sequence HDGGAMDY (SEQ ID NO: 44) in its Variable Heavy Chain (VH) domain;
   LM comprises at least one bond or a Linker Molecule that covalently links Ab and D;
   m is an integer between 0 and n and denotes the number of bonds or Linker Molecules of said B7-H3-ADC, except when LM is a bond, m is not 0;
   and
   n is an integer between 1 and 10 and denotes the number of cytotoxic duocarmycin moieties covalently linked to said B7-H3-ADC.
E13. The method of any one of E1-E12, wherein said B7-H3-ADC comprises:
   (I) the humanized VL Domain comprises the amino acid sequence of SEQ ID NO:17, and
   (II) the humanized VH Domain comprises the amino acid sequence of SEQ ID NO:18.
E14. The method of E12 or E13, wherein the Ab is an antibody.
E15. The method of any one of E12-E14, wherein the Ab further comprises an Fc Domain of a human IgG1.
E15.1. The method of any one of E12-E15, wherein the Ab comprises a Light Chain comprising the amino acid sequence of SEQ ID NO:19 and a Heavy Chain comprising the amino acid sequence of SEQ ID NO:20.
E16. The method of any one of E12-E15.1, wherein at least one of the LM is a Linker Molecule, and particularly wherein the LM Linker Molecule is a peptidic linker and/or a cleavable linker.
E17. The method of E16, wherein the peptidic linker is a valine-citrulline dipeptide linker.
E18. The method of any one of E12-E17, wherein the LM Linker Molecule further comprises a self-eliminating spacer between the cleavable linker and D.
E19. The method of any one of E12-E17, wherein the self-eliminating spacer comprises a para-aminobenzyloxycarbonyl moiety.
E20. The method of any one of E12-E19, wherein the LM further comprises a maleimide linker moiety between the cleavable linker and Ab.
E21. The method of any one of E12-E20, wherein LM is represented by the formula:

[V-(W)ₖ-(X)₁-A]
whereby the B7-H3-ADC is represented by the formula:

Ab-[V-(W)ₖ-(X)₁-A]-D
wherein:
   V is a cleavable linker,
   (W)ₖ-(X)₁ -A is an elongated, self-eliminating spacer system, that self-eliminates via a l,(4+2n)-elimination,
   W and X are each a l,(4+2n) electronic cascade spacer, being the same or different,
   A is either a spacer group of formula (Y)ₘ, wherein Y is a l,(4+2n) electronic cascade spacer, or a group of formula U, being a cyclisation elimination spacer,
   k, l and m are independently an integer of 0 (included) to 5 (included),
   n is an integer of 0 (included) to 10 (included),
   with the provisos that:
   when A is (Y)ₘ: then k+l+m≥1, and
   if k+l+m=l, then n>l;
   when A is U: then k+l≥1.
   W, X, and Y are independently selected from compounds having the formula:

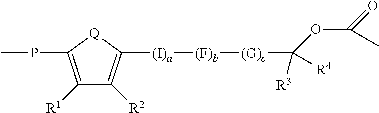

or the formula:

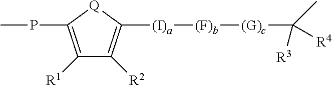

wherein: Q is —R⁵C═CR⁶—, S, O, NR⁵, —R⁵C═N—, or —N═CR⁵—
P is NR⁷, O or S
a, b, and c are independently an integer of 0 (included) to 5 (included);
I, F and G are independently selected from compounds having the formula:

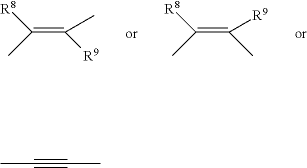

wherein R¹, R², R³, R⁴, R⁵, R⁶, R⁷, R⁸, and R⁹ independently represent H, C₁₋₆ alkyl, C₃₋₂₀ heterocyclyl, C₅₋₂₀ aryl, C₁₋₆ alkoxy, hydroxy (OH), amino (NH₂), mono-substituted amino (NRₓH), di-substituted amino (NRₓ¹Rₓ²), nitro (NO₂), halogen, CF₃, CN, CONH₂, SO₂Me, CONHMe, cyclic C₁₋₅ alkylamino, imidazolyl, C₁₋₆ alkylpiperazinyl, morpholino, thiol (SH), thioether (SRₓ), tetrazole, carboxy (COOH), carboxylate (COORₓ), sulphoxy (S(═O)₂OH), sulphonate (S(═O)₂ORₓ), sulphonyl (S(═O)₂Rₓ), sulphixy (S(═O)OH), sulphinate (S(═O)ORₓ), sulphinyl (S(═O)Rₓ), phosphonooxy (OP(═O)(OH)₂), and phosphate (OP(═O)(ORₓ)₂), where Rₓ, Rₓ¹ and Rₓ² are independently selected from a C₁₋₆ alkyl group, a C₃₋₂₀ heterocyclyl group or a C₅₋₂₀ aryl group, two or more of the substituents R¹, R², R³, R⁴, R⁵, R⁶, R⁷, R⁸, or R⁹ optionally being connected to one another to form one or more aliphatic or aromatic cyclic structures;
U is selected from compounds having the formula:

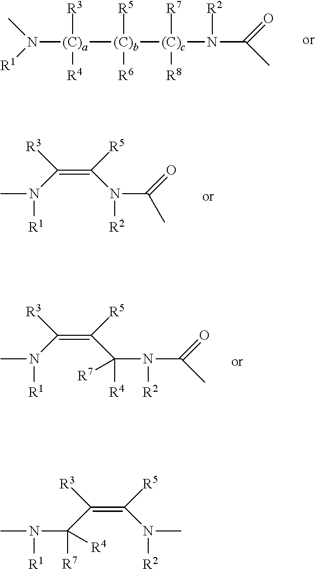

wherein:
a, b and c are independently selected to be an integer of 0 or 1;
provided that a+b+c=2 or 3;
R¹ and/or R² independently represent H, C1-6 alkyl, the alkyl being optionally substituted with one or more of the following groups: hydroxy (OH), ether (ORₓ), amino (NH₂), mono-substituted amino (NRₓH), disubstituted amino (NRₓ¹Rₓ²), nitro (NO₂), halogen, CF₃, CN, CONH₂, SO₂Me, CONHMe, cyclic C₁₋₅ alkylamino, imidazolyl C₁₋₆ alkylpiperazinyl, morpholino, thiol (SH), thioether (SR_{X}), tetrazole, carboxy (COOH), carboxylate (COORₓ), sulphoxy (S(═O)₂OH), sulphonate (S(═O)₂ORx), sulphonyl (S(═O)₂Rₓ), sulphixy (S(═O)OH), sulphinate (S(═O)ORx), sulphinyl (S(═O)Rx), phosphonooxy (OP(═O)(OH)₂), and phosphate (OP(═O)(ORₓ)₂), where Rₓ, Rₓ¹ and Rₓ² are selected from a C₁₋₆ alkyl group, a C₃₋₂₀ heterocyclyl group or a C₅₋₂₀ aryl group; and
R³, R⁴, R⁵, R⁶, R⁷ and R⁸ independently represent H, C₁₋₆ alkyl, C₃₋₂₀ heterocyclyl, C₅₋₂₀ aryl, C₁₋₆ alkoxy, hydroxy (OH), amino (NH₂), mono-substituted amino (NRₓH), disubstituted amino (NRₓ¹Rₓ²), nitro (NO₂), halogen, CF₃, CN, CONH₂, SO₂Me, CONHMe, cyclic C₁₋₅ alkylamino, imidazolyl, C₁₋₆ alkylpiperazinyl, morpholino, thiol (SH), thioether (SRₓ), tetrazole, carboxy (COOH), carboxylate (COORₓ), sulphoxy (S(═O)₂OH), sulphonate (S(═O)₂ORₓ), sulphonyl (S(═O)₂Rₓ), sulphixy (S(═O)OH), sulphinate (S(═O)ORₓ), sulphinyl (S(═O)Rₓ), phosphonooxy (OP(═O)(OH)₂), and phosphate (OP(═O)(ORₓ)₂), where Rₓ, Rₓ¹ and Rₓ² are selected from a C₁₋₆ alkyl group, a C₃₋₂₀ heterocyclyl group or a C₅₋₂₀ aryl group, and two or more of the substituents R¹, R², R³, R⁴, R⁵, R⁶, R⁷, or R⁸ are optionally connected to one another to form one or more aliphatic or aromatic cyclic structures.
E22. The method of any one of E12-E21, wherein the LM Linker Molecule comprises:
   (1) p-aminobenzyloxycarbonyl-p-aminobenzyloxycarbonyl;
   (2) p-aminobenzyloxycarbonyl-p-aminobenzyloxycarbonyl-p-aminobenzyloxycarbonyl;
   (3) p-ammocinnamyloxycarbonyl;
   (4) p-aminocinnamyloxycarbonyl-p-aminobenzyloxycarbonyl;
   (5) p-amino-benzyloxycarbonyl-p-aminocinnamyloxycarbonyl;
   (6) p-aminocinnamyloxycarbonyl-p-aminocinnamyloxycarbonyl;
   (7) p-aminophenylpentadienyloxycarbonyl;
   (8) p-aminophenylpentadienyloxycarbonyl-p-aminocinnamyloxycarbonyl;
   (9) p-aminophenylpentadienyloxycarbonyl-paminobenzyloxycarbonyl;
   (10) p-aminophenylpentadienyloxycarbonyl-p-aminophenylpentadienyloxycarbonyl;
   (11) p-aminobenzyloxycarbonyl(methylamino)ethyl(methylamino) carbonyl;
   (12) p-aminocinnamyloxycarbonyl(methylamino)ethyl(methylamino) carbonyl;
   (13) p-aminobenzyloxycarbonyl-p-aminobenzyloxycarbonyl(methylamino) ethyl(methylamino)carbonyl;
   (14) p-aminocinnamyloxycarbonyl-p-aminobenzyloxycarbonyl (methylamino)ethyl(methylamino)carbonyl;
   (15) p-aminobenzyloxycarbonyl-p-aminocinnamyloxycarbonyl (methylamino)ethyl(methylamino)-carbonyl;
   (16) p-aminocinnamyloxycarbonyl-p-aminocinnamyloxycarbonyl (methylamino)ethyl(methylamino)carbonyl;
   (17) p-aminobenzyloxycarbonyl-p-aminobenzyl;
   (18) p-aminobenzyloxycarbonyl-p-aminobenzyloxycarbonyl -p-aminobenzyl;
   (19) p-aminocinnamyl;
   (20) p-aminocinnamyloxycarbonyl-p-aminobenzyl;
   (21) p-aminobenzyloxycarbonyl-p-aminocinnamyl;
   (22) p-amino-cinnamyloxycarbonyl-p-aminocinnamyl;
   (23) p-aminophenylpentadienyl;
   (24) p-aminophenylpentadienyloxycarbonyl-p-aminocinnamyl;
   (25) p-aminophenylpentadienyloxycarbonyl-p-aminobenzyl; or
   (26) p-aminophenylpentadienyloxycarbonyl-p-aminophenylpentadienyl.
E23. The method of any one of E12-E22, wherein the LM Linker Molecule is conjugated to the side chain of an amino acid of a polypeptide chain of Ab and binds the Ab to a molecule of the cytotoxic duocarmycin moiety D, and in particular, wherein the cytotoxic duocarmycin moiety D.
E24. The method of any one of E12-E23, wherein the cytotoxic duocarmycin moiety D comprises a duocarmycin cytotoxin selected from the group consisting of duocarmycin A, duocarmycin B1, doucarmycin B2, duocarmycin C1, duocarmycin C2, duocarmycin D, duocarmycin SA, CC-1065, adozelesin, bizelesin, carzelesin (U-80244) and spiro-duocarmycin (DUBA)).
E25. The method of any one of E12-E24, wherein the cytotoxic duocarmycin moiety D comprises seco-duocarmycin.
E26. The method of any one of E12-E25, wherein the LM Linker Molecule is covalently linked to the Ab via reduced inter-chain disulfides.
E27. The method of any one of E1, E5, or E9-E26, wherein said B7-H3-ADC is administered at a dose of about 2 mg/kg.
E28. The method of any one of E1, E5, or E9-E26, wherein said B7-H3-ADC is administered at a dose of about 2.25 mg/kg.
E29. The method of any one of E1, E5, or E9-E26, wherein said B7-H3-ADC is administered at a dose of about 2.5 mg/kg.
E30. The method of any one of E1, E5, or E9-E26, wherein said B7-H3-ADC is administered at a dose of about 2.75 mg/kg.
E31. The method of any one of E1-E3, E5-E7, or E9-E26, wherein said B7-H3-ADC is administered at a dose of about 3 mg/kg.
E32. The method of any one of E1-E3, E5-E7, or E9-E26, wherein said B7-H3-ADC is administered at a dose of about 3.25 mg/kg.
E33. The method of any one of E1-E3, E5-E7, or E9-E26, wherein said B7-H3-ADC is administered at a dose of about 3.5 mg/kg.
E34. The method of any one of E1-E3, E5-E7, or E9-E26, wherein said B7-H3-ADC is administered at a dose of about 3.75 mg/kg.
E35. The method of any one of E1-E26, wherein said B7-H3-ADC is administered at a dose of about 4 mg/kg.
E36. The method of any one of E1-E26, wherein said B7-H3-ADC is administered at a dose of about 4.25 mg/kg.
E37. The method of any one of E1-E26, wherein said B7-H3-ADC is administered at a dose of about 4.5 mg/kg.
E38. The method of any one of E1-E26, wherein said B7-H3-ADC is administered at a dose of about 4.75 mg/kg.
E39. The method of any one of E1, E2, E4-E6, or E8-E26, wherein said B7-H3-ADC is administered at a dose of about 5 mg/kg.
E40. The method of any one of E1-E39, wherein said B7-H3-ADC is administered by intravenous (IV) infusion over a period of about 60 minutes.
E41. The method of any one of E1-E39, wherein said B7-H3-ADC is administered in combination with a therapeutically effective dose of a PD-1 binding molecule.
E42. The method of any one of E10, E11, or E41, wherein said PD-1 binding molecule is selected from the group consisting of an antibody, a single chain antibody, an Fab fragment, an F(ab′)2 fragment, an Fab′ fragment, an Fsc fragment, an Fv fragment, an scFv, an sc(Fv)2, and a diabody.
E43. The method of any one of E10, E11, E41, or E42, wherein said PD-1 binding molecule is selected from the group consisting of hPD-1 mAb-A, pembrolizumab, nivolumab and PD-1 X LAG-3 BD.
E44. The method of any one of E10, E11, or E41-E43, wherein said PD-1 binding molecule is hPD-1 mAb-A or PD-1 x LAG-3 BD.
E45. The method of any one of E10, E11, or E41-E44, wherein said PD-1 binding molecule comprises a variable heavy (VH) domain comprising VH complementarity determining region (CDR)1, VH CDR2 and VH CDR3, wherein
   the VH CDR1 comprises the amino acid sequence SYWMN (SEQ ID NO:23);
   the VH CDR2 comprises the amino acid sequence

(SEQ ID NO: 24)
VIHPSDSETWLDQKFKD;

the VH CDR3 comprises the amino acid sequence EHYGTSPFAY (SEQ ID NO:25); and
wherein the antibody comprises a variable light (VL) domain comprising VL CDR1, VL CDR2, and VL CDR3, wherein:
the VL CDR1 comprises the amino acid sequence

(SEQ ID NO: 26)
RASESVDNYGMSFMNW;

the VL CDR2 comprises the amino acid sequence AASNQGS (SEQ ID NO:27); and
the VL CDR3 comprises the amino acid sequence QQSKEVPYT (SEQ ID NO:28).
E46. The method of E45, wherein the VH domain of said PD-1 binding molecule comprises the amino acid sequence set forth in SEQ ID NO:32 and said VL domain comprises the amino acid sequence set forth in SEQ ID NO:31.
E47. The method of any one of E10, E11, or E41-E46, wherein said PD-1 binding molecule is hPD-1 mAb-A.
E48. The method of any one of E10, E11, or E41-E47, wherein the method comprises administering hPD-1 mAb-A once about every 3 weeks at a flat dose selected from the group consisting of about 375 mg, about 500 mg, and about 750 mg.
E49. The method of any one of E10, E11, or E41-E47, wherein the method comprises administering hPD-1 mAb-A once about every 4 weeks at a flat dose selected from the group consisting of about 375 mg, about 500 mg, and about 750 mg.
E50. The method of any one of E10, E11, or E41-E47, wherein said hPD-1 mAb-A is administered once about every 3 weeks at a flat dose of about 375 mg.
E51. The method of any one of E10, E11, or E41-E47, wherein said hPD-1 mAb-A is administered once about every 3 weeks at a flat dose of about 500 mg.
E52. The method of any one of E10, E11, E41, E47, E48 or E50, wherein said B7-H3-ADC is administered at a dose of about 3 mg/kg and said hPD-1 mAb-A is administered at a flat dose of about 375 mg once every 3 weeks.
E53. The method of any one of E10, E11, E41, E47, E48 or E50, wherein said B7-H3-ADC is administered at a dose of about 3.25 mg/kg and said hPD-1 mAb-A is administered at a flat dose of about 375 mg once every 3 weeks.
E54. The method of any one of E10, E11, E41, E47, E48 or E50, wherein said B7-H3-ADC is administered at a dose of about 3.5 mg/kg and said hPD-1 mAb A is administered at a flat dose of about 375 mg once every 3 weeks.
E55. The method of any one of E10, E11, E41, E47, E48 or E50, wherein said B7-H3-ADC is administered at a dose of about 3.75 mg/kg and said hPD-1 mAb A is administered at a flat dose of about 375 mg once every 3 weeks.
E56. The method of any one of E10, E11, E41, E47, E48 or E50, wherein said B7-H3-ADC is administered at a dose of about 4 mg/kg and said hPD-1 mAb-A is administered at a flat dose of about 375 mg once every 3 weeks.
E57. The method of any one of E10, E11, E41, E47, E48 or E50, wherein said B7-H3-ADC is administered at a dose of about 4.25 mg/kg and said hPD-1 mAb-A is administered at a flat dose of about 375 mg once every 3 weeks.
E58. The method of any one of E10, E11, E41, E47, E48 or E50, wherein said B7-H3-ADC is administered at a dose of about 4.5 mg/kg and said hPD-1 mAb-A is administered at a flat dose of about 375 mg once every 3 weeks.
E59. The method of any one of E10, E11, E41, E47, E48 or E50, wherein said B7-H3-ADC is administered at a dose of about 4.75 mg/kg and said hPD-1 mAb A is administered at a flat dose of about 375 mg once every 3 weeks.
E60. The method of any one of E10, E11, E41, E47, E48 or E50, wherein said B7-H3-ADC is administered at a dose of about 5 mg/kg and said hPD-1 mAb-A is administered at a flat dose of about 375 mg once every 3 weeks.
E61. The method of any one of E10, E11, E41, E47, or E49, wherein said B7-H3-ADC is administered at a dose of about 3 mg/kg and said hPD-1 mAb-A is administered at a flat dose of about 375 mg once every 4 weeks.
E62. The method of any one of E10, E11, E41, E47, or E49, wherein said B7-H3-ADC is administered at a dose of about 3.25 mg/kg and said hPD-1 mAb-A is administered at a flat dose of about 375 mg once every 4 weeks.
E63. The method of any one of E10, E11, E41, E47, or E49, wherein said B7-H3-ADC is administered at a dose of about 3.5 mg/kg and said hPD-1 mAb-A is administered at a flat dose of about 375 mg once every 4 weeks.
E64. The method of any one of E10, E11, E41, E47, or E49, wherein said B7-H3-ADC is administered at a dose of about 3.75 mg/kg and said hPD-1 mAb-A is administered at a flat dose of about 375 mg once every 4 weeks.
E65. The method of any one of E10, E11, E41, E47, or E49, wherein said B7-H3-ADC is administered at a dose of about 4 mg/kg and said hPD-1 mAb-A is administered at a flat dose of about 375 mg once every 4 weeks.
E66. The method of any one of E10, E11, E41, E47, or E49, wherein said B7-H3-ADC is administered at a dose of about 4.25 mg/kg and said hPD-1 mAb-A is administered at a flat dose of about 375 mg once every 4 weeks.
E67. The method of any one of E10, E11, E41, E47, or E49, wherein said B7-H3-ADC is administered at a dose of about 4.5 mg/kg and said hPD-1 mAb-A is administered at a flat dose of about 375 mg once every 4 weeks.
E68. The method of any one of E10, E11, E41, E47, or E49, wherein said B7-H3-ADC is administered at a dose of about 4.75 mg/kg and said hPD-1 mAb-A is administered at a flat dose of about 375 mg once every 4 weeks.
E69. The method of any one of E10, E11, E41, E47, or E49, wherein said B7-H3-ADC is administered at a dose of about 5 mg/kg and said hPD-1 mAb-A is administered at a flat dose of about 375 mg once every 4 weeks.
E70. The method of any one of E10, E11, E41-E69, wherein said hPD-1 mAb-A is administered by IV infusion over a period of about 60 minutes.
E71. The method of any one of E10, E11, or E41-E43, wherein said antibody that binds to human PD-1 is pembrolizumab.
E72. The method of any one of E41-E43 or E71, wherein said pembrolizumab is administered once about every 3 weeks at a flat dose of about 200 mg.
E73. The method of any one of E41-E43, E71, or E72, wherein said pembrolizumab is administered by IV infusion over a period of about 30 minutes.
E74. The method of any one of E10, E11, or E41-E43, wherein said PD-1 binding molecule is nivolumab.
E75. The method of any one of E41-E43 or E74, wherein said nivolumab is administered once about every 2 weeks at a flat dose of about 240 mg.
E76. The method of any one of E41-E43 or E74, wherein said nivolumab is administered once about every 4 weeks at a flat dose of about 480 mg.
E77. The method of any one of E41-E43 or E74-E76, wherein said nivolumab is administered by IV infusion over a period of about 30 minutes.
E78. The method of any one of E10, E11, E41-E44, wherein said PD-1 binding molecule is PD-1 X LAG-3 BD.
E78.1. The method of any one of E10, E11, E41-E44, wherein said PD-1 binding molecule is PD-1 X LAG-3 BD comprising a VH domain set forth in SEQ ID NO:32, a VL domain set forth in SEQ ID NO:31, a VH domain set forth in SEQ ID NO:46, and a VL domain set forth in SEQ ID NO:45.
E79. The method of any one of E43, E44, E78 or E78.1, wherein said PD-1 X LAG-3 BD comprises two polypeptide chains that comprise the amino acid sequence of SEQ ID NO:37 and two polypeptide chains that comprises the amino acid sequence of SEQ ID NO:38.
E80. The method of any one of E43, E44, E78, or E79, wherein said PD-1 X LAG-3 BD is administered at a flat dose of about 300 mg once every 2 weeks.
E81. The method of any one of E43, E44, E78, or E79, wherein said PD-1 X LAG-3 BD is administered at a flat dose of about 300 mg once every 3 weeks.
E82. The method of any one of E43, E44, E78, or E79, wherein said PD-1 X LAG-3 BD is administered at a flat dose of about 600 mg once every 2 weeks.
E83. The method of any one of E43, E44, E78, or E79, wherein said PD-1 X LAG-3 BD is administered at a flat dose of about 600 mg once every 3 weeks.
E84. The method of any one of E43, E44, or E78-E83, wherein said PD-1 X LAG-3 BD is administered by IV infusion over a period of 30-240 minutes.
E85. The method of any one of E43, E44, or E78-E83, wherein said PD-1 X LAG-3 BD is administered by IV infusion over a period of about 30-90 minutes.
E86. The method of any one of E43-E70, wherein said B7-H3-ADC and said hPD-1 mAb-A are administered sequentially to a subject in separate pharmaceutical compositions.
E87. The method of any one of E43-E71, wherein said pharmaceutical composition comprising said hPD-1 mAb-A is administered before the administration of the pharmaceutical composition comprising said B7-H3-ADC.
E88. The method of any one of E71-E73, wherein said B7-H3-ADC and said pembrolizumab are administered sequentially to a subject in separate pharmaceutical compositions.
E89. The method of any one of E74-E77, wherein said B7-H3-ADC and said nivolumab are administered sequentially to a subject in separate pharmaceutical compositions.
E90. The method of any one of E78-E85, wherein said B7-H3-ADC and said PD-1 X LAG-3 BD are administered sequentially to a subject in separate pharmaceutical compositions.
E91. The method of any one of E1-E90, wherein said B7-H3-ADC is provided in a pharmaceutical kit that comprises:
   (A) a pharmaceutical composition comprising from about 0.5 mg/ml to about 5 mg/ml of said B7-H3-ADC; and
   (B) an instructional material,
   wherein the instructional material instructs that the pharmaceutical composition comprising said B7-H3-ADC is to be administered optionally in combination with a pharmaceutical composition comprising a PD-1 binding molecule.
E92. The method of E91, wherein said PD-1 binding molecule is hPD-1 mAb-A, pembrolizumab, nivolumab or PD-1 X LAG-3 BD.
E93. The method of any one of E91 or E92, wherein in said pharmaceutical kit said B7-H3-ADC comprises:
   (I) the humanized VL Domain comprises the amino acid sequence of SEQ ID NO:17, and
   (II) the humanized VH Domain comprises the amino acid sequence of SEQ ID NO:18.
E94. The method of any one of E91-E93, wherein said instructional manual of said pharmaceutical kit instructs that said B7-H3-ADC is administered at a dose of about 0.5 mg/kg, about 1 mg/kg, about 1.5 mg/kg, about 2 mg/kg, about 2.25 mg/kg, about 2.5 mg/kg, about 2.75 mg/kg, about 3 mg/kg, about 3.25 mg/kg, about 3.5 mg/kg, about 3.75 mg/kg, about 4 mg/kg, about 4.25 mg/kg, about 4.5 mg/kg, about 4.75 mg/kg or about 5 mg/kg.
E95. The method of any one of E91-E94, wherein said instructional manual of said pharmaceutical kit instructs that said hPD-1 mAb-A is administered at a flat dose of about 375 mg or about 500 mg once every 3 weeks.
E96. The method of any one of E91-E94, wherein said instructional manual of said pharmaceutical kit instructs that said pembrolizumab is administered with a flat dose of about 200 mg of once every 3 weeks.
E97. The method of any one of E91-E94, wherein said instructional manual of said pharmaceutical kit instructs that said PD-1 X LAG-3 BD is administered at a flat dose of about 300 mg or about 600 mg once every 2 weeks or once every 3 weeks.
E98. The method of any one of E91-E95, wherein said instructional manual of said pharmaceutical kit instructs that said B7-H3-ADC and said hPD-1 mAb-A are administered by IV infusion over a period of about 60 minutes.
E99. The method of any one of E91-E94 or E97, wherein said instructional manual of said pharmaceutical kit instructs that said B7-H3-ADC is administered by IV infusion over a period of about 60 minutes and said PD-1 X LAG-3 BD is administered by IV infusion over a period of about 30-90 minutes.
E100. The method of any one of E91-E94 or E97, wherein said instructional manual of said pharmaceutical kit instructs that said B7-H3-ADC is administered by IV infusion over a period of about 60 minutes and said PD-1 X LAG-3 BD is administered by IV infusion over a period of about 30-240 minutes.
E101. The method of any one of E1-E100, wherein said B7-H3-ADC is to be administered optionally in combination with said PD-1 binding molecule for the treatment of a cancer in which B7-H3 is expressed.
E102. The method of any one of E1-E101, wherein said cancer is selected from the group consisting of: an adrenal gland cancer, an AIDS-associated cancer, an alveolar soft part sarcoma, an astrocytic tumor, an anal cancer ((e.g., squamous cell carcinoma of the anal canal (SCAC)), a bladder cancer, a bone cancer, a brain and spinal cord cancer, a metastatic brain tumor, a B-cell cancer, a breast cancer (e.g., a HER2+ breast cancer or triple negative breast cancer (TNBC)), a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, a gastric cancer, a gestational trophoblastic disease, a germ cell tumor, a head and neck cancer, a glioblastoma, a hematological malignancy, a hepatocellular carcinoma, an islet cell tumor, a Kaposi's Sarcoma, a kidney cancer, a leukemia (e.g., an acute myeloid leukemia), a liposarcoma/malignant lipomatous tumor, a liver cancer, a lymphoma, a lung cancer (e.g., a non-small-cell lung cancer (NSCLC)), a medulloblastoma, a melanoma, a meningioma, a mesothelioma pharyngeal cancer, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a pheochromocytoma, a pituitary tumor, a prostate cancer (e.g., a metastatic castration resistant prostate cancer (mCRPC)), a posterior uveal melanoma, a renal metastatic cancer, a rhabdoid tumor, a rhabdomyosarcoma, a sarcoma, a skin cancer, a small round blue cell tumor of childhood (e.g, neuroblastoma or rhabdomyosarcoma), a soft-tissue sarcoma, a squamous cell cancer (e.g., a squamous cell cancer of the head and neck (SCCHN), a stomach cancer, a synovial sarcoma, a testicular cancer, a thymic carcinoma, a thymoma, a thyroid cancer (e.g., a thyroid metastatic cancer), and a uterine cancer.
E103. The method of E102, wherein said cancer is a prostate cancer, an anal cancer, a squamous cell cancer, a breast cancer, a melanoma, or a lung cancer.
E104. The method of any one of E102 or E103, wherein said cancer is a prostate cancer.
E105. The method of any one of E102-E104, wherein said prostate cancer is mCRPC.
E106. The method of any one of E102 or E103, wherein said cancer is anal cancer.
E107. The method of E102 or E106, wherein said anal cancer is SCAC.
E108. The method of any one of E102 or E103, wherein said cancer is a squamous cell cancer.
E109. The method of any one of E102, E103, or E108, wherein said squamous cell cancer is SCCHN.
E110. The method of any one of E102 or E103, wherein said cancer is breast cancer.
E111. The method of any one of E102, E103, or E110, wherein said breast cancer is TNBC.
E112. The method of any one of E102 or E103, wherein said cancer is melanoma.
E113. The method of any one of E102, E103, or E112, wherein said melanoma is a uveal melanoma.
E114. The method of any one of E102 or E103, wherein said cancer is lung cancer.
E115. The method of any one of E102, E103, or E114, wherein said lung cancer is NSCLC.
E116. The method of any one of E1-E115, further comprising administering a therapeutically or prophylactically effective amount of one or more additional therapeutic agents or chemotherapeutic agents.
E117. The method of E116, wherein said chemotherapeutic agent is a platinum-based chemotherapeutic agent.
E118. The method of E116, wherein said chemotherapeutic agent is a taxane.
E119. The method of any one of E1-E119, wherein said subject in need thereof is a human.

### EXAMPLES

Having now generally described the invention, the same will be more readily understood through reference to the following Examples. The following examples illustrate various methods for compositions in the diagnostic or treatment methods of the invention. The examples are intended to illustrate, but in no way limit, the scope of the invention.

### Example 1

### B7-H3-ADC in Combination with a PD-1 Binding Molecule Exhibits Potent In Vivo Anti-Tumor Activity in C57BL/6 Mice

In order to further demonstrate the anti-tumor activity of a B7-H3-ADC of the present invention in combination with a PD-1 binding molecule, a B7-H3-ADC optionally in combination with an anti-PD-1 antibody (RMP1-14; BioXCell, Lebanon, NH, USA), were evaluated for in vivo toxicity in a C57BL/6 syngeneic mouse model using the murine MC38 colorectal tumor cell line transfected with human B7-H3 (“MC38/hB7-H3). In brief, ˜5×10⁵ tumor cells (suspended in 1:1 media and MATRIGEL®) were subcutaneously implanted into the flank of the C57BL/6 mice (Charles River Laboratories). When tumors had reached a volume of approximately 40-200 mm³ on day 15, the mice were randomized and B7-H3-ADC, anti-PD-1 antibody or control vehicle were administered intraperitoneally. In these studies, one dose of the B7-H3-ADC (5 mg/kg or 10 mg/kg) or control vehicle was administered on day 15. Anti-PD-1 antibody was administered at 20 mg/kg on days 15, 18, 21, 23, 25, 28, 30, 32, 35, and 37. Tumors were measured twice weekly by orthogonal measurements with electronic calipers, with tumor volumes calculated as: (length×width×height)/2. The tumor volume (relative to control) was determined (“T/C”). A finding that the tumor volume of treated animals had decreased to ≤5 mm³ during the study period was considered to denote a Complete Response (“CR”) and a Partial Response (“PR”) was defined when tumors were reduced by 50% or greater from day of dosing at any point during the study. Anti-tumor Activity was evaluated according to National Cancer Institute (NCI) standards; a T/C≤42% is the minimum level of anti-tumor activity, while a T/C value of >42% is inactive. A T/C<10% is considered highly active.

### In Vivo Activity Against MC38/hB7-113 Tumor Cells

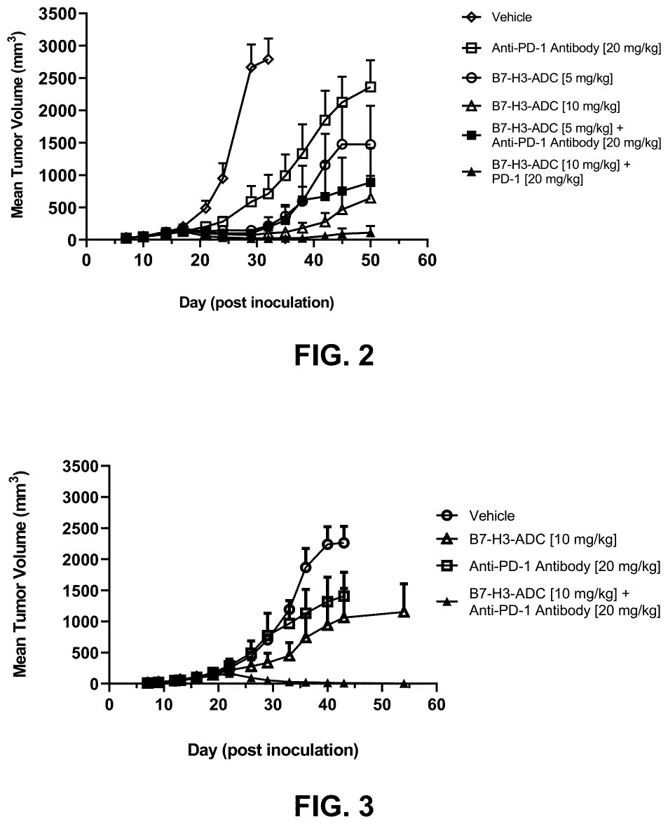

The results of this study with respect to subcutaneously implanted MC38/hB7-H3 colorectal cancer tumor cells are presented in Table 1 and in FIG. 2.

TABLE 1

| Treatment | Dose | % T/C at | | | Anti-Tumor |
|---|---|---|---|---|---|
| (Initial Dose on Day 15) | (mg/kg) | Day 29 | PR | CR | Activity |
| B7-H3-ADC | 5 | 5 | 1/6 | 0/6 | Highly Active |
| B7-H3-ADC | 10 | 4 | 4/6 | 2/6 | Highly Active |
| Anti-PD-1 Antibody | 20 | 22 | 0/6 | 0/6 | Active |
| B7-H3-ADC + | 5 | 6 | 4/6 | 3/6 | Highly Active |
| Anti-PD-1 Antibody | 20 | | | | |
| B7-H3-ADC + | 10 | 3 | 6/6 | 5/6 | Highly Active |
| Anti-PD-1 Antibody | 20 | | | | |

The results of this study demonstrate that the B7-H3-ADC exhibited dose-dependent in vivo anti-tumor activity toward B7-H3-positive tumors in a murine xenograft model of colorectal cancer. Complete responses were seen in 0/6 animals treated with 5 mg/kg B7-H3-ADC, whereas complete responses were seen in 2/6 animals treated with 10 mg/kg B7-H3-ADC. The combination of B7-H3-ADC with anti-PD-1 antibody enhanced the anti-tumor activity of B7-H3-ADC. Complete responses were seen in 3/6 animals treated with 5 mg/kg B7-H3-ADC+20 mg/kg anti-PD-1 antibody and in 5/6 animals treated with 10 mg/kg B7-H3-ADC+20 mg/kg anti-PD-1 antibody.

### Example 2

### B7-113-ADC in Combination with a PD-1 Binding Molecule Exhibits Potent In Vivo Anti-Tumor Activity in BALB/c Mice

In order to further demonstrate the anti-tumor activity of a B7-H3-ADC of the present invention in combination with a PD-1 binding molecule, a B7-H3-ADC optionally in combination with an anti-PD-1 antibody (RMP1-14; BioXCell, Lebanon, N.H., USA), were evaluated for in vivo toxicity in a BALB/c syngeneic mouse model using the murine CT26 colorectal tumor cell line transfected with human B7-H3 (“CT26/hB7-H3”). In brief, ˜5×10⁵ tumor cells (suspended in 1:1 media and MATRIGEL®) were subcutaneously implanted into the flank of the BALB/c mice (Charles River Laboratories). When tumors had reached a volume of approximately 40-100 mm³ on day 13, the mice were randomized and B7-H3-ADC, anti-PD-1 antibody or control vehicle were administered intraperitoneally. In these studies, one dose of the B7-H3-ADC (10 mg/kg) or control vehicle was administered on day 13. Anti-PD-1 antibody was administered at 20 mg/kg on days 13, 16, 19, 22, 26, 29, 33 and 36. Tumors were measured twice weekly by orthogonal measurements with electronic calipers, with tumor volumes calculated as: (length×width×height)/2. The tumor volume (relative to control) was determined (“T/C”). A finding that the tumor volume of treated animals had decreased to ≤5 mm³ during the study period was considered to denote a Complete Response (“CR”) and a Partial Response (“PR”) was defined when tumors were reduced by 50% or greater from day of dosing at any point during the study. Anti-tumor Activity was evaluated according to National Cancer Institute (NCI) standards; a T/C≤42% is the minimum level of anti-tumor activity, while a T/C value of >42% is inactive. A T/C<10% is considered highly active.

### In Vivo Activity Against CT26/hB7-113 Tumor Cells

The results of this study with respect to subcutaneously implanted CT26/hB7-H3 colorectal cancer tumor cells are presented in Table 2 and in FIG. 3.

TABLE 2

| Treatment | Dose | % T/C at | | | Anti-tumor |
|---|---|---|---|---|---|
| (Initial Dose on Day 13) | (mg/kg) | Day 36 | PR | CR | Activity |
| B7-H3-ADC | 10 | 40 | 4/7 | 2/7 | Active |
| B7-H3-ADC + | 10 | 1 | 7/7 | 7/7 | Highly Active |
| Anti-PD-1 Antibody | 20 | | | | |
| Anti-PD-1 Antibody | 20 | 61 | 1/7 | 1/7 | Inactive |

The results of this study demonstrate that the B7-H3-ADC exhibited in vivo anti-tumor activity toward B7-H3-positive tumors in a second murine xenograft model of colorectal cancer. Complete responses were seen in 2/7 animals treated with 10 mg/kg B7-H3-ADC. The combination of B7-H3-ADC with anti-PD-1 antibody enhanced the anti-tumor activity of B7-H3-ADC. Complete responses were seen in 7/7 animals treated with 10 mg/kg B7-H3-ADC+20 mg/kg anti-PD-1 antibody.

### Example 3

### Phase I Dose Studies

In order to determine the tolerability of patients to a B7-H3-ADC, a Phase I clinical study will be conducted. The study includes a Dose Escalation phase and a Cohort Expansion phase. The study is approved by the institutional review boards of each clinical site, and all patients sign a written-informed consent.

For the initial Dose Escalation and Dose Expansion cohorts, the B7-H3-ADC will be administered once every three weeks (Q3W). For purposes of the study, a six (6) week (42-day±3 days) cycle is used in which the B7-H3-ADC is administered Q3W starting on day 1 and day 22 of every 42-day cycle. Patients may receive multiple 6-week Q3W treatment cycles depending on tolerability and response to study treatments for a total of up to 18, 42-day cycles (i.e., approximately 2 years).

In additional Dose Escalation and Dose Expansion cohorts, the B7-H3-ADC and an anti-PD-1 antibody, hPD-1 mAb-A, are both administered once every three weeks (Q3W). For purposes of the study, three (3) week cycles (each being 21 days) are used in which B7-H3-ADC is administered on day 1 and day 22 of the first cycle and day 1 and day 22 of each subsequent cycle, and hPD-1 mAb-A is administered on day 22 of the first cycle and on day 1 and day 22 of every subsequent cycle. Patients may receive multiple 3-week Q3W treatment cycles depending on tolerability and response to study treatments for a total of up to 18, 42-day cycles (i.e., approximately 2 years).

In these studies, doses of the B7-H3-ADC are diluted in sterile 0.9% normal saline prior to administration over 60 minutes by intravenous (IV) infusion using a commercially available syringe or infusion pump. For syringe pump administration, the B7-H3-ADC is diluted to a concentration range of 0.1 mg/ml to 6.0 mg/ml. For infusion pump administration B7-H3-ADC is diluted to a concentration range of 0.5 mg/ml to 2.9 mg/ml.

In these studies, doses of hPD-1 mAb-A are diluted to a concentration range of 0.3 mg/ml to 12.0 mg/ml in sterile 0.9% normal saline prior to administration over 60 minutes by intravenous (IV) infusion using a commercially available syringe or infusion pump.

For both the Dose Escalation and Dose Expansion phases, tumor assessments will occur at day 42 (±3 days) of each cycle, for the first 4 cycles, and every other cycle thereafter. Antitumor activity is evaluated using: conventional Response Evaluation Criteria in Solid Tumors (RECIST), version 1.1 (Eisenhauer, E. A., et al. (2009) “New Response Evaluation Criteria In Solid Tumours: Revised RECIST Guideline (Version 1.1),” Eur. J. Cancer. 45(2):228-247); immune-related Response Evaluation Criteria in Solid Tumors (irRECIST) (Wolchok, J. D., et al. (2009) “Guidelines For The Evaluation Of Immune Therapy Activity In Solid Tumors: Immune-Related Response Criteria.” Clin. Cancer Res, 15:7412-7420); or the Revised International Working Group criteria (i.e., the Lugano Classification; Cheson, B. D. et al. (2014) “Recommendations For Initial Evaluation, Staging, And Response Assessment Of Hodgkin And Non-Hodgkin Lymphoma: The Lugano Classification.” J. Clin. Oncol 32:3059-3068) for response assessment, as applicable.

In the Dose Escalation phase, sequential escalating doses from 0.3 mg/kg up to 5 mg/kg are administered Q3W following a conventional 3+3+3 design: successive cohorts of 3 to 9 patients each are evaluated (Table 3). At various dose levels, patients assessed to not be evaluable for Dose Escalation purposes will be replaced. Additional patients are enrolled at multiple dose levels of interest to gain additional clinical experience. In the Dose Escalation phase, patients with unresectable, relapsed or refractory, locally advanced or metastatic solid tumors of any histology will be enrolled.

TABLE 3
B7-H3-ADC Dose Escalation Cohorts

| Cohort | B7-H3-ADC Dose (Q3W) | |
|---|---|---|
| Cohort -1 | 0.3 | mg/kg |
| Cohort 1 | 0.5 | mg/kg |
| Cohort 2 | 1 | mg/kg |
| Cohort 3 | 2 | mg/kg |
| Cohort 4 | 3 | mg/kg |
| Cohort 5 | 4 | mg/kg |
| Cohort 6 | 5 | mg/kg |

Based on the totality of the clinical data from the monotherapy (B7-H3-ADC alone) Dose Escalation phase, including but not limited to, observed clinical activity, peripheral receptor occupancy, and pharmacokinetics (PK), a maximum tolerated dose (MTD) administered Q3W, will be selected as the dosing regimen to be evaluated in a monotherapy Cohort Expansion phase and also in a combination Dose Escalation study of the B7-H3-ADC and hPD-1 mAb-A.

In the Dose Escalation phase of combination studies of the B7-H3-ADC and hPD-1 mAb-A, a flat dose of 375 mg of hPD-1 mAb-A will be administered Q3W following a conventional 3+3+3 design: successive cohorts of 3 to 9 patients with doses of the B7-H3-ADC as outlined in Table 4. At various dose levels, patients assessed to not be evaluable for Dose Escalation purposes will be replaced. Additional patients will be enrolled at multiple dose levels of interest to gain additional clinical experience. In the Dose Escalation phase, patients with unresectable, relapsed or refractory, locally advanced or metastatic solid tumors of any histology will be enrolled. Depending on the nature and timing of any observed toxicity, the dose of the B7-H3-ADC may be de-escalated to MTD -3 and hPD-1 mAb-A may be decreased to a flat dose of 250 mg, or both drugs may be adjusted to other levels at or below the Cohort 1 dose levels as deemed appropriate by study investigators.

TABLE 4
B7-H3-ADC and hPD-1 mAb-A Dose Escalation Cohorts

| Cohort | B7-H3-ADC (Q3W) | hPD-1 mAb-A (Q3W) |
|---|---|---|
| Cohort -1 | MTD-3 (TBD) | 250 mg (TBD) |
| Cohort 1 | MTD -2 | 375 mg |
| Cohort 2 | MTD -1 | 375 mg |
| Cohort 3 | MTD | 375 mg |

In the Cohort Expansion phase, patients with relapsed/refractory, unresectable locally advanced or metastatic SCCHN, mCRPC, TNBC, and uveal melanoma will receive the B7-H3-ADC alone at the MTD. Similarly, cohorts of patients with unresectable, locally advanced or metastatic SCCHN or mCRPC, based on the combination Dose Escalation studies, will be treated with the B7-H3-ADC in combination with hPD-1 mAb-A at the MTD dose based on the safety, PK and antitumor activity from the dose escalation phase of the study. Summary of Initial Findings

### Treatment-Related Adverse Events

The findings after treatment of 23 patients in Q3W dose escalation are provided. As shown in Table 6, treatment-related adverse events (TRAEs) occurred in 22/23 (91.7%) patients, most commonly neutropenia (n=6), lymphopenia (n=3), Palmar-plantar erythrodysaesthesia syndrome (n=5) and maculo-papular rash (n=3). The rate of Grade≥3 TRAEs was 58.3%. Three treatment-related serious adverse events occurred in 3 patients: 1) pneumonitis in a patient with concurrent bacterial pneumonia; 2) non-infectious gastroenteritis; and 3) stasis dermatitis in a patient with chronic venous insufficiency. One dose-limiting toxicity (“DLT”) of Grade 4 neutropenia that resolved to baseline was reported. No febrile neutropenia was observed.

TABLE 6
Summary of Adverse Events

| | Cohort N (% of N) | | | | | | | | | |
|---|---|---|---|---|---|---|---|---|---|---|
| | B7-H3-ADC Dose | | | | | | | | | |
| | 0.5 mg/kg | | 1 mg/kg | | 2 mg/kg | | 3 mg/kg | | All | |
| Patients Reporting at Least One: | (N = 3) | | (N = 6) | | (N = 7) | | (N = 7) | | (N = 23*) | |
| Adverse Event | 3 | (100) | 6 | (100) | 7 | (100) | 7 | (100) | 23 | (100) |
| Treatment-Related Adverse | 3 | (100) | 4 | (66.7) | 7 | (100) | 7 | (100) | 22 | (91.7) |
| Event ¹ | | | | | | | | | | |
| Adverse Event >= Grade 3 ² | 3 | (100) | 4 | (66.7) | 7 | (100) | 4 | (57.1) | 18 | (75.0) |
| Treatment-Related Adverse | 2 | (66.7) | 2 | (33.3) | 7 | (100) | 3 | (42.9) | 14 | (58.3) |
| Event >= Grade 3 ² | | | | | | | | | | |
| Serious Adverse Event | 1 | (33.3) | 1 | (16.7) | 3 | (42.9) | 0 | | 5 | (20.8) |
| Event that Resulted in Study | 1 | (33.3) | 1 | (16.7) | 3 | (42.9) | 0 | | 5 | (20.8) |
| Discontinuation | | | | | | | | | | |
| Event that Resulted in B7-H3- | 1 | (33.3) | 1 | (16.7) | 3 | (42.9) | 1 | (14.3) | 6 | (25.0) |
| ADC Withdrawal | | | | | | | | | | |
| Event that Resulted in B7-H3- | 0 | | 0 | | 1 | (14.3) | 2 | (28.6) | 3 | (12.5) |
| ADC Dose Reduction | | | | | | | | | | |
| Event that Resulted in B7-H3- | 1 | (33.3) | 0 | | 2 | (28.6) | 5 | (71.4) | 8 | (33.3) |
| ADC Interrupted | | | | | | | | | | |
| Fatal Adverse Event | 1 | (33.3) | 0 | | 0 | | 0 | | 1 | (4.2) |
| (pneumonitis) | | | | | | | | | | |
| Adverse Event of Special | 1 | (33.3) | 3 | (50.0) | 5 | (71.4) | 6 | (85.7) | 15 | (62.5) |
| Interest (AESI)- Infusion | | | | | | | | | | |
| Reaction³ | | | | | | | | | | |

¹ Includes events with causality assessments of ‘Possible’, ‘Probable’ or ‘Definite’.
² Based on CTCAE criteria version 4.0.3.

Based on the results from the dose escalation phase a dose of 3 mg/kg was selected as the treatment dose for the expansion cohorts.

Seventy-two patients have been treated with 3 mg/kg B7-H3-ADC (including dose escalation and dose expansion cohorts). Grade≥3 TRAEs occurred in 45.8% of the patients and included neutropenia (16.7%), lymphopenia (6.9%), anemia (4.2%), and palmer-planter erythrodysaesthesia syndrome (4.2%).

### Target Lesion Reduction

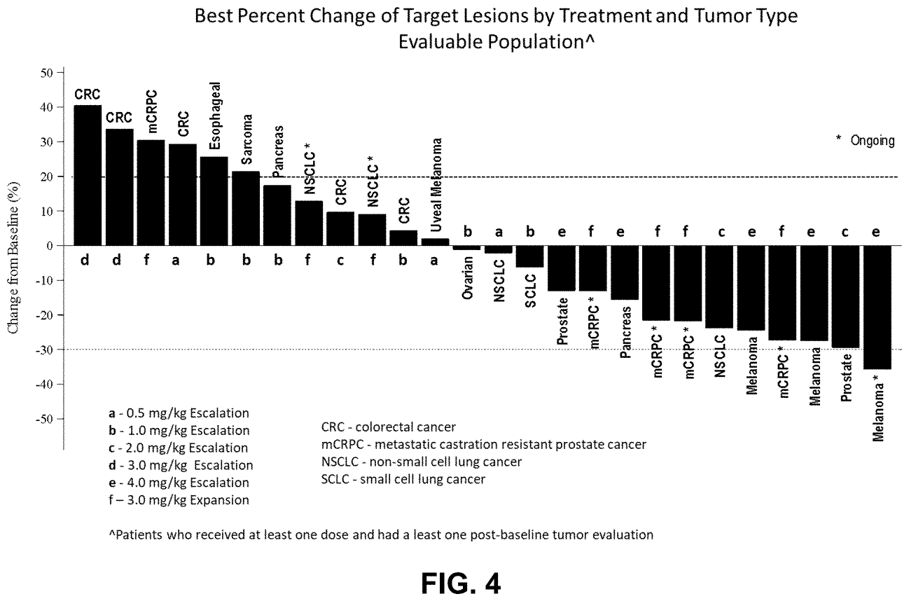

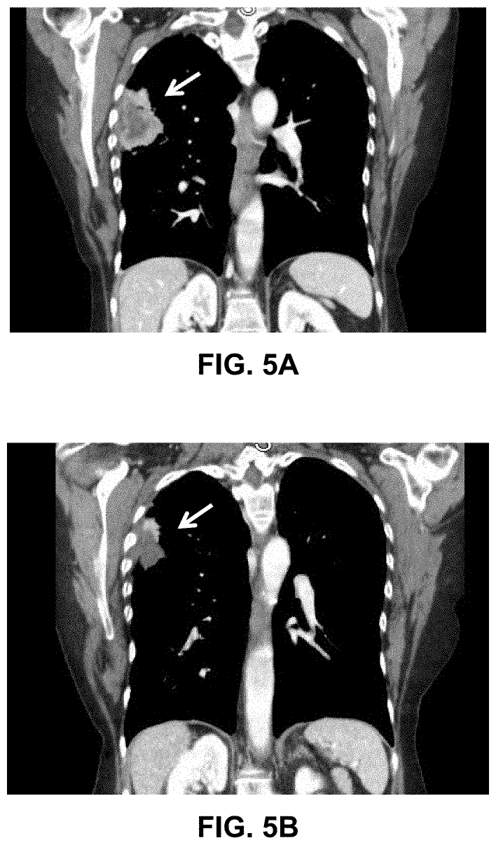

FIG. 4 presents a waterfall plot demonstrating the percent of reduction of target lesions among 26 response-evaluable dose escalation and cohort expansion patients receiving the B7-H3-ADC monotherapy at 0.5 mg/kg, 1 mg/kg, 2 mg/kg, 3 mg/kg, or 4 mg/kg Q3W. Patients were imaged every 6 weeks post-treatment in dose escalation and every 9 weeks post-treatment in cohort expansion. Data from patients that received at least one dose of B7-H3-ADC and had at least one post-baseline tumor evaluation are shown. Patients in the Dose Escalation cohorts included those with non-small cell lung cancer (NSCLC), uveal melanoma, melanoma, prostate cancer, metastatic castration-resistant prostate cancer (mCRPC), small cell lung cancer (SCLC), colorectal carcinoma (CRC), ovarian cancer, renal cell cancer (RCC), pancreatic cancer, sarcoma, and esophageal cancer. One metastatic castration-resistant prostate cancer (mCRPC) patient received 5 doses of the B7-H3-ADC; the first 4 doses were given at 2 mg/kg and the fifth dose at 1 mg/kg. This mCRPC patient had approximately 30% reduction in target lesions compared to baseline. One NSCLC patient received 6 doses of 2 mg/kg B7-H3-ADC and had a 24% reduction in target lesions compared to baseline. FIG. 5 shows computed tomography (CT) lung imaging scans of this patient following 2 doses of 2 mg/kg B7-H3-ADC Q3W. As noted by the clinical investigator, the lung lesion (noted by the arrow) in the anterior-posterior slice, evidenced a decrease of approximately 24%. FIG. 4 also shows the percent of reduction of target lesions among 7 response-evaluable cohort expansion patients receiving 3 mg/kg B7-H3-ADC. Two of the patients have NSCLC and 4 of the patients have mCRPC. Patients received at least one dose of B7-H3-ADC and had at least one post-baseline tumor evaluation. This study is ongoing and data is still maturing.

### Prostate Specific Antigen (PSA) Evaluation

Nine patients with mCRPC have been treated with 1 mg/kg, 2 mg/kg, 3 mg/kg, or 4mg/kg B7-H3-ADC Q3W in dose escalation. Blood samples were obtained from patients at baseline and at weeks 6, 12, and 19 to test levels of PSA. PSA levels were measured at clinical sites using routine testing. Five patients (71%) had a significant PSA level decline from baseline ranging from 50%-95%, including one patient with substantial regression of bone disease. A summary of the mCRPC patients, clinical responses, and PSA results are shown in Table 7.

TABLE 7
Summary of PSA Levels and Responses in Five mCRPC Patients

| | | | | | | Baseline | PSA Level | | % PSA | |
|---|---|---|---|---|---|---|---|---|---|---|
| | | | Dose B7- | | | PSA | (ng/ml) | | Decline | |
| | Type(s) of | Line of | H3-ADC | # of | # of | Level | (week | | from | Best |
| Patient | Lesion(s) | Therapy | Q3W | Doses | Cycles | (ng/ml) | measured) | | Baseline | Response |
| 1 | LN, AA, | 6 | 2 mg/kg | 3 | 4 | 114 | 46 | (16) | 60% | SD |
| | bone | | 1 mg/kg | 1 | | | | | | (~29%) |
| | lesions | | | | | | | | | |
| 2 | Bone only | 5 | 3 mg/kg | 2 | 2 | 60 | 0.5 | (24) | 98% | SD |
| | disease | | 2 mg/kg | 1 | | | | | | |
| | | | 1 mg/kg | 2 | | | | | | |
| 3 | Bone only | 5 | 3 mg/kg | 4 | 1 | 142 | 67 | (12) | 53% | SD |
| | disease | | | | | | | | | |
| 4 | Bone only | 3 | 3 mg/kg | 4 | 1 | 17 | 4.5 | (12) | 74% | SD |
| | disease | | | | | | | | | |
| 5 | Bone only | 6 | 3 mg/kg | 2 | 1 | 111 | 25 | (6) | 78% | SD |
| | disease | | | | | | | | | |

LN = lymph node;
AA = abdominal adenopathy

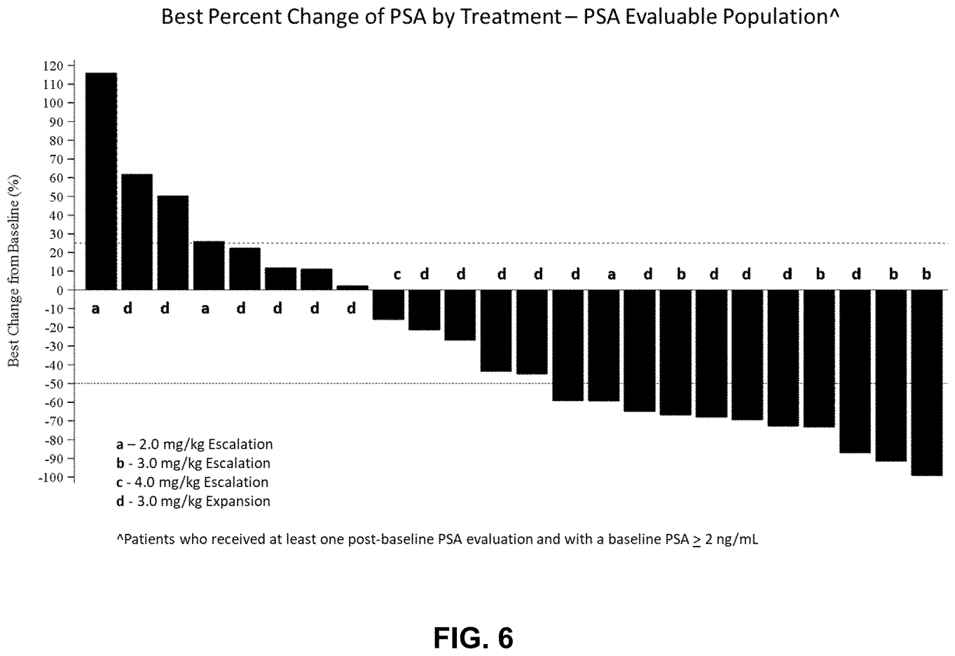

Nine patients with mCRPC have been treated with 2 mg/kg, 3.0 mg/kg, or 4 mg/kg B7-H3-ADC Q3W in dose escalation and 16 patients with mCRPC have been treated with 3.0 mg/kg B7-H3-ADC Q3W in cohort expansion. Eleven patients (46%) had a ≥50% PSA level decline from baseline ranging from 50%-95% as shown in FIG. 6. This study is ongoing and data is still maturing.

These data indicate that a B7-H3-ADC of the present invention demonstrated an acceptable safety profile and exhibited encouraging evidence of antitumor activity in multiple tumor types. These data also indicate a substantial reduction in PSA levels in mCRPC patients treated with B7-H3-ADC making mCRPC a viable cancer indication for further analysis in the cohort expansion phase of the study.

### Immunohistochemistry (IHC) Analysis of B7-H3 Expression

Expression of B7-H3 was analyzed in biopsies from prostate and other solid tumors in patients using immunohistochemistry (IHC). Eighteen patients had tissue samples which were evaluable for B7-H3 expression. The specific assay utilized was the B7-H3 (SP206) IHC assay from Ventana Medical Systems, Inc. (“Ventana”; Tuscon, Ariz.). Expression of B7-H3 in the cellular membrane of cells within the tumor and in the vasculature is represented by an H score of 0-3+ intensity. The percentage of cells at each staining intensity level was calculated, and finally, an H-score is assigned using the following formula:

H Score=[1×(% cells 1+)+2×(% cells 2+)+3×(% cells 3+)]

The range of H scores for B7-H3 expression was 82-279 with a median score of 200 in tumors. The range of B7-H3 expression in the vasculature was zero to 2+ and the median score was 2+.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method of treating a cancer comprising administering an anti-B7-H3 antibody-drug conjugate (B7-H3-ADC) to a subject in need thereof, wherein said method comprises administering said B7-H3-ADC to a subject at a dose of from about 0.5 mg/kg to about 5 mg/kg once every 3 weeks.

2. The method of claim 1, wherein said B7-H3-ADC is represented by the formula:
Ab-(LM)ₘ-(D)ₙ,
wherein:
Ab is a humanized B7-H3 antibody or B7-H3 binding fragment thereof that binds to B7-H3 and comprises:
(i) the CDRL1 sequence RASESIYSYLA (SEQ ID NO: 39), the CDRL2 sequence NTKTLPE (SEQ ID NO: 40) and the CDRL3 sequence QHHYGTPPWT (SEQ ID NO: 41) in its Variable Light Chain (VL) domain, and
(ii) the CDRH1 sequence SYGMS (SEQ ID NO: 42), the CDRH2 sequence TINSGGSNTYY PDSLKG (SEQ ID NO: 43) and the CDRH3 sequence HDGGAMDY (SEQ ID NO: 44) in its Variable Heavy Chain (VH) domain;
D is a cytotoxic duocarmycin moiety;
LM comprises at least one bond or a Linker Molecule that covalently links Ab and D;
m is an integer between 0 and n and denotes the number of bonds or Linker Molecules of said B7-H3-ADC, except when LM is a bond, m is not 0;
and
n is an integer between 1 and 10 and denotes the number of cytotoxic duocarmycin moieties covalently linked to said B7-H3-ADC molecule.

3. The method of claim 2, wherein said Ab comprises:
(i) a humanized Variable Light Chain (VL) domain comprising the amino acid sequence of SEQ ID NO:17; and
(ii) a humanized Variable Heavy Chain (VH) domain comprising the amino acid sequence of SEQ ID NO:18.

4. The method of any one of claim 2 or 3, wherein said Ab further comprises an Fc Domain of a human IgG.

5. The method of claim 4, wherein said human IgG is a human IgG1, IgG2, IgG3, or IgG4.

6. The method of any one of claim 4 or 5, wherein said Fc Domain is a variant Fc Domain that comprises:
(a) one or more amino acid modifications that reduces the affinity of the variant Fc Domain for an FcγR; and/or
(b) one or more amino acid modifications that enhances the serum half-life of the variant Fc Domain.

7. The method of claim 6, wherein said modifications that reduces the affinity of the variant Fc Domain for an FcγR comprise the substitution of L234A; L235A; or L234A and L235A, wherein said numbering is that of the EU index as in Kabat.

8. The method of any one of claim 5 or 6, wherein said modifications that that enhances the serum half-life of the variant Fc Domain comprise the substitution of M252Y; M252Y and S254T; M252Y and T256E; M252Y, S254T and T256E; or K288D and H435K, wherein said numbering is that of the EU index as in Kabat.

9. The method of any one of claims 2-8, wherein at least one of said LM is a Linker Molecule.

10. The method of any one of claims 2-9, wherein said LM Linker Molecule is a peptidic linker.

11. The method of claim 10, wherein said peptidic linker is a valine-citrulline dipeptide linker.

12. The method of any one of claims 2-11, wherein said LM Linker Molecule further comprises a self-eliminating spacer between the cleavable linker and D.

13. The method of claim 12, wherein said self-eliminating spacer comprises a para-aminobenzyloxycarbonyl moiety.

14. The method of any one of claims 2-13, wherein said Linker Molecule further comprises a maleimide linker moiety between the cleavable linker and Ab.

15. The method of any one of claims 1-14, wherein LM is represented by the formula:
[V-(W)ₖ-(X)₁-A]
whereby said B7-H3-ADC is represented by the formula:
Ab-[V-(W)ₖ-(X)₁-A]-D
wherein:
V is a cleavable linker,
(W)ₖ-(X)₁-A is an elongated, self-eliminating spacer system, that self-eliminates via a l,(4+2n)-elimination,
W and X are each a l,(4+2n) electronic cascade spacer, being the same or different,
A is either a spacer group of formula (Y)ₘ, wherein Y is a l,(4+2n) electronic cascade spacer, or a group of formula U, being a cyclisation elimination spacer,
k, l and m are independently an integer of 0 (included) to 5 (included),
n is an integer of 0 (included) to 10 (included),
with the provisos that:
when A is (Y)ₘ: then k+l+m≥1, and
if k+l+m=1, then n>l;
when A is U: then k+l≥1.
W, X, and Y are independently selected from compounds having the formula: 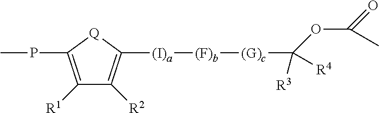
or the formula: 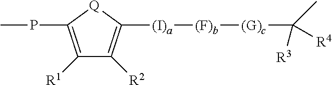
wherein: Q is —R⁵C═CR⁶—, S, O, NR⁵, —R⁵C═N—, or —N═CR⁵—
P is NR⁷, O or S
a, b, and c are independently an integer of 0 (included) to 5 (included);
I, F and G are independently selected from compounds having the formula: 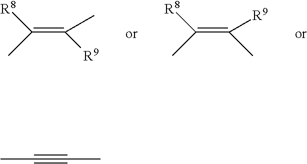
wherein R¹, R², R³, R⁴, R⁵, R⁶, R⁷, R⁸, and R⁹ independently represent H, C₁₋₆ alkyl, C₃₋₂₀ heterocyclyl, C₅₋₂₀ aryl, C₁₋₆ alkoxy, hydroxy (OH), amino (NH2), mono-substituted amino (NRₓH), di-substituted amino (NRₓ¹Rₓ²), nitro (NO₂), halogen, CF₃, CN, CONH₂, SO₂Me, CONHMe, cyclic C₁₋₅ alkylamino, imidazolyl, C₁₋₆ alkylpiperazinyl, morpholino, thiol (SH), thioether (SRₓ), tetrazole, carboxy (COOH), carboxylate (COORₓ), sulphoxy (S(═O)₂OH), sulphonate (S(═O)₂ORₓ), sulphonyl (S(═O)₂Rₓ), sulphixy (S(═O)OH), sulphinate (S(═O)ORₓ), sulphinyl (S(═O)Rₓ), phosphonooxy (OP(═O)(OH)₂), and phosphate (OP(═O)(ORₓ)₂), where Rₓ, Rₓ¹ and Rₓ² are independently selected from a C₁₋₆ alkyl group, a C₃₋₂₀ heterocyclyl group or a C₅₋₂₀ aryl group, two or more of the substituents R¹, R², R³, R⁴, R⁵, R⁶, R⁷, R⁸, or R⁹ optionally being connected to one another to form one or more aliphatic or aromatic cyclic structures;
U is selected from compounds having the formula: 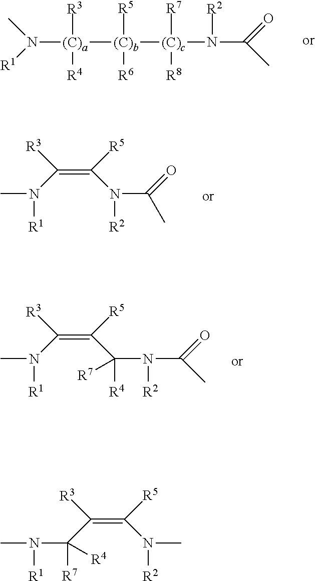
wherein:
a, b and c are independently selected to be an integer of 0 or 1;
provided that a+b+c=2 or 3;
R¹ and/or R² independently represent H, C1-6 alkyl, the alkyl being optionally substituted with one or more of the following groups: hydroxy (OH), ether (ORₓ), amino (NH₂), mono-substituted amino (NRₓH), disubstituted amino (NRₓ¹Rₓ²), nitro (NO₂), halogen, CF₃, CN, CONH₂, SO₂Me, CONHMe, cyclic C₁₋₅ alkylamino, imidazolyl, C₁₋₆ alkylpiperazinyl, morpholino, thiol (SH), thioether (SR_{X}), tetrazole, carboxy (COOH), carboxylate (COOR_{X}), sulphoxy (S(═O)₂OH), sulphonate (S(═O)₂ORx), sulphonyl (S(═O)₂Rx), sulphixy (S(═O)OH), sulphinate (S(═O)ORx), sulphinyl (S(═O)Rx), phosphonooxy (OP(═O)(OH)₂), and phosphate (OP(═O)(ORₓ)₂), where Rₓ, Rₓ¹ and Rₓ² are selected from a C₁₋₆ alkyl group, a C₃₋₂₀ heterocyclyl group or a C₅₋₂₀ aryl group; and
R³, R⁴, R⁵, R⁶, R⁷ and R⁸ independently represent H, C₁₋₆ alkyl, C₃₋₂₀ heterocyclyl, C₅₋₂₀ aryl, C₁₋₆ alkoxy, hydroxy (OH), amino (NH₂), mono-substituted amino (NRₓH), disubstituted amino (NRₓ¹Rₓ²), nitro (NO₂), halogen, CF₃, CN, CONH₂, SO₂Me, CONHMe, cyclic C₁₋₅ alkylamino, imidazolyl, C₁₋₆ alkylpiperazinyl, morpholino, thiol (SH), thioether (SRₓ), tetrazole, carboxy (COOH), carboxylate (COORₓ), sulphoxy (S(═O)₂OH), sulphonate (S(═O)₂ORₓ), sulphonyl (S(═O)₂Rₓ), sulphixy (S(═O)OH), sulphinate (S(═O)ORₓ), sulphinyl (S(═O)Rₓ), phosphonooxy (OP(═O)(OH)₂), and phosphate (OP(═O)(ORₓ)₂), where Rₓ, Rₓ¹ and Rₓ² are selected from a C₁₋₆ alkyl group, a C₃₋₂₀ heterocyclyl group or a C₅₋₂₀ aryl group, and two or more of the substituents R¹, R², R³, R⁴, R⁵, R⁶, R⁷, or R⁸ are optionally connected to one another to form one or more aliphatic or aromatic cyclic structures.

16. The method of claim 15, wherein said LM linker molecule comprises:
(1) p-aminobenzyloxycarbonyl-p-aminobenzyloxycarbonyl;
(2) p-aminobenzyloxycarbonyl-p-aminobenzyloxycarbonyl-p-aminobenzyloxycarbonyl;
(3) p-ammocinnamyloxycarbonyl;
(4) p-aminocinnamyloxycarbonyl-p-aminobenzyloxycarbonyl;
(5) p-amino-benzyloxycarbonyl-p-aminocinnamyloxycarbonyl;
(6) p-aminocinnamyloxycarbonyl-p-aminocinnamyloxycarbonyl;
(7) p-aminophenylpentadienyloxycarbonyl;
(8) p-aminophenylpentadienyloxycarbonyl-p-aminocinnamyloxycarbonyl;
(9) p-aminophenylpentadienyloxycarbonyl-paminobenzyloxycarbonyl;
(10) p-aminophenylpentadienyloxycarbonyl-p-aminophenylpentadienyloxycarbonyl;
(11) p-aminobenzyloxycarbonyl(methylamino)ethyl(methylamino) carbonyl;
(12) p-aminocinnamyloxycarbonyl(methylamino)ethyl(methylamino) carbonyl;
(13) p-aminobenzyloxycarbonyl-p-aminobenzyloxycarbonyl(methylamino) ethyl(methylamino)carbonyl;
(14) p-aminocinnamyloxycarbonyl-p-aminobenzyloxycarbonyl (methylamino)ethyl(methylamino)carbonyl;
(15) p-aminobenzyloxycarbonyl-p-aminocinnamyloxycarbonyl (methylamino)ethyl(methylamino)-carbonyl;
(16) p-aminocinnamyloxycarbonyl-p-aminocinnamyloxycarbonyl (methylamino)ethyl(methylamino)carbonyl;
(17) p-aminobenzyloxycarbonyl-p-aminobenzyl;
(18) p-aminobenzyloxycarbonyl-p-aminobenzyloxycarbonyl-p-aminobenzyl;
(19) p-aminocinnamyl;
(20) p-aminocinnamyloxycarbonyl-p-aminobenzyl;
(21) p-aminobenzyloxycarbonyl-p-aminocinnamyl;
(22) p-amino-cinnamyloxycarbonyl-p-aminocinnamyl;
(23) p-aminophenylpentadienyl;
(24) p-aminophenylpentadienyloxycarbonyl-p-aminocinnamyl;
(25) p-aminophenylpentadienyloxycarbonyl-p-aminobenzyl; or
(26) p-aminophenylpentadienyloxycarbonyl-p-aminophenylpentadienyl.

17. The method of any one of claims 2-16, wherein said LM Linker Molecule is conjugated to the side chain of an amino acid of a polypeptide chain of Ab and binds the Ab to a molecule of the cytotoxic duocarmycin moiety D.

18. The method of any one of claims 2-17, wherein said cytotoxic duocarmycin moiety D comprises a duocarmycin cytotoxin selected from the group consisting of: duocarmycin A, duocarmycin B1, duocarmycin B2, duocarmycin C1, duocarmycin C2, duocarmycin D, duocarmycin SA, CC-1065, adozelesin, bizelesin, carzelesin (U-80244), seco-duocarmycin and spiro-duocarmycin (DUBA).

19. The method of claim 18, wherein the cytotoxic duocarmycin moiety D comprises seco-duocarmycin.

20. The method of any of claims 2-19, wherein said LM Linker Molecule is covalently linked to the Ab via reduced inter-chain disulfides.

21. The method of any one of claims 1-20, wherein said B7-H3-ADC is administered at a dose of about 3 mg/kg.

22. The method of any one of claims 1-20, wherein said B7-H3-ADC is administered at a dose of about 3.5 mg/kg.

23. The method of any one of claims 1-20, wherein said B7-H3-ADC is administered at a dose of about 4 mg/kg.

24. The method of any one of claims 1-23, wherein said B7-H3-ADC is administered by intravenous (IV) infusion.

25. The method of claim 24, wherein said IV infusion is over a period of about 60 minutes.

26. The method of any one of claims 1-25, wherein said B7-H3-ADC is administered in combination with a therapeutically effective dose of a PD-1 binding molecule.

27. The method of any one of claims 1-26, wherein said cancer is selected from the group consisting of: an adrenal gland cancer, an AIDS-associated cancer, an alveolar soft part sarcoma, an astrocytic tumor, an anal cancer, squamous cell carcinoma of the anal canal (SCAC), a bladder cancer, a bone cancer, a brain and spinal cord cancer, a metastatic brain tumor, a B-cell cancer, a breast cancer, a HER2+ breast cancer, triple negative breast cancer (TNBC), a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, a gastric cancer, a gestational trophoblastic disease, a germ cell tumor, a head and neck cancer, a glioblastoma, a hematological malignancy, a hepatocellular carcinoma, an islet cell tumor, a Kaposi's Sarcoma, a kidney cancer, a leukemia, an acute myeloid leukemia, a liposarcoma/malignant lipomatous tumor, a liver cancer, a lymphoma, a lung cancer, a non-small-cell lung cancer (NSCLC), a medulloblastoma, a melanoma, a meningioma, a mesothelioma pharyngeal cancer, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a metastatic castration resistant prostate cancer (mCRPC), a posterior uveal melanoma, a renal metastatic cancer, a rhabdoid tumor, a rhabdomyosarcoma, a sarcoma, a skin cancer, a small round blue cell tumor of childhood, a neuroblastoma, a soft-tissue sarcoma, a squamous cell cancer, a squamous cell cancer of the head and neck (SCCHN), a stomach cancer, a synovial sarcoma, a testicular cancer, a thymic carcinoma, a thymoma, a thyroid cancer, a thyroid metastatic cancer, and a uterine cancer.

28. The method of claim 27, wherein said cancer is selected from the group consisting: of adrenal cancer, anal cancer, SCAC, bladder cancer, breast cancer, colorectal cancer, gastric cancer, glioblastoma, kidney cancer, lung cancer, NSCLC, acute lymphocytic leukemia, acute myeloid leukemia, chronic lymphocytic leukemia, chronic myeloid leukemia, hairy cell leukemia, Burkett's lymphoma, diffuse large B cell lymphoma, follicular lymphoma, mantle cell lymphoma, marginal zone lymphoma, mesothelioma pharyngeal cancer, non-Hodgkin's lymphoma, small lymphocytic lymphoma, multiple myeloma, melanoma, ovarian cancer, pancreatic cancer, a posterior uveal melanoma, prostate cancer, mCRPC, skin cancer, renal cell carcinoma, small round blue cell tumors of childhood, neuroblastoma, rhabdomyosarcoma, squamous cell cancer, SCCHN, testicular cancer, thyroid cancer, thyroid metastatic cancer, and uterine cancer.

29. The method of any one of claim 27 or 28, wherein said cancer is prostate cancer.

30. The method of any one of claims 27-29, wherein said prostate cancer is mCRPC.

31. The method of any one of claim 27 or 28, wherein said cancer is anal cancer.

32. The method of any one of claim 27, 28, or 31, wherein said anal cancer is SCAC.

33. The method of any one of claim 28 or 28, wherein said cancer is a squamous cell cancer.

34. The method of any one of claim 27, 28, or 33, wherein said squamous cell cancer is SCCHN.

35. The method of any one of claim 27 or 28, wherein said cancer is breast cancer.

36. The method of any one of claim 27, 28, or 35, wherein said breast cancer is TNBC.

37. The method of any one of claim 27 or 28, wherein said cancer is melanoma.

38. The method of any one of claim 27, 28, or 37, wherein said melanoma is a uveal melanoma.

39. The method of any one of claim 27 or 28, wherein said cancer is lung cancer.

40. The method of any one of claim 27, 28, or 39, wherein said lung cancer is NSCLC.

41. The method of any one of claims 1-40, further comprising administering a therapeutically or prophylactically effective amount of one or more additional therapeutic agents or chemotherapeutic agents.

42. The method of claim 41, wherein said chemotherapeutic agent is a platinum-based chemotherapeutic agent.

43. The method of claim 41, wherein said chemotherapeutic agent is a taxane.

44. The method of any one of claims 1-43, wherein said subject in need thereof is a human.


SEQUENCE LISTING (46 sequences)

SEQ ID NO: 1 - protein, 107 aa, Homo sapiens [sequence omitted]

SEQ ID NO: 2 - protein, 104 aa, Homo sapiens
QPKAAPSVTLFPPSSEELQANKATLVCLISDFYPGAVTVAWKADSSPVKAGVETTPSKQSNNKYAASSYLSLTPEQWKSHRSYSCQVTHEGSTVEKTVAPTECS

SEQ ID NO: 3 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 4 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 5 - protein, 15 aa, Homo sapiens
EPKSCDKTHTCPPCP

SEQ ID NO: 6 - protein, 12 aa, Homo sapiens
ESKYGPPCPSCP

SEQ ID NO: 7 - protein, 12 aa, Artificial Sequence
  Description of Artificial Sequence Synthetic peptide
ESKYGPPCPPCP

SEQ ID NO: 8 - protein, 217 aa, Homo sapiens
  MOD_RES (217)..(217): K or absent
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 9 - protein, 217 aa, Homo sapiens
  MOD_RES (217)..(217): K or absent
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 10 - protein, 8 aa, Artificial Sequence
  Description of Artificial Sequence Synthetic peptide
GGGSGGGG

SEQ ID NO: 11 - protein, 6 aa, Artificial Sequence
  Description of Artificial Sequence Synthetic peptide
GGCGGG

SEQ ID NO: 12 - protein, 28 aa, Artificial Sequence
  Description of Artificial Sequence Synthetic peptide
EVAACEKEVAALEKEVAALEKEVAALEK

SEQ ID NO: 13 - protein, 28 aa, Artificial Sequence
  Description of Artificial Sequence Synthetic peptide
KVAACKEKVAALKEKVAALKEKVAALKE

SEQ ID NO: 14 - protein, 217 aa, Artificial Sequence
  Description of Artificial Sequence Synthetic polypeptide
  MOD_RES (217)..(217): K or absent
APEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 15 - protein, 108 aa, Artificial Sequence
  Description of Artificial Sequence Synthetic polypeptide
DIQMTQSPASLSVSVGETVTITCRASESIYSYLAWYQQKQGKSPQLLVYNTKTLPEGVPSRFSGSGSGTQFSLKINSLQPEDFGRYYCQHHYGTPPWTFGGGTNLEIK

SEQ ID NO: 16 - protein, 117 aa, Artificial Sequence
  Description of Artificial Sequence Synthetic polypeptide
EVQQVESGGDLVKPGGSLKLSCAASGFTFSSYGMSWVRQTPDKRLEWVATINSGGSNTYYPDSLKGRFTISRDNAKNTLYLQMRSLKSEDTAMYYCARHDGGAMDYWGQGTSVTVSS

SEQ ID NO: 17 - protein, 108 aa, Artificial Sequence
  Description of Artificial Sequence Synthetic polypeptide
DIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIK

SEQ ID NO: 18 - protein, 117 aa, Artificial Sequence
  Description of Artificial Sequence Synthetic polypeptide
EVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSS

SEQ ID NO: 19 - protein, 215 aa, Artificial Sequence
  Description of Artificial Sequence Synthetic polypeptide
DIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 447 aa, Artificial Sequence
  Description of Artificial Sequence Synthetic polypeptide
EVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 4 aa, Artificial Sequence
  Description of Artificial Sequence Synthetic peptide
LLQL

SEQ ID NO: 22 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 23 - protein, 5 aa, Artificial Sequence
  Description of Artificial Sequence Synthetic peptide
SYWMN

SEQ ID NO: 24 - protein, 17 aa, Artificial Sequence
  Description of Artificial Sequence Synthetic peptide
VIHPSDSETWLDQKFKD

SEQ ID NO: 25 - protein, 10 aa, Artificial Sequence
  Description of Artificial Sequence Synthetic peptide
EHYGTSPFAY

SEQ ID NO: 26 - protein, 16 aa, Artificial Sequence
  Description of Artificial Sequence Synthetic peptide
RASESVDNYGMSFMNW

SEQ ID NO: 27 - protein, 7 aa, Artificial Sequence
  Description of Artificial Sequence Synthetic peptide
AASNQGS

SEQ ID NO: 28 - protein, 9 aa, Artificial Sequence
  Description of Artificial Sequence Synthetic peptide
QQSKEVPYT

SEQ ID NO: 29 - protein, 218 aa, Artificial Sequence
  Description of Artificial Sequence Synthetic polypeptide
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 445 aa, Artificial Sequence
  Description of Artificial Sequence Synthetic polypeptide
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 31 - protein, 111 aa, Artificial Sequence
  Description of Artificial Sequence Synthetic polypeptide
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIK

SEQ ID NO: 32 - protein, 119 aa, Artificial Sequence
  Description of Artificial Sequence Synthetic polypeptide
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSS

SEQ ID NO: 33 - protein, 111 aa, Artificial Sequence [sequence omitted]

SEQ ID NO: 34 - protein, 120 aa, Artificial Sequence [sequence omitted]

SEQ ID NO: 35 - protein, 107 aa, Artificial Sequence [sequence omitted]

SEQ ID NO: 36 - protein, 113 aa, Artificial Sequence [sequence omitted]

SEQ ID NO: 37 - protein, 496 aa, Artificial Sequence
  Description of Artificial Sequence Synthetic polypeptide
DIQMTQSPSSLSASVGDRVTITCRASQDVSSVVAWYQQKPGKAPKLLIYSASYRYTGVPSRFSGSGSGTDFTLTISSLQPEDFATYYCQQHYSTPWTFGGGTKLEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGEVAACEKEVAALEKEVAALEKEVAALEKESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 38 - protein, 271 aa, Artificial Sequence
  Description of Artificial Sequence Synthetic polypeptide
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVQSGAEVKKPGASVKVSCKASGYTFTDYNMDWVRQAPGQGLEWMGDINPDNGVTIYNQKFEGRVTMTTDTSTSTAYMELRSLRSDDTAVYYCAREADYFYFDYWGQGTTLTVSSGGCGGGKVAACKEKVAALKEKVAALKEKVAALKE

SEQ ID NO: 39 - protein, 11 aa, Artificial Sequence
  Description of Artificial Sequence Synthetic peptide
RASESIYSYLA

SEQ ID NO: 40 - protein, 7 aa, Artificial Sequence
  Description of Artificial Sequence Synthetic peptide
NTKTLPE

SEQ ID NO: 41 - protein, 10 aa, Artificial Sequence
  Description of Artificial Sequence Synthetic peptide
QHHYGTPPWT

SEQ ID NO: 42 - protein, 5 aa, Artificial Sequence
  Description of Artificial Sequence Synthetic peptide
SYGMS

SEQ ID NO: 43 - protein, 17 aa, Artificial Sequence
  Description of Artificial Sequence Synthetic peptide
TINSGGSNTYYPDSLKG

SEQ ID NO: 44 - protein, 8 aa, Artificial Sequence
  Description of Artificial Sequence Synthetic peptide
HDGGAMDY

SEQ ID NO: 45 - protein, 107 aa, Artificial Sequence
  Description of Artificial Sequence Synthetic polypeptide
DIQMTQSPSSLSASVGDRVTITCRASQDVSSVVAWYQQKPGKAPKLLIYSASYRYTGVPSRFSGSGSGTDFTLTISSLQPEDFATYYCQQHYSTPWTFGGGTKLEIK

SEQ ID NO: 46 - protein, 118 aa, Artificial Sequence
  Description of Artificial Sequence Synthetic polypeptide
QVQLVQSGAEVKKPGASVKVSCKASGYTFTDYNMDWVRQAPGQGLEWMGDINPDNGVTIYNQKFEGRVTMTTDTSTSTAYMELRSLRSDDTAVYYCAREADYFYFDYWGQGTTLTVSS